(19) United States
(12) Patent Application Publication
(10) Pub. No.: US 2024/0335200 A1
(43) Pub. Date: Oct. 10, 2024

(54) METHODS, SYSTEMS, AND DEVICES FOR THE OCCLUSION OF THE LEFT ATRIAL APPENDAGE

(71) Applicant: APPLIED CARDIOVASCULAR SOLUTIONS, LLC, Austin, TX (US)

(72) Inventors: F. Javier OTERO, Austin, TX (US); Joseph J. BEAMAN, Austin, TX (US); Rodney Paul HORTON, Austin, TX (US); Andrea NATALE, Austin, TX (US); Joel W. BARLOW, Austin, TX (US)

(21) Appl. No.: 18/292,704

(22) PCT Filed: Jul. 27, 2022
(86) PCT No.: PCT/US2022/038576

§ 371 Date: Jan. 26, 2024

Related U.S. Application Data
(60) Provisional application No. 63/226,146, filed on Jul. 27, 2021.

(51) Int. Cl. A61B 17/12; A61B 17/00; A61L 24/00; A61L 24/04; A61M 25/00; A61M 25/10

(52) U.S. Cl. CPC A61B 17/12122; A61B 17/12177; A61B 17/12186; A61B 17/12195; A61L 24/0015; A61L 24/0031; A61L 24/043; A61B 2017/00579; A61B 2017/00632; A61B 2017/1205; A61L 2400/06; A61M 25/0097; A61M 25/1011; A61M 2025/1047

(57) ABSTRACT

Provided herein are methods, systems, and devices for occluding the LAA of a patient's heart. The methods, systems, and devices can be used to percutaneously occlude the LAA, decreasing the risk of thromboembolic events associated with AF.

### CROSS-REFERENCE TO RELATED APPLICATIONS

This application claims benefit of U.S. Provisional Application No. 63/226,146, filed Jul. 27, 2021, which is hereby incorporated herein by reference in its entirety.

### FIELD

The present disclosure is generally related to methods, systems, and devices for occluding the left atrial appendage (LAA) of a patient's heart.

### BACKGROUND

Embolic stroke is a leading cause of death and disability among adults. The most common cause of embolic stroke emanating from the heart is thrombus formation due to atrial fibrillation (AF). AF is an arrhythmia of the heart that results in a rapid and chaotic heartbeat, producing decreased cardiac output and leading to irregular and turbulent blood flow in the vascular system.

In the case of patients who exhibit AF and develop an atrial thrombus, clot formation typically occurs in the left atrial appendage (LAA) of the patient's heart. The LAA is a small cavity formed within the lateral wall of the left atrium between the mitral valve and the root of the left pulmonary vein. In normal hearts, the LAA contracts in conjunction with the rest of the left atrium during the cardiac cycle; however in the case of patients suffering from AF, the LAA often fails to contract with any vigor. As a consequence, blood can stagnate within the LAA, resulting in thrombus formation.

Elimination or containment of thrombus formed within the LAA offers the potential to significantly reduce the incidence of stroke in patients suffering from AF. Pharmacological therapies, for example the oral or systemic administration of anticoagulants such as warfarin, are often used to prevent thrombus formation. However, anticoagulant therapy is often undesirable or unsuccessful due to medication side effects (e.g., hemorrhage), interactions with foods and other drugs, and lack of patient compliance.

Invasive surgical or thorascopic techniques have been used to obliterate the LAA, however, many patients with AF are not suitable candidates for such surgical procedures due to a compromised condition or having previously undergone cardiac surgery. In addition, the perceived risks of surgical procedures often outweigh the potential benefits.

Recently, percutaneous occlusion implants for use in the LAA have been investigated as alternatives to anticoagulant therapy. However, these implants are relatively non-conforming. Due to the non-uniform shape of the LAA, existing implants cannot completely seal the opening of the LAA in all patients. As a consequence, approximately 15% of patients receiving these implants experience incomplete LAA closure, necessitating prolonged treatment with anticoagulants. The anatomy of the left atrium and LAA of some patients also precludes the use of such implants. In addition, the occlusion implants can also cause life-threatening perforations of the LAA during the placement procedure.

More effective methods of occluding cavities or passageways in a patient, in particular cavities or passageways in the cardiovascular system of a patient, such as the LAA, offer the potential to improve patient outcomes while eliminating the undesirable consequences of existing therapies.

### SUMMARY

Provided are methods, systems, and devices for occluding the LAA of a patient's heart. These methods, systems, and devices can be used to decrease the rate of thromboembolic events associated with AF by occluding the LAA.

Methods for occluding the LAA of a patient can involve injecting a fluid biomaterial into the LAA of the patient. The fluid biomaterial exhibits a cure time following injection. During the cure time, fluid biomaterial remains flowable, allowing it to comply with the irregular shape of the interior of the LAA. Methods can further involve positioning an occlusion device within the ostium of the LAA. The occlusion device can comprise an occluder portion and an anchor portion coupled to the occluder portion. When the occlusion device is positioned within the ostium of the LAA, the anchor portion extends into the internal volume of the LAA.

After the cure time has elapsed, the fluid biomaterial solidifies (e.g., as a result of crosslinking, a phase change induced as a consequence of injection of the composition into a physiological environment, or any combination thereof) to form a biocompatible polymeric matrix that fills and occupies the internal volume of the LAA. Because of the compliant nature of the fluid biomaterial, the resulting biocompatible polymeric matrix can be interpenetrated by both the anchor portion of the occlusion device and trabeculae present in the LAA. In this way, the biocompatible polymeric matrix ensures that the occlusion device is retained within the ostium of the LAA. Further, the biocompatible matrix can function as an occlusive body, filling and occupying the internal volume of the LAA without adversely impacting cardiac function. In addition, the occlusion device can isolate the biocompatible polymeric matrix from blood present in the left atrium, provide a scaffold for endothelialization, of a combination thereof.

In some embodiments, the occlusion device can be positioned within the ostium of the LAA prior to injection of the fluid biomaterial into the LAA. For example, methods for occluding the LAA of a patient can involve positioning an occlusion device within the ostium of the LAA, wherein the occlusion device comprises an occluder portion comprising a proximal end and a distal end, the proximal end coupled to a hub having an injection lumen passing axially therethrough; and an anchor portion operably coupled to the occluder portion. The occlusion device can be positioned within the ostium of the LAA such that the anchor portion extends into the internal volume of the LAA. A fluid biomaterial can then be injected into the LAA of the patient through the injection lumen of the occlusion device. The fluid biomaterial exhibits a cure time following injection during which it remains flowable but after which it solidifies to form a biocompatible polymeric matrix that fills and occupies the internal volume of the LAA. The occlusion device can be retained within the ostium of the LAA until the cure time has elapsed and the fluid biomaterial has solidified to form the biocompatible polymeric matrix.

In other embodiments, the occlusion device can be positioned within the ostium of the LAA after injection of the fluid biomaterial into the LAA. For example, methods for occluding the LAA of a patient can involve injecting a fluid biomaterial into the LAA of the patient. The fluid biomaterial exhibits a cure time following injection. During the cure time, fluid biomaterial remains flowable, allowing it to comply with the irregular shape of the interior of the LAA. During this cure time, an occlusion device can be positioned within the ostium of the LAA. The occlusion device can comprise an occluder portion and an anchor portion operably coupled to the occluder portion. When the occlusion device is positioned within the ostium of the LAA, the anchor portion extends into the internal volume of the LAA. The occlusion device can be retained within the ostium of the LAA until the cure time of the fluid biomaterial has elapsed. After the cure time has elapsed, the fluid biomaterial solidifies (e.g., as a result of crosslinking, a phase change induced as a consequence of injection of the composition into a physiological environment, or any combination thereof) to form a biocompatible polymeric matrix that fills and occupies the internal volume of the LAA. Because of the compliant nature of the fluid biomaterial, the resulting biocompatible polymeric matrix can be interpenetrated by both the anchor portion of the occlusion device and trabeculae present in the LAA.

In other embodiments, the occlusion device can comprise an occluder portion comprising a proximal end and a distal end, the proximal end coupled to a hub having an injection lumen passing axially therethrough. Methods of occluding the LAA of a patient can comprise positioning the occlusion device within the ostium of the LAA, injecting a fluid biomaterial into the LAA of the patient through the injection lumen, wherein the fluid biomaterial exhibits a cure time following injection during which it remains flowable but after which it solidifies to form a biocompatible polymeric matrix that fills and occupies the internal volume of the LAA; and advancing an anchoring portion through the injection lumen and coupling the anchoring portion to the occlusion device, wherein when the anchoring portion is coupled to the occlusion device, the anchor portion extends into the internal volume of the LAA. In some embodiments, the anchor portion can be advanced before the cure time has elapsed, and the method can further comprise retaining the occlusion device within the ostium of the LAA until the cure time has elapsed and the fluid biomaterial has solidified to form the biocompatible polymeric matrix. In other embodiments, the anchor portion can be structured to be advanced through a solidified biocompatible polymeric matrix (e.g., the anchor portion can comprise one or more structures similar to barbed needles). In these embodiments, the anchor portion can be advanced before or after the fluid biomaterial has solidified.

Also provided are methods of occluding the left atrial appendage (LAA) of a patient that comprise injecting a fluid biomaterial comprising a silencing agent dissolved or dispersed therein into the LAA of the patient, wherein the fluid biomaterial solidifies in situ in the LAA to form a biocompatible polymeric matrix that fills and occupies the LAA. The silencing agent can comprise any suitable agent (small molecule or biologic) that can be locally released from the fluid biomaterial (and/or the biocompatible polymer matrix) and eliminates contractility of cardiac tissue in the walls of the LAA (e.g., by interrupting electrical signals, inducing apoptosis, etc.). In some embodiments, the biocompatible polymer matrix can provide for localized, controlled release of the silencing agent to cardiac tissue in the walls of the LAA over a period of at least 2 weeks. In certain embodiments, the apoptotic agent can comprise aclarubicin, an apoptosis gene modulator, an apoptosis regulator, an arginine deaminase, clotrimazole, curacin A, etoposide, gemcitabine, a ras inhibitor, a ras-GAP inhibitor, a topoisomerase inhibitor such as topotecan or camptothecin, a taxane such as docetaxel or paclitaxel, an anthracycline, a cyclophosphamide, a vinca alkaloid, a plantinum-based chemotherapeutic agent such as cisplatin or carboplatin, 5-fluoro-uracil, gemcitabine, capecitabin, navelbine, zoledronate, venetoclax, ABT-737, or any combination thereof.

### BRIEF DESCRIPTION OF THE FIGURES

FIG. 1 is an anterior illustration of a heart, including proximal portions of the great vessels.

FIG. 2A is a perspective view of an example occlusion device and a distal portion of an example delivery system. FIG. 2B is a partial cross-sectional view of the occlusion device, taken along section line 1A of FIG. 2A. FIG. 2C is an enlarged section view of an occluder portion, taken from detail 2B of FIG. 2B.

FIG. 3A is a side view of an example occlusion device and an example delivery system FIG. 3B is a side view of an example occlusion device employed with the delivery system of FIG. 3B, depicting the occlusion device being implanted in a left atrial appendage.

FIG. 4 is a perspective view of the occlusion device of FIG. 3A, depicting the medical device in a fully expanded position.

FIG. 5 is a side view of the occlusion device shown in FIG. 4.

FIG. 6A is a side view of an example occlusion device and an example delivery system FIG. 6B is a side view of an example occlusion device employed with the delivery system of FIG. 6B, depicting the occlusion device being implanted in a left atrial appendage.

FIG. 7A-7C illustrate another example occlusion device.

FIG. 8A is a schematic illustration of an exemplary first catheter body and first balloon, as described herein. As shown, the first catheter body can have a proximal end portion, a distal end portion having a tip, and a wall that circumferentially encloses a primary opening. FIG. 8B is a cross-sectional side view of the first catheter body taken along line 1B-1B of FIG. 8A. As shown, the first catheter body can have at least one inflation channel within the wall of the first catheter body. The primary opening of the first catheter body can extend an entire length of the first catheter body. FIG. 8C is a close-up view of a portion of the first catheter body of FIG. 8A showing at least one outlet opening defined therein to provide fluid communication between the at least one inflation channel and the interior space of the first balloon, as described herein.

FIG. 8D is a cross-sectional side view of the portion of the first catheter body of FIG. 8C taken along line 1D-1D, as described herein.

FIG. 9A is a schematic illustration of an exemplary second catheter body and second balloon, as described herein. As shown, the second catheter body can have a proximal end portion, a distal end portion having a tip, and a wall that circumferentially encloses a primary opening. FIG. 9B is a cross-sectional side view of the second catheter body taken along line 2B-2B of FIG. 9A, as described herein. As shown, the second catheter body can have at least one inflation channel within the wall of the second catheter body, and the primary opening of the second catheter body can extend an entire length of the second catheter body. FIG. 9C is a close-up view of a portion of the second catheter body of FIG. 9A showing at least one outlet opening defined therein to provide fluid communication between the at least one inflation channel and the interior space of the second balloon, as described herein.

FIG. 10A is a schematic illustration of an exemplary third catheter body, as described herein. As shown, the third catheter body can include a proximal end portion, a distal end portion, and a wall structure that defines at least one injection channel extending from the proximal end portion toward the distal end portion. FIG. 10B is a cross-sectional side view of the third catheter body taken along line 3B-3B of FIG. 10A, which shows the least one injection channel. FIG. 10C is a close-up view of the distal end portion of the third catheter body of FIG. 10A, as described herein. FIG. 10D is a cross-sectional side view of the distal end portion taken along line 3D-3D of FIG. 10C. As shown and described herein, the distal end portion can at least one outlet opening positioned in fluid communication with the at least one injection channel.

FIG. 11 is a perspective view of an exemplary catheter assembly, as described herein.

FIGS. 12A-12C illustrate an example method of occluding the LAA.

FIGS. 13A-13D illustrate an example method of occluding the LAA.

FIGS. 14A-14E illustrate an example method of occluding the LAA.

### DETAILED DESCRIPTION

Before the present methods, compositions, systems, articles, devices, and/or methods are disclosed and described, it is to be understood that the aspects described below are not limited to specific compounds, specific devices, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Examples of longer chain alkyl groups include, but are not limited to, a palmitate group.

A “lower alkyl” group is an alkyl group containing from one to six carbon atoms.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulfur, or phosphorus.

The term “aryl group” as used herein is any carbon-based aromatic group including, but not limited to, benzene, naphthalene, etc. The term “aryl group” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. In one aspect, the heteroaryl group is imidazole. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “nucleophilic group” includes any groups capable of reacting with an activated ester. Examples include amino groups, thiols groups, hydroxyl groups, and their corresponding anions.

The term “carboxyl group” includes a carboxylic acid and the corresponding salt thereof.

The term “amino group” as used herein is represented as the formula —NHRR′, where R and R′ can be any organic group including alkyl, aryl, carbonyl, heterocycloalkyl, and the like, where R and R′ can be separate groups or be part of a ring. For example, pyridine is a heteroaryl group where R and R′ are part of the aromatic ring.

The term “treat” as used herein is defined as maintaining or reducing the symptoms of a pre-existing condition. The term “prevent” as used herein is defined as eliminating or reducing the likelihood of the occurrence of one or more symptoms of a disease or disorder. The term “reduce” as used herein is the ability of the in situ solidifying complex coacervate described herein to completely eliminate the activity or reduce the activity when compared to the same activity in the absence of the complex coacervate.

“Subject” refers to mammals including, but not limited to, humans, non-human primates, sheep, dogs, rodents (e.g., mouse, rat, etc.), guinea pigs, cats, rabbits, cows, and non-mammals including chickens, amphibians, and reptiles.

“Physiological conditions” refers to condition such as pH, temperature, etc. within the subject. For example, the physiological pH and temperature of a human is 7.2 and 37° C., respectively.

The term “interpenetrating polymer network” (IPN) refers to a polymer network that comprises an intermixture of two or more polymers that are physically entangled but not chemically linked (i.e., two or more networks which are at least partially interlaced on a polymer scale but not covalently bonded to each other). Generally, the polymers in the IPN cannot be separated unless chemical bonds are broken. The two or more networks can be envisioned to be entangled in such a way that they are concatenated and cannot be pulled apart, but not bonded to each other by any chemical bond. The term IPN includes networks formed by simultaneous synthesis processes, networks formed by sequential synthesis processes, and “semi-IPN” systems that include a linear, non-crosslinked polymer that is physically entangled within a crosslinked polymer network. The term IPN can also include interconnected polymer networks that include a limited amount of inter-network chemical links.

The term “inverse thermosensitive” refers to the property of a polymer wherein gelation and/or solidification (precipitation) takes place upon an increase in temperature, rather than a decrease in temperature. The term “transition temperature” refers to the temperature or temperature range at which gelation and/or solidification (precipitation) of an inverse thermosensitive polymer occurs.

The term “inverse thermosensitive polymer” as used herein refers to a polymer that is a liquid and/or is soluble in water at first temperature below body temperature (e.g., 4° C. or 23° C.), but which forms a solid or gel or at least partially phase-separates out of water, at physiological temperature (e.g., 37° C.). Examples of inverse thermosensitive polymers include poloxamer 407 (commercially available under the tradename PLURONIC® F127), poloxamer 188 (commercially available under the tradename PLURONIC® F68), poly(N-isopropylacrylamide), poly(methyl vinyl ether), poly(N-vinylcaprolactam), and certain poly(organophosphazenes). Such materials are described in Bull. Korean Chem. Soc. 2002, 23, 549-554, which is hereby incorporated by reference in its entirety.

The term “biocompatible”, as used herein, refers to having the property of being biologically compatible by not producing a toxic, injurious, or immunological response in living tissue.

The term “poloxamer” denotes a symmetrical block copolymer, consisting of a core of PPG polyoxyethylated to both its terminal hydroxyl groups, i.e. conforming to the interchangeable generic formula (PEG)_{X}-(PPG)_{Y}-(PEG)_{X} and (PEO)_{X}-(PPO)_{Y}-(PEO)_{X}. Each poloxamer name ends with an arbitrary code number, which is related to the average numerical values of the respective monomer units denoted by X and Y.

The term “poloxamine” denotes a polyalkoxylated symmetrical block copolymer of ethylene diamine conforming to the general type [(PEG)_{X}-(PPG)_{Y}]₂—NCH₂CH₂N-[(PPG)_{Y}-(PEG)_{X}]₂. Each Poloxamine name is followed by an arbitrary code number, which is related to the average numerical values of the respective monomer units denoted by X and Y.

The phrase “polydispersity index” refers to the ratio of the “weight average molecular weight” to the “number average molecular weight” for a particular polymer; it reflects the distribution of individual molecular weights in a polymer sample.

The phrase “weight average molecular weight” refers to a particular measure of the molecular weight of a polymer. The weight average molecular weight is calculated as follows: determine the molecular weight of a number of polymer molecules; add the squares of these weights; and then divide by the total weight of the molecules.

The phrase “number average molecular weight” refers to a particular measure of the molecular weight of a polymer. The number average molecular weight is the common average of the molecular weights of the individual polymer molecules. It is determined by measuring the molecular weight of n polymer molecules, summing the weights, and dividing by n.

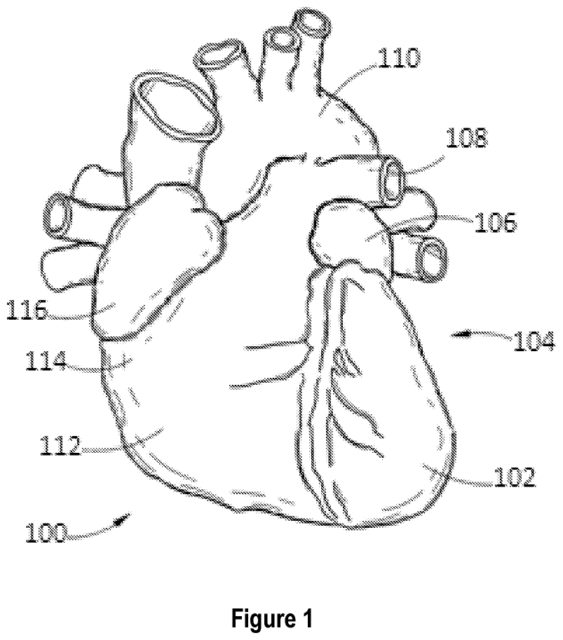

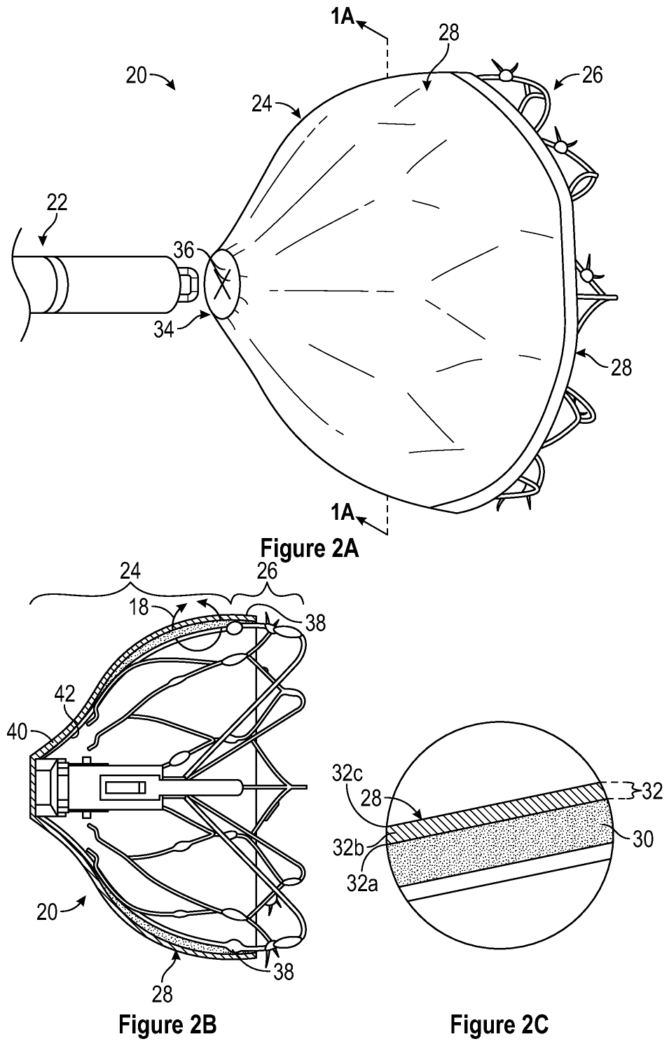

To facilitate understanding of the physiology associated with the methods, compositions, and devices described herein, FIG. 1 illustrates the anatomy of the human heart (100). Referring to FIG. 1, the heart 100 is illustrated to show certain portions including the left ventricle (102), the left atrium (104), the LAA (106), the pulmonary artery (108), the aorta (110), the right ventricle (112), the right atrium (114), and the right atrial appendage (116). The left atrium is located above the left ventricle, and is separated from the left ventricle by the mitral valve (not illustrated). As shown in FIG. 2A, the LAA (106) can have an irregular finger-like or windsock shape with an opening (also referred to as an ostium, 120) approximately 1.5 cm in diameter. The internal volume of a normal LAA is approximately 9.3 f 3.5 mL. The LAA is normally in fluid communication with the left atrium such that blood flows in and out of the LAA as the heart beats.

Provided are methods, systems, and devices for occluding the LAA of a patient's heart. Methods for occluding the LAA of a patient can involve injecting a fluid biomaterial into the LAA of the patient. The fluid biomaterial exhibits a cure time following injection. During the cure time, fluid biomaterial remains flowable, allowing it to comply with the irregular shape of the interior of the LAA. During this cure time, an occlusion device can be positioned within the ostium of the LAA. The occlusion device can comprise an occluder portion and an anchor portion operably coupled to the occluder portion. When the occlusion device is positioned within the ostium of the LAA, the anchor portion extends into the internal volume of the LAA. The occlusion device can be retained within the ostium of the LAA until the cure time of the fluid biomaterial has elapsed.

After the cure time has elapsed, the fluid biomaterial solidifies (e.g., as a result of crosslinking, a phase change induced as a consequence of injection of the composition into a physiological environment, or any combination thereof) to form a biocompatible polymeric matrix that fills and occupies the internal volume of the LAA. Because of the compliant nature of the fluid biomaterial, the resulting biocompatible polymeric matrix can be interpenetrated by both the anchor portion of the occlusion device and trabeculae present in the LAA. In this way, the biocompatible polymeric matrix ensures that the occlusion device is retained within the ostium of the LAA. Further, the biocompatible matrix can function as an occlusive body, filling and occupying the internal volume of the LAA without adversely impacting cardiac function.

### Fluid Biomaterials

The methods described herein involve injection of a fluid biomaterial into the LAA of a subject. Following injection, the fluid biomaterials solidify to form a biocompatible polymeric matrix in situ within the LAA, filling and occupying the internal volume of the LAA.

A variety of suitable fluid biomaterials can be used, including stimuli-responsive materials (e.g., ionically responsive complex coacervates which undergo a phase transition upon injection into the LAA) and crosslinkable biomaterials (e.g., compositions that include precursor molecules that are substantially uncrosslinked prior to and at the time of injection but which crosslink by forming covalent and/or non-covalent linkages with each other at the site upon injection into the LAA). Examples of suitable materials include those described in WO 2014/160083, which is hereby incorporated by reference in its entirety. In some embodiments, the fluid biomaterial can comprise an IPN or composition which forms an IPN in situ in the LAA following injection.

The fluid biomaterial as well as the resultant biocompatible polymeric matrix can be selected to possess suitable materials properties (e.g., viscosity, cohesive strength, adhesive strength, elasticity, degradation rate, swelling behavior, cure time, etc.) for use in occlusion of the LAA.

For example, the biocompatible polymeric matrix can exhibit an equilibrium swelling ratio suitable for occlusion of the LAA. Swelling refers to the uptake of water or biological fluids by the biocompatible polymeric matrix. The swelling of the biocompatible polymeric matrix can be quantified using the equilibrium swelling ratio, defined as the mass of the biocompatible polymeric matrix at equilibrium swelling (i.e., the materials maximum swollen weight) divided by the mass of the biocompatible polymeric matrix prior to swelling (e.g., immediately following curing). In many cases, equilibrium swelling is reached within a relatively short period of time (e.g., within about 24-48 hours).

In some embodiments, the biocompatible polymeric matrix exhibits an equilibrium swelling ratio of less than about 8 (e.g., less than about 7.5, less than about 7.0, less than about 6.5, less than about 6.0, less than about 5.5, less than about 5.0, less than about 4.5, less than about 4.0, less than about 3.5, less than about 3.0, less than about 2.5, less than about 2.0, less than about 1.5, less than about 1.0, or less than about 0.5). In some embodiments, the biocompatible polymeric matrix exhibits an equilibrium swelling ratio of greater than 0 (e.g., greater than about 0.5, greater than about 1.0, greater than about 1.5, greater than about 2.0, greater than about 2.5, greater than about 3.0, greater than about 3.5, greater than about 4.0, greater than about 4.5, greater than about 5.0, greater than about 5.5, greater than about 6.0, greater than about 6.5, greater than about 7.0, or greater than about 7.5).

The biocompatible polymeric matrix can exhibit an equilibrium swelling ratio ranging from any of the minimum values described above to any of the maximum values described above. For example, the biocompatible polymeric matrix can exhibit an equilibrium swelling ratio of from greater than 0 to about 8.0 (e.g., from about 2.0 to about 8.0, of from about 2.5 to about 6.0).

In some embodiments, the biocompatible polymeric matrix exhibits a volumetric swelling ratio of less than about 15 (e.g., less than about 12, less than about 10, less than about 9, less than about 8, less than about 7, less than about 6, less than about 5, less than about 4, less than about 3, less than about 2, or less than about 1). In some embodiments, the biocompatible polymeric matrix exhibits an equilibrium swelling ratio of greater than 0 (e.g., greater than about 1, greater than about 2, greater than about 3, greater than about 4, greater than about 5, greater than about 6, greater than about 7, greater than about 8, greater than about 9, greater than about 10, or greater than about 12).

The biocompatible polymeric matrix can exhibit a volumetric swelling ratio ranging from any of the minimum values described above to any of the maximum values described above. For example, the biocompatible polymeric matrix can exhibit an equilibrium swelling ratio of from greater than 0 to about 15 (e.g., from about 2 to about 15).

The biocompatible polymeric matrix can have mechanical properties that are compatible with cardiac function, such that the presence of the biocompatible polymeric matrix within the LAA does not substantially impede or inhibit cardiac function. For example, the biocompatible polymeric matrix can be formed to be at least partially compliant with the constrictive action of the heart muscle throughout the cardiac cycle. Suitable biocompatible polymeric matrices can have elastic moduli ranging from about 0.01 to about 100 kPa.

In some cases, biocompatible polymeric matrix is formed to have an elastic modulus similar to that of cardiac tissue. In some embodiments, the biocompatible polymeric matrix has an elastic modulus greater than about 5 kPa (e.g., greater than about 6 kPa, greater than about 7 kPa, greater than about 8 kPa, greater than about 9 kPa, greater than about 10 kPa, greater than about 11 kPa, greater than about 12 kPa, greater than about 13 kPa, greater than about 14 kPa, greater than about 15 kPa, greater than about 16 kPa, greater than about 17 kPa, greater than about 18 kPa, or greater than about 19 kPa. In some embodiments, the biocompatible polymeric matrix has an elastic modulus of less than about 20 kPa (e.g., less than about 19 kPa, less than about 18 kPa, less than about 17 kPa, less than about 16 kPa, less than about 15 kPa, less than about 14 kPa, less than about 13 kPa, less than about 12 kPa, less than about 11 kPa, less than about 10 kPa, less than about 9 kPa, less than about 8 kPa, less than about 7 kPa, or less than about 6 kPa).

The biocompatible polymeric matrix can have an elastic modulus ranging from any of the minimum values described above to any of the maximum values described above. For example, the biocompatible polymeric matrix can have an elastic modulus of from about 5 kPa to about 20 kPa (e.g., from about 9 kPa to about 17 kPa, from about 10 kPa to about 15 kPa, or from about 8 kPa to about 12 kPa).

In other embodiments, the biocompatible polymeric matric can have an elastic modulus of greater than 20 kPa (e.g., from greater than 20 kPa to about 13000 kPa, from greater than 20 kPa to 10000 kPa).

The biocompatible polymeric matrix can have a cohesive strength suitable for occlusion of the LAA. Cohesive strength (also referred to as burst strength) refers to the ability of the biocompatible polymeric matrix to remain intact (i.e., not rupture, tear or crack) when subjected to physical stresses or environmental conditions. The cohesive strength of the biocompatible polymeric matrix can be measured using methods known in the art, for example, using the standard methods described in ASTM F-2392-04 (standard test for the burst strength of surgical sealants). In some embodiments, the biocompatible polymeric matrix has a cohesive strength effective such that the biocompatible polymeric matrix remains intact (e.g., does not fragment or break apart into smaller pieces which exit the LAA) for a period of time effective to permit the LAA to be sealed via endothelialization prior to fragmentation of the biocompatible polymeric matrix.

The biocompatible polymeric matrix can also have a viscosity which minimizes migration of biocompatible polymeric matrix out of the LAA following injection. In some embodiments, the biocompatible polymeric matrix has a viscosity of at least 50,000 cP (e.g., at least 60,000 cP, at least 70,000 cP, at least 75,000 cP, at least 80,000 cP, at least 90,000 cP, at least 100,000 cP, or more) at body temperature (e.g., at 37° C.).

The biocompatible matrix can also be selected such that is it retained at the site of occlusion (e.g., inside the LAA) by a combination of adhesion to the tissues at the site of occlusion and mechanical interaction with the anatomy at the site of occlusion. For example, the biocompatible polymeric matrix can have an adhesive strength suitable for occlusion of the LAA. Adhesive strength refers to the ability of the biocompatible polymeric matrix to remain attached to the tissues at the site of administration (e.g., the interior of the LAA) when subjected to physical stresses or environmental conditions. In some embodiments, the biocompatible polymeric matrix has an adhesive strength effective such that the biocompatible polymeric matrix remains within the LAA (e.g., does not exit the LAA) for a period of time effective to permit the LAA to be sealed via endothelialization prior to fragmentation of the biocompatible polymeric matrix. Mechanical forces, governed by a combination of properties which can include the swelling of the biocompatible polymeric matrix, the local anatomy at the site of injection (e.g., the particular 3-dimensional shape of the LAA and/or the surface texture of the LAA interior), and the friction of the biocompatible polymeric matrix against tissue at the site of injection, can also contribute to retention of the biocompatible matrix at the site of injection.

The biocompatible polymeric matrix can be formed from materials which support (i.e., do not inhibit) endothelialization. Endothelialization refers to the growth and/or proliferation of endothelial cells on a surface, such as the blood-contacting surface, of the biocompatible polymeric matrix or a surface of the occlusion device. These materials can be biodegradable or non-biodegradable. A biodegradable material is one which decomposes under normal in vivo physiological conditions into components which can be metabolized or excreted.

In certain cases, the biocompatible polymeric matrix can be non-biodegradable. In some embodiments, the biocompatible polymeric matrix has a degradation rate such that about 25% or less by weight of the biocompatible polymeric matrix degrades within 90 days of curing, as measured using the standard method described in Example 1 (e.g., about 20% or less by weight, about 15% or less by weight, about 10% or less by weight, about 5% or less by weight, about 2.5% or less by weight, or less).

Suitable biocompatible polymeric matrices can be formed from a variety of natural and/or synthetic materials. In certain embodiments, the biocompatible polymeric matrix is a hydrogel. Hydrogels are water-swellable materials formed from oligomeric or polymeric molecules which are crosslinked to form a three dimensional network. Hydrogels can be designed to form in situ (for example, from injectable precursors which crosslink in vivo). As gels, these materials can exhibit properties characteristic of both liquids (e.g., their shape can be resilient and deformable) and solids (e.g., their shape can be discrete enough to maintain three dimensions on a two dimensional surface).

The fluid biomaterial can be designed to rapidly cure in situ upon injection. In some embodiments, the fluid biomaterial has a cure time, as measured using the standard method described in Example 1, of less than about 20 minutes (e.g., less than about 15 minutes, less than about 10 minutes, less than about 5 minutes, less than about 3 minutes, or less than about 1 minute).

The fluid biomaterial injected into the LAA can have a low viscosity relative to the biocompatible polymeric matrix. This can allow the fluid biomaterial to be readily injected, for example, via a hand-powered delivery device such as a syringe. This can provide a physician with a large degree of control over the flow rate of the fluid biomaterial during injection, and allow the flow to be altered or stopped, as required, during the course of injection. The relatively low viscosity of the fluid biomaterial relative to the biocompatible polymeric matrix also can allow the fluid biomaterial to conform to the shape of the LAA prior to curing and intimately interact with both trabeculae in the LAA as well as the anchor portion of the occlusion device.

For example, in some embodiments, the fluid biomaterial injected into the LAA has a viscosity of about 2,000 cP or less (e.g., about 1,500 cP or less, about 1,250 cP or less, about 1,000 cP or less, about 900 cP or less, about 800 cP or less, about 750 cP or less, about 700 cP or less, about 600 cP or less, about 500 cP or less, about 400 cP or less, about 300 cP or less, about 250 cP or less, about 200 cP or less, about 150 cP or less, about 100 cP or less, or. about 50 cP or less) at room temperature. In some embodiments, the viscosity of the fluid biomaterial can be greater than the viscosity of human blood. In certain cases, the fluid biomaterial is injected into the LAA has a viscosity of at least 1 cP (e.g., at least 2 cP, at least 2.5 cP, at least 5 cP, or at least 10 cP) at room temperature. The fluid biomaterial can have a viscosity ranging from any of the minimum values described above to any of the maximum values described above.

Suitable fluid biomaterials include stimuli-responsive materials (e.g., inverse thermosensitive polymers and/or ionically responsive complex coacervates) and crosslinkable biomaterials. Examples of suitable fluid biomaterials are discussed in more detail below.

### Ionically Responsive Complex Coacervates

In some embodiments, the fluid biomaterial can comprise an ionically responsive complex coacervate. Examples of such compositions are described, for example, in International Patent Publication No. WO 2016/011028 to University of Utah Research Foundation, which is hereby incorporated by reference in its entirety.

Polyelectrolytes with opposite net charges in aqueous solution can associate into several higher order morphologies depending on the solution conditions and charge ratios. They can form stable colloidal suspensions of polyelectrolyte complexes with net surface charges. Repulsion between like surface charges stabilize the suspension from further association. When the polyelectrolyte charge ratios are balanced, or near balance, the initial complexes can further coalesce and settle out into a dense fluid phase in which the opposite macroion charges are approximately equal. This process is referred to as complex coacervation, and the dense fluid morphology as a complex coacervate. More descriptively, the process is an associative macrophase separation of an aqueous solution of two oppositely charged polyelectrolytes into two liquid phases, a dense concentrated polyelectrolyte phase in equilibrium with a polyelectrolyte depleted phase. The aqueous coacervate phase can be dispersed into the aqueous depleted phase but quickly settles back out, like oil droplets in water. The spontaneous demixing of paired polyelectrolytes into complex coacervates occurs when attractive forces between polyelectrolyte pairs are stronger than repulsive forces. In thermodynamic terms, the net negative change in free energy that drives complex coacervation derives primarily from the gain in entropy of the small counterions released when macroions associate, which overcomes the loss of configurational entropy of the fully solvated polyelectrolytes.

Different morphologies can be produced from polyelectrolytes with opposite net charges. By varying parameters such as charge ratio of the polyelectrolytes, temperature, salt concentration, and pH can result in the formation of a gel, a complex coacervate, or a clear homogeneous solution, i.e., no phase separation, in such systems. By mixing polyelectrolytes in a region of the phase diagram in which fluid complex coacervates form, the in situ solidifying complex coacervates can be prepared in a fluid form. If the fluid form is introduced into an environment corresponding to a gel region of the phase diagram, the fluid form will harden into a solid gel as the in situ solidifying complex coacervate equilibrates to the new solution conditions. The term “gel” is defined herein as non-fluid colloidal network or polymer network that is expanded throughout its whole volume by a fluid. IUPAC. Compendium of Chemical Terminology, 2nd ed. (the “Gold Book”). Compiled by A. D. McNaught and A. Wilkinson. Blackwell Scientific Publications, Oxford (1997). Conversely, the fluid complex coacervates described herein are liquids. Thus, the fluid complex coacervates described herein have a completely different morphology compared to corresponding gels produced in situ despite the fact that the polycation and polyanion in the fluid complex coacervate and the gel are identical.

Suitable ionically responsive complex coacervates can comprise at least one polycation, at least one polyanion, and a monovalent salt. The concentration of the monovalent salt in the ionically responsive complex coacervate can be greater than the concentration of the monovalent salt in the LAA (i.e., at the site of injection). The components used to produce the in situ solidifying complex coacervates described herein as well as their applications thereof are provided below.

### Polycations

The polycation is generally composed of a polymer backbone with a plurality of cationic groups at a particular pH. The cationic groups can be pendant to the polymer backbone and/or incorporated within the polymer backbone. The polycation is any biocompatible polymer possessing cationic groups or groups that can be readily converted to cationic groups by adjusting the pH. In one aspect, the polycation is a polyamine compound. The amino groups of the polyamine can be branched or part of the polymer backbone. The amino group can be a primary, secondary, or tertiary amino group that can be protonated to produce a cationic ammonium group at a selected pH. In general, the polyamine is a polymer with a large excess of positive charges relative to negative charges at the relevant pH, as reflected in its isoelectric point (pI), which is the pH at which the polymer has a net neutral charge. The number of amino groups present on the polycation ultimately determines the charge density of the polycation at a particular pH. For example, the polycation can have from 10 to 90 mole %, 10 to 80 mole %, 10 to 70 mole %, 10 to 60 mole %, 10 to 50 mole %, 10 to 40 mole %, 10 to 30 mole %, or 10 to 20 mole % amino groups. In one aspect, the polyamine has excess positive charges at a pH of about 7, with a pI significantly greater than 7. Optionally, additional amino groups can be incorporated into the polymer in order to increase the pI value.

In one aspect, the amino group can be derived from a residue of lysine, histidine, or arginine attached to the polycation. For example, arginine has a guanidinyl group, where the guanidinyl group is a suitable amino group useful herein. Any anionic counterions can be used in association with the cationic polymers. The counterions should be physically and chemically compatible with the essential components of the composition and do not otherwise unduly impair product performance, stability or aesthetics. Non-limiting examples of such counterions include halides (e.g., chloride, fluoride, bromide, iodide), sulfate, methylsulfate, acetate and other monovalent carboxylic acids.

In one aspect, the polycation can be a positively-charged protein produced from a natural organism. For example, a recombinant P. californica protein can be used as the polycation. In one aspect, Pc1, Pc2, Pc4-Pc18 (SEQ ID NOS 1-17) can be used as the polycation. The type and number of amino acids present in the protein can vary in order to achieve the desired solution properties. For example, Pc1 is enriched with lysine (13.5 mole %) while Pc4 and Pc5 are enriched with histidine (12.6 and 11.3 mole %, respectively).

In another aspect, the polycation is a recombinant protein produced by artificial expression of a gene or a modified gene or a composite gene containing parts from several genes in a heterologous host such as, for example, bacteria, yeast, cows, goats, tobacco, and the like.

In another aspect, the polycation can be a biodegradable polyamine. The biodegradable polyamine can be a synthetic polymer or naturally-occurring polymer. The mechanism by which the polyamine can degrade will vary depending upon the polyamine that is used. In the case of natural polymers, they are biodegradable because there are enzymes that can hydrolyze the polymer chain. For example, proteases can hydrolyze natural proteins like gelatin. In the case of synthetic biodegradable polyamines, they also possess chemically labile bonds. For example, β-aminoesters have hydrolyzable ester groups. In addition to the nature of the polyamine, other considerations such as the molecular weight of the polyamine and crosslink density of the adhesive can be varied in order to modify the rate of biodegradability.

In one aspect, the biodegradable polyamine includes a polysaccharide, a protein, or a synthetic polyamine. Polysaccharides bearing one or more amino groups can be used herein. In one aspect, the polysaccharide is a natural polysaccharide such as chitosan or chemically modified chitosan. Similarly, the protein can be a synthetic or naturally-occurring compound. In another aspect, the biodegradable polyamine is a synthetic polyamine such as poly(β-aminoesters), polyester amines, poly(disulfide amines), mixed poly(ester and amide amines), and peptide crosslinked polyamines.

In the case when the polycation is a synthetic polymer, a variety of different polymers can be used; however, in certain applications such as, for example, biomedical applications, it is desirable that the polymer be biocompatible and non-toxic to cells and tissue. In one aspect, the biodegradable polyamine can be an amine-modified natural polymer. For example, the amine-modified natural polymer can be gelatin modified with one or more alkylamino groups, heteroaryl groups, or an aromatic group substituted with one or more amino groups. Examples of alkylamino groups are depicted in Formulae IV-VI below.

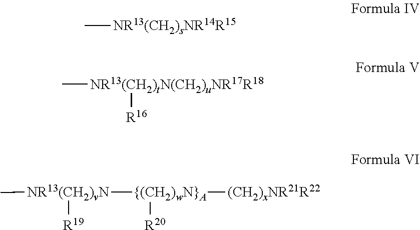

wherein R¹³-R²² are, independently, hydrogen, an alkyl group, or a nitrogen containing substituent;
s, t, u, v, w, and x are an integer from 1 to 10; and
A is an integer from 1 to 50,
where the alkylamino group is covalently attached to the natural polymer. In one aspect, if the natural polymer has a carboxyl group (e.g., acid or ester), the carboxyl group can be reacted with an alkyldiamino compound to produce an amide bond and incorporate the alkylamino group into the polymer. Thus, referring to formulae IV-VI, the amino group NR³ is covalently attached to the carbonyl group of the natural polymer.

As shown in Formulae IV-VI, the number of amino groups can vary. In one aspect, the alkylamino group is —NHCH₂NH₂, —NHCH₂CH₂NH₂, —NHCH₂CH₂CH₂NH₂, —NHCH₂CH₂CH₂CH₂NH₂, —NHCH₂CH₂CH₂CH₂CH₂NH₂, —NHCH₂NHCH₂CH₂CH₂NH₂, —NHCH₂CH₂NHCH₂CH₂CH₂NH₂, —NHCH₂CH₂CH₂NHCH₂CH₂CH₂CH₂NHCH₂CH₂CH₂NH₂, —NHCH₂CH₂NHCH₂CH₂CH₂CH₂NH₂, —NHCH₂CH₂NHCH₂CH₂CH₂NHCH₂CH₂CH₂NH₂, or —NHCH₂CH₂NH(CH₂CH₂NH)_{d}CH₂CH₂NH₂, where d is from 0 to 50.

In one aspect, the amine-modified natural polymer can include an aryl group having one or more amino groups directly or indirectly attached to the aromatic group. Alternatively, the amino group can be incorporated in the aromatic ring. For example, the aromatic amino group is a pyrrole, an isopyrrole, a pyrazole, imidazole, a triazole, or an indole. In another aspect, the aromatic amino group includes the isoimidazole group present in histidine. In another aspect, the biodegradable polyamine can be gelatin modified with ethylenediamine.

In another aspect, the polycation can be a polycationic micelle or mixed micelle formed with cationic surfactants. The cationic surfactant can be mixed with nonionic surfactants to create micelles with variable charge densities. The micelles are polycationic by virtue of the hydrophobic interactions that form a polyvalent micelle. In one aspect, the micelles have a plurality of amino groups capable of reacting with the activated ester groups present on the polyanion.

Examples of nonionic surfactants include the condensation products of a higher aliphatic alcohol, such as a fatty alcohol, containing about 8 to about 20 carbon atoms, in a straight or branched chain configuration, condensed with about 3 to about 100 moles, preferably about 5 to about 40 moles, most preferably about 5 to about 20 moles of ethylene oxide. Examples of such nonionic ethoxylated fatty alcohol surfactants are the Tergitol™ 15-S series from Union Carbide and Brij™ surfactants from ICI. Tergitol™ 15-S Surfactants include C₁₁-C₁₅ secondary alcohol polyethyleneglycol ethers. Brij™ 97 surfactant is polyoxyethylene(10) oleyl ether; Brij™ 58 surfactant is polyoxyethylene(20) cetyl ether; and Brij™ 76 surfactant is polyoxyethylene(10) stearyl ether.

Another useful class of nonionic surfactants include the polyethylene oxide condensates of one mole of alkyl phenol containing from about 6 to 12 carbon atoms in a straight or branched chain configuration, with ethylene oxide. Examples of nonreactive nonionic surfactants are the Igepal™ CO and CA series from Rhone-Poulenc. Igepal™ CO surfactants include nonylphenoxy poly(ethyleneoxy)ethanols. Igepal™ CA surfactants include octylphenoxy poly(ethyleneoxy)ethanols.

Another useful class of hydrocarbon nonionic surfactants include block copolymers of ethylene oxide and propylene oxide or butylene oxide. Examples of such nonionic block copolymer surfactants are the Pluronic™ and Tetronic™ series of surfactants from BASF. Pluronic™ surfactants include ethylene oxide-propylene oxide block copolymers. Tetronic™ surfactants include ethylene oxide-propylene oxide block copolymers.

In other aspects, the nonionic surfactants include sorbitan fatty acid esters, polyoxyethylene sorbitan fatty acid esters and polyoxyethylene stearates. Examples of such fatty acid ester nonionic surfactants are the Span™, Tween™, and Myj™ surfactants from ICI. Span™ surfactants include C₁₂-C₁₈ sorbitan monoesters. Tween™ surfactants include poly(ethylene oxide) C₁₂-C₁₈ sorbitan monoesters. Myj™ surfactants include poly(ethylene oxide) stearates.

In one aspect, the nonionic surfactant can include polyoxyethylene alkyl ethers, polyoxyethylene alkyl-phenyl ethers, polyoxyethylene acyl esters, sorbitan fatty acid esters, polyoxyethylene alkylamines, polyoxyethylene alkylamides, polyoxyethylene lauryl ether, polyoxyethylene cetyl ether, polyoxyethylene stearyl ether, polyoxyethylene oleyl ether, polyoxyethylene octylphenyl ether, polyoxyethylene nonylphenyl ether, polyethylene glycol laurate, polyethylene glycol stearate, polyethylene glycol distearate, polyethylene glycol oleate, oxyethylene-oxypropylene block copolymer, sorbitan laurate, sorbitan stearate, sorbitan distearate, sorbitan oleate, sorbitan sesquioleate, sorbitan trioleate, polyoxyethylene sorbitan laurate, polyoxyethylene sorbitan stearate, polyoxyethylene sorbitan oleate, polyoxyethylene laurylamine, polyoxyethylene laurylamide, laurylamine acetate, hard beef tallow propylenediamine dioleate, ethoxylated tetramethyldecynediol, fluoroaliphatic polymeric ester, polyether-polysiloxane copolymer, and the like.

Examples of cationic surfactants useful for making cationic micelles include alkylamine salts and quaternary ammonium salts. Non-limiting examples of cationic surfactants include: the quaternary ammonium surfactants, which can have up to 26 carbon atoms include: alkoxylate quaternary ammonium (AQA) surfactants as discussed in U.S. Pat. No. 6,136,769; dimethyl hydroxyethyl quaternary ammonium as discussed in U.S. Pat. No. 6,004,922; dimethyl hydroxyethyl lauryl ammonium chloride; polyamine cationic surfactants as discussed in WO 98/35002, WO 98/35003, WO 98/35004, WO 98/35005, and WO 98/35006; cationic ester surfactants as discussed in U.S. Pat. Nos. 4,228,042, 4,239,660 4,260,529 and U.S. Pat. No. 6,022,844; and amino surfactants as discussed in U.S. Pat. No. 6,221,825 and WO 00/47708, specifically amido propyldimethyl amine (APA).

In one aspect, the polycation includes a polyacrylate having one or more pendant amino groups. For example, the backbone of the polycation can be derived from the polymerization of acrylate monomers including, but not limited to, acrylates, methacrylates, acrylamides, and the like. In one aspect, the polycation backbone is derived from polyacrylamide. In other aspects, the polycation is a block co-polymer, where segments or portions of the co-polymer possess cationic groups or neutral groups depending upon the selection of the monomers used to produce the co-polymer.

In other aspects, the polycation can be a dendrimer. The dendrimer can be a branched polymer, a multi-armed polymer, a star polymer, and the like. In one aspect, the dendrimer is a polyalkylimine dendrimer, a mixed amino/ether dendrimer, a mixed amino/amide dendrimer, or an amino acid dendrimer. In another aspect, the dendrimer is poly(amidoamine), or PAMAM. In one aspect, the dendrimer has 3 to 20 arms, wherein each arm comprises an amino group.

In one aspect, the polycation is a polyamino compound. In another aspect, the polyamino compound has 10 to 90 mole % primary amino groups. In a further aspect, the polycation polymer has at least one fragment defined by Formula I below

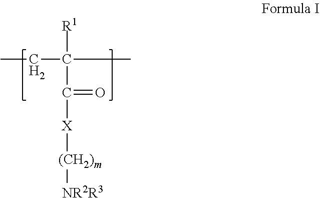

wherein R¹, R², and R³ are, independently, hydrogen or an alkyl group, X is oxygen or NR⁵, where R⁵ is hydrogen or an alkyl group, and m is from 1 to 10, or the pharmaceutically-acceptable salt thereof. In another aspect, R¹, R², and R³ are methyl and m is 2. Referring to formula I, the polymer backbone is composed of CH₂-CR¹ units with pendant—C(O)X(CH₂)ₘNR²R³ units. In one aspect, the polycation is the free radical polymerization product of a cationic primary amine monomer (3-amino-propyl methacrylate) and acrylamide, where the molecular weight is from 10 to 200 kd and possesses primary monomer concentrations from 5 to 90 mol %.

In another aspect, the polycation is a protamine. Protamines are polycationic, arginine-rich proteins that play a role in condensation of chromatin into the sperm head during spermatogenesis. As by-products of the fishing industry, commercially available protamines, purified from fish sperm, are readily available in large quantity and are relatively inexpensive. A non-limiting example of a protamine useful herein is salmine. Of the 32 amino acids in salmine, 21 are arginine (R). The guanidinyl group on the sidechain of R has a pKₐ of ^{˜}12.5, making salmine a densely charged polycation at physiologically relevant pH. It has a molecular mass of ^{˜}4,500 g/mol and a single negative charge at the carboxy terminus. In another aspect, the protamine is clupein.

In one aspect, the protamine can be derivatized with one or more crosslinkable groups described herein. For example, salmine can be derivatized to include one or more acrylate or methacrylate groups. An exemplary, non-limiting procedure for this embodiment is provided in the Examples. In this aspect, salmine has been derivatized on the C-terminal carboxylate with a single methacrylamide group to create a crosslinkable polycation.

In one aspect, the polycation is a natural polymer wherein one or more amine present on the natural polymer have been modified with a guanidine group. In another aspect, the polycation is a synthetic polymer containing one or more guanidinyl sidechains. For example, the polycation can be a synthetic polyguanidinyl polymer having an acrylate or methacrylate backbone and one or more guanidinyl sidechains. In another aspect, the polycation polymer has at least one fragment of the Formula VIII

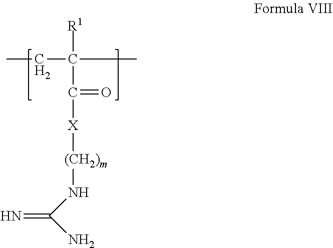

wherein R¹ is hydrogen or an alkyl group, X is oxygen or NR⁵, where R⁵ is hydrogen or an alkyl group, and m is from 1 to 10, or the pharmaceutically-acceptable salt thereof. In another aspect, R¹, R², and R³ are methyl and m is 2. Referring to formula VIII, the polymer backbone is composed of CH₂-CR¹ units with pendant —C(O)X(CH₂)ₘNC(NH)NH₂ units. An example synthetic polyguanidinyl polymer useful herein are shown below.

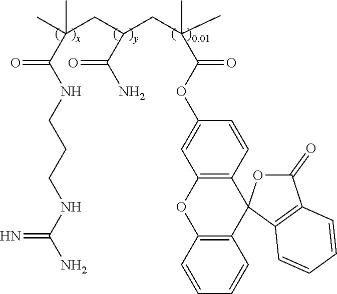

In another aspect, the synthetic polyguanidinyl polymer can be derivatized with one or more crosslinkable groups described herein. For example, one or more acrylate or methacrylate groups can be grafted onto the synthetic polyguanidinyl polymer. An example synthetic polyguanidinyl polymer with a methacrylate sidechain is shown below.

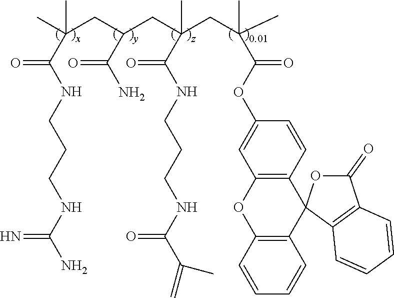

### Polyanions

Similar to the polycation, the polyanion can be a synthetic polymer or naturally-occurring. Examples of naturally-occurring polyanions include glycosaminoglycans such as condroitin sulfate, heparin, heparin sulfate, dermatan sulfate, keratin sulfate, and hyaluronic acid. In other aspects, acidic proteins having a net negative charge at neutral pH or proteins with a low pI can be used as naturally-occurring polyanions described herein. The anionic groups can be pendant to the polymer backbone and/or incorporated in the polymer backbone.

When the polyanion is a synthetic polymer, it is generally any polymer possessing anionic groups or groups that can be readily converted to anionic groups by adjusting the pH. Examples of groups that can be converted to anionic groups include, but are not limited to, carboxylate, sulfonate, boronate, sulfate, borate, phosphonate, or phosphate. Any cationic counterions can be used in association with the anionic polymers if the considerations discussed above are met.

In one aspect, the polyanion is a polyphosphate. In another aspect, the polyanion is a polyphosphate compound having from 5 to 90 mole % phosphate groups. For example, the polyphosphate can be a naturally-occurring compound such as, for example, DNA, RNA, or highly phosphorylated proteins like phosvitin (an egg protein), dentin (a natural tooth phosphoprotein), casein (a phosphorylated milk protein), or bone proteins (e.g. osteopontin).

Alternatively, the polyphosphoserine can be a synthetic polypeptide made by polymerizing the amino acid serine and then chemically phosphorylating the polypeptide. In another aspect, the polyphosphoserine can be produced by the polymerization of phosphoserine.

In one aspect, the polyphosphate can be produced by chemically or enzymatically phosphorylating a protein (e.g., natural serine- or threonine-rich proteins). In a further aspect, the polyphosphate can be produced by chemically phosphorylating a polyalcohol including, but not limited to, polysaccharides such as cellulose or dextran.

In another aspect, the polyphosphate can be a synthetic compound. For example, the polyphosphate can be a polymer with pendant phosphate groups attached to the polymer backbone and/or present in the polymer backbone. (e.g., a phosphodiester backbone).

In another aspect, the polyanion can be a micelle or mixed micelle formed with anionic surfactants. The anionic surfactant can be mixed with any of the nonionic surfactants described above to create micelles with variable charge densities. The micelles are polyanionic by virtue of the hydrophobic interactions that form a polyvalent micelle.

Other useful anionic surfactants include, but are not limited to, alkali metal and (alkyl)ammonium salts of: 1) alkyl sulfates and sulfonates such as sodium dodecyl sulfate, sodium 2-ethylhexyl sulfate, and potassium dodecanesulfonate; 2) sulfates of polyethoxylated derivatives of straight or branched chain aliphatic alcohols and carboxylic acids; 3) alkylbenzene or alkylnaphthalene sulfonates and sulfates such as sodium laurylbenzene-4-sulfonate and ethoxylated and polyethoxylated alkyl and aralkyl alcohol carboxylates; 5) glycinates such as alkyl sarcosinates and alkyl glycinates; 6) sulfosuccinates including dialkyl sulfosuccinates; 7) isothionate derivatives; 8)N-acyltaurine derivatives such as sodium N methyl-N-oleyltaurate); 9) amine oxides including alkyl and alkylamidoalkyldialkylamine oxides; and 10) alkyl phosphate mono or di-esters such as ethoxylated dodecyl alcohol phosphate ester, sodium salt. Representative commercial examples of suitable anionic sulfonate surfactants include, for example, sodium lauryl sulfate, available as TEXAPON™ L-100 from Henkel Inc., Wilmington, Del., or as POLYSTEP™ B-3 from Stepan Chemical Co, Northfield, Ill.; sodium 25 lauryl ether sulfate, available as POLYSTEP™ B-12 from Stepan Chemical Co., Northfield, Ill.; ammonium lauryl sulfate, available as STANDAPOL™ A from Henkel Inc., Wilmington, Del.; and sodium dodecyl benzene sulfonate, available as SIPONATE™ DS-10 from Rhone-Poulenc, Inc., Cranberry, N.J., dialkyl sulfosuccinates, having the tradename AEROSOL™ OT, commercially available from Cytec Industries, West Paterson, N.J.; sodium methyl taurate (available under the trade designation NIKKOL™ CMT30 from Nikko Chemicals Co., Tokyo, Japan): secondary alkane sulfonates such as Hostapur™ SAS which is a Sodium (C14-C17) secondary alkane sulfonates (alpha-olefin sulfonates) available from Clariant Corp., Charlotte, N.C.: methyl-2-sulfoalkyl esters such as sodium methyl-2-sulfo(C12-16)ester and disodium 2-sulfo(C12-C16) fatty acid available from Stepan Company under the trade designation ALPHASTE™ PC48; alkylsulfoacetates and alkylsulfosuccinates available as sodium laurylsulfoacetate (under the trade designation LANTHANOL™ LAL) and disodiumlaurethsulfosuccinate (STEPANMILD™ SL3), both from Stepan Company; alkylsulfates such as ammoniumlauryl sulfate commercially available under the trade designation STEPANOL™ AM from Stepan Company, and or dodecylbenzenesulfonic acid sold under BIO-SOFT® AS-100 from Stepan Chemical Co. In one aspect, the surfactant can be a disodium alpha olefin sulfonate, which contains a mixture of C12 to C16 sulfonates. In one aspect, CALSOFT™ AOS-40 manufactured by Pilot Corp. can be used herein as the surfactant. In another aspect, the surfactant is DOWFAX 2A1 or 2G manufactured by Dow Chemical, which are alkyl diphenyl oxide disulfonates.

Representative commercial examples of suitable anionic phosphate surfactants include a mixture of mono-, di- and tri-(alkyltetraglycolether)-o-phosphoric acid esters generally referred to as trilaureth-4-phosphate commercially available under the trade designation HOSTAPHA™ 340KL from Clariant Corp., as well as PPG-5 cetyl 10 phosphate available under the trade designation CRODAPHOS™ SG from Croda Inc., Parsipanny, N.J.

Representative commercial examples of suitable anionic amine oxide surfactants those commercially available under the trade designations AMMONYX™ LO, LMDO, and CO, which are lauryldimethylamine oxide, laurylamidopropyldimethylamine oxide, and cetyl amine oxide, all from Stepan Company.

In one aspect, the polyanion includes a polyacrylate having one or more pendant phosphate groups. For example, the polyanion can be derived from the polymerization of acrylate monomers including, but not limited to, acrylates, methacrylates, and the like. In other aspects, the polyanion is a block co-polymer, where segments or portions of the co-polymer possess anionic groups and neutral groups depending upon the selection of the monomers used to produce the co-polymer.

In one aspect, the polyanion includes two or more carboxylate, sulfate, sulfonate, borate, boronate, phosphonate, or phosphate groups.

In another aspect, the polyanion is a polymer having at least one fragment having the Formula XI

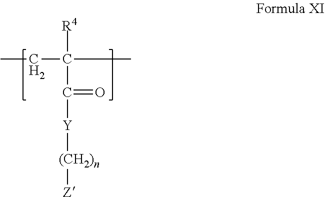

wherein
R⁴ is hydrogen or an alkyl group;
n is from 1 to 10;
Y is oxygen, sulfur, or NR³⁰,
R³⁰ is hydrogen, an alkyl group, or an aryl group;
Z′ is an anionic group or a group that can be converted to an anionic group, or the pharmaceutically-acceptable salt thereof.

In one aspect, Z′ in formula XI is carboxylate, sulfate, sulfonate, borate, boronate, a substituted or unsubstituted phosphate, or a phosphonate. In another aspect, Z′ in formula XI is sulfate, sulfonate, borate, boronate, a substituted or unsubstituted phosphate, or a phosphonate, and n in formulae XI is 2.

In another aspect, the polyanion is an inorganic polyphosphate possessing a plurality of phosphate groups (e.g., (NaPO₃)ₙ, where n is 3 to 10). Examples of inorganic phosphates include, but are not limited to, Graham salts, hexametaphosphate salts, and triphosphate salts. The counterion of these salts can be monovalent cations such as, for example, Na⁺, K⁺, and NH₄⁺.

In another aspect, the polyanion is phosphorylated sugar. The sugar can be a hexose or pentose sugar. Additionally, the sugar can be partially or fully phosphorylated. In one aspect, the phosphorylated sugar is inositol hexaphosphate.

### Crosslinkable Groups

In certain aspects, the polycations and polyanions can contain groups that permit crosslinking between the two polymers upon curing to produce new covalent bonds. The mechanism of crosslinking can vary depending upon the selection of the crosslinking groups. In one aspect, the crosslinking groups can be electrophiles and nucleophiles. For example, the polyanion can have one or more electrophilic groups, and the polycations can have one or more nucleophilic groups capable of reacting with the electrophilic groups to produce new covalent bonds. Examples of electrophilic groups include, but are not limited to, anhydride groups, esters, ketones, lactams (e.g., maleimides and succinimides), lactones, epoxide groups, isocyanate groups, and aldehydes. Examples of nucleophilic groups are presented below. In one aspect, the polycation and polyanion can crosslink with one another via a Michael addition. For example, the polycation can have one or more nucleophilic groups such as, for example, a hydroxyl or thiol group that can react with an olefinic group present on the polyanion.

In one aspect, the crosslinking group on the polyanion comprises an olefinic group and the crosslinking group on the polycation comprises a nucleophilic group that reacts with the olefinic group to produce a new covalent bond. In another aspect, the crosslinking group on the polycation comprises an olefinic group and the crosslinking group on the polyanion comprises a nucleophilic group that reacts with the olefinic group to produce a new covalent bond.

In other aspects, the crosslinkers present on the polycation and/or polyanion can form coordination complexes with transition metal ions. In one aspect, the polycation and/or polyanion can include groups capable of coordinating transition metal ions. Examples of coordinating sidechains are catechols, imidazoles, phosphates, carboxylic acids, and combinations. The rate of coordination and dissociation can be controlled by the selection of the coordination group, the transition metal ion, and the pH. Thus, in addition to covalent crosslinking as described above, crosslinking can occur through electrostatic, ionic, coordinative, or other non-covalent bonding. Transition metal ions such as, for example, iron, copper, vanadium, zinc, and nickel can be used herein. In one aspect, the transition metal is present in an aqueous environment at the application site.

In certain aspects, the in situ solidifying complex coacervate can also include a multivalent crosslinker. In one aspect, the multivalent crosslinker has two or more nucleophilic groups (e.g., hydroxyl, thiol, etc.) that react with crosslinkable groups (e.g., olefinic groups) present on the polycation and polyanion via a Michael addition reaction to produce a new covalent bond. In one aspect, the multivalent crosslinker is a di-thiol or tri-thiol compound.

### Reinforcing Components

The complex coacervates can optionally include a reinforcing component. The term “reinforcing component” is defined herein as any component that enhances or modifies one or more properties of the fluid complex coacervates described herein (e.g., cohesiveness, fracture toughness, elastic modulus, dimensional stability after curing, viscosity, etc.) of the in situ solidifying complex coacervate prior to or after the curing of the coacervate when compared to the same coacervate that does not include the reinforcing component. The mode in which the reinforcing component can enhance the mechanical properties of the coacervate can vary, and will depend upon the intended application of the coacervates as well as the selection of the polycation, polyanion, and reinforcing component. For example, upon curing the coacervate, the polycations and/or polyanions present in the coacervate can covalently crosslink with the reinforcing component. In other aspects, the reinforcing component can occupy a space or “phase” in the coacervate, which ultimately increases the mechanical properties of the coacervate. Examples of reinforcing components useful herein are provided below.

In one aspect, the reinforcing component is a polymerizable monomer. The polymerizable monomer entrapped in the complex coacervate can be any water soluble monomer capable of undergoing polymerization in order to produce an interpenetrating polymer network. In certain aspects, the interpenetrating network can possess nucleophilic groups (e.g., amino groups) that can react (i.e., crosslink) with the activated ester groups present on the polyanion. The selection of the polymerizable monomer can vary depending upon the application. Factors such as molecular weight can be altered to modify the solubility properties of the polymerizable monomer in water as well as the mechanical properties of the resulting coacervate,

The selection of the functional group on the polymerizable monomer determines the mode of polymerization. For example, the polymerizable monomer can be a polymerizable olefinic monomer that can undergo polymerization through mechanisms such as, for example, free radical polymerization and Michael addition reactions. In one aspect, the polymerizable monomer has two or more olefinic groups. In one aspect, the monomer comprises one or two actinically crosslinkable groups as defined above.

Examples of water-soluble polymerizable monomers include, but are not limited to, hydroxyalkyl methacrylate (HEMA), hydroxyalkyl acrylate, N-vinyl pyrrolidone, N-methyl-3-methylidene-pyrrolidone, allyl alcohol, N-vinyl alkylamide, N-vinyl-N-alkylamide, acrylamides, methacrylamide, (lower alkyl)acrylamides and methacrylamides, and hydroxyl-substituted (lower alkyl)acrylamides and -methacrylamides. In one aspect, the polymerizable monomer is a diacrylate compound or dimethacrylate compound. In another aspect, the polymerizable monomer is a polyalkylene oxide glycol diacrylate or dimethacrylate. For example, the polyalkylene can be a polymer of ethylene glycol, propylene glycol, or block copolymers thereof. In one aspect, the polymerizable monomer is polyethylene glycol diacrylate or polyethylene glycol dimethacrylate. In one aspect, the polyethylene glycol diacrylate or polyethylene glycol dimethacrylate has a Mₙ of 200 to 2,000, 400 to 1,500, 500 to 1,000, 500 to 750, or 500 to 600.

In certain aspects, the interpenetrating polymer network is biodegradable and biocompatible for medical applications. Thus, the polymerizable monomer is selected such that a biodegradable and biocompatible interpenetrating polymer network is produced upon polymerization. For example, the polymerizable monomer can possess cleavable ester linkages. In one aspect, the polymerizable monomer is hydroxypropyl methacrylate (HPMA), which will produce a biocompatible interpenetrating network. In other aspects, biodegradable crosslinkers can be used to polymerize biocompatible water soluble monomers such as, for example, alkyl methacrylamides. The crosslinker could be enzymatically degradable, like a peptide, or chemically degradable by having an ester or disulfide linkage. In another aspect, the reinforcing component can be a natural or synthetic fiber.

In other aspects, the reinforcing component can be a water-insoluble filler. The filler can have a variety of different sizes and shapes, ranging from particles (micro and nano) to fibrous materials. The selection of the filler can vary depending upon the application of the in situ solidifying complex coacervate.

The fillers useful herein can be composed of organic and/or inorganic materials. In one aspect, the nanostructures can be composed of organic materials like carbon or inorganic materials including, but not limited to, boron, molybdenum, tungsten, silicon, titanium, copper, bismuth, tungsten carbide, aluminum oxide, titanium dioxide, molybdenum disulphide, silicon carbide, titanium diboride, boron nitride, dysprosium oxide, iron (III) oxide-hydroxide, iron oxide, manganese oxide, titanium dioxide, boron carbide, aluminum nitride, or any combination thereof.

In certain aspects, the fillers can be functionalized in order to react (i.e., crosslink) with the polycation and/or polyanion. For example, the filler can be functionalized with amino groups or activated ester groups. In other aspects, it is desirable to use two or more different types of fillers. For example, a carbon nanostructure can be used in combination with one or more inorganic nanostructures.

In one aspect, the filler comprises a metal oxide, a ceramic particle, or a water insoluble inorganic salt. Examples of fillers useful herein include metallic, non-metallic, and inorganic nanoparticles, such as those commercially available from SkySpring Nanomaterials, Inc.

In one aspect, the filler is nanosilica. Nanosilica is commercially available from multiple sources in a broad size range. For example, aqueous Nexsil colloidal silica is available in diameters from 6-85 nm from Nyacol Nanotechnologies, Inc. Amino-modified nanosilica is also commercially available, from Sigma Aldrich for example, but in a narrower range of diameters than unmodified silica. Nanosilica does not contribute to the opacity of the coacervate, which is an important attribute of the adhesives and glues produced therefrom.

In certain aspects, the filler can be functionalized with one or more amino or activated ester groups. In this aspect, the filler can be covalently attached to the polycation or polyanion. For example, aminated silica can be reacted with the polyanion possessing activated ester groups to form new covalent bonds.

In other aspects, the filler can be modified to produce charged groups such that the filler can form electrostatic bonds with the coacervates. For example, aminated silica can be added to a solution and the pH adjusted so that the amino groups are protonated and available for electrostatic bonding.

In one aspect, the reinforcing component can be micelles or liposomes. In general, the micelles and liposomes used in this aspect are different from the micelles or liposomes used as polycations and polyanions for preparing the coacervate. The micelles and liposomes can be prepared from the nonionic, cationic, or anionic surfactants described above. The charge of the micelles and liposomes can vary depending upon the selection of the polycation or polyanion as well as the intended use of the coacervate. In one aspect, the micelles and liposomes can be used to solubilize hydrophobic compounds such pharmaceutical compounds. Thus, in addition to be used as adhesives, the adhesive complex coacervates described herein can be effective as a bioactive delivery device.

### Multivalent Cations

Complex coacervates can optionally contain one or more multivalent cations (i.e., cations having a charge of +2 or greater). In one aspect, the multivalent cation can be a divalent cation composed of one or more alkaline earth metals. For example, the divalent cation can be a mixture of Ca⁺² and Mg⁺². In other aspects, transition metal ions with a charge of +2 or greater can be used as the multivalent cation. The concentration of the multivalent cations can determine the rate and extent of coacervate formation. Not wishing to be bound by theory, weak cohesive forces between particles in the fluid may be mediated by multivalent cations bridging excess negative surface charges. The amount of multivalent cation used herein can vary. In one aspect, the amount is based upon the number of anionic groups and cationic groups present in the polyanion and polycation.

### Preparation of Complex Coacervates

The synthesis of complex coacervates described herein can be performed using a number of techniques and procedures. In one aspect, the polycation and polyanion are mixed as dilute solutions. Upon mixing, when the polycation and polyanion associate they condense into a fluid/liquid phase at the bottom of a mixing chamber (e.g., a tube) to produce a condensed phase. The condensed phase (i.e., fluid complex coacervate) is separated and used as the in situ solidifying complex coacervate.

In one aspect, an aqueous solution of polycation is mixed with an aqueous solution of polyanion such that the positive/negative charge ratio of the polycation to the polyanion is from 4 to 0.25, 3 to 0.25, 2 to 0.25, 1.5 to 0.5, 1.10 to 0.95, 1 to 1. Depending upon the number of charged groups on the polycation and polyanion, the amount of polycation and polyanion can be varied in order to achieve specific positive/negative charge ratios. The in situ solidifying complex coacervate contains water, wherein the amount of water is from 20% to 80% by weight of the composition.

The pH of the solution containing the polycation, polyanion, and the monovalent salt can vary in order to optimize complex coacervate formation. In one aspect, the pH of the composition containing the in situ solidifying complex coacervate is from 6 to 9, 6.5 to 8.5, 7 to 8, or 7 to 7.5. In another aspect, the pH of the composition is 7.2 (i.e., physiological pH).

The amount of the monovalent salt that is present in the in situ solidifying complex coacervate can vary depending upon the concentration of the monovalent salt in the environment at which the in situ solidifying complex coacervate is introduced. In general, the concentration of the monovalent salt in the complex coacervate is greater than the concentration of the monovalent salt in the environment. For example, the concentration of Na and KCl under physiological conditions is about 150 mM. Therefore, if the in situ solidifying complex coacervate is to be administered to a human subject, the concentration of the monovalent salt present in the in situ solidifying complex coacervate would be greater than 150 mM. In one aspect, the monovalent salt that is present in the in situ solidifying complex coacervate is at a concentration from 0.5 M to 2.0 M. In another aspect, the concentration of the monovalent salt is 0.5 to 1.8, 0.5 to 1.6, 0.5 to 1.4, or 0.5 to 1.2. In another aspect, the concentration of the monovalent salt in the complex coacervate is 1.5 to 2, 1.5 to 3, 1.5 to 4, 1.5 to 5, 1.5 to 6, 1.5 to 7, 1.5 to 8, 1.5 to 9 or 1.5 to 10 times greater than the concentration of the monovalent salt in the aqueous environment.

In one aspect, the monovalent salt can be sodium chloride or potassium chloride or a mixture. In other aspects, the in situ solidifying complex coacervate can be formulated in hypertonic saline solutions that can be used for parenteral or intravenous administration or by injection to a subject. In one aspect, the in situ solidifying complex coacervate can be formulated in Ringer's dextrose, dextrose and sodium chloride, lactated Ringer's, or other buffered saline solutions that can be safely administered to a subject, wherein the saline concentration has been adjusted so that it is greater than saline concentration at physiological conditions

### Crosslinkable Biomaterials

Examples of suitable crosslinkable biomaterials include multicomponent compositions which crosslink in situ to form a biocompatible polymeric matrix. For example, the crosslinkable biomaterial can comprise a first precursor molecule and a second precursor molecule. “Precursor molecule”, as used herein, generally refers to a molecule present in the crosslinkable biomaterial which interacts with (e.g., crosslinks with) other precursor molecules of the same or different chemical composition in the crosslinkable biomaterial to form a biocompatible polymeric matrix. Precursor molecules can include monomers, oligomers and polymers which can be crosslinked covalently and/or non-covalently.

The multiple components of the composition (e.g., the first precursor molecule and the second precursor molecule) can be combined prior to injection, can be present in two or more separate solutions which are combined during the injection (e.g., by mixing within a delivery device used to inject the material), or can be present in two or more separate solutions which are individually injected into the LAA.

In some embodiments, the crosslinkable biomaterial comprises a first precursor molecule present in a first solution and a second precursor molecule present in a second solution. In one embodiment, the two solutions are combined during the course of injection (e.g., by mixing within a delivery device used to inject the material). In another embodiment, the two solutions are individually injected into the LAA, and combine in situ. In these cases, the two solutions can be injected simultaneously or sequentially. Depending on the mechanism of crosslinking, an accelerator (e.g., a pH modifying agent or radical initiator) can be added and/or an external stimulus (e.g., UV irradiation) can be applied to ensure uniform and rapid curing of the crosslinkable biomaterial to form a biocompatible matrix. In cases where an accelerator is added, the accelerator can be incorporated into one or more of the solutions containing a precursor molecule prior to injection. The accelerator can also be present in a solution which does not contain a precursor molecule. This accelerator solution can then be injected simultaneously or sequentially with one or more solutions containing one or more precursor compounds to initiate formation of the biocompatible polymeric matrix.

Suitable precursor molecules can be selected in view of the desired properties of the fluid biomaterial and resultant biocompatible polymeric matrix. In some cases, the crosslinkable biomaterial comprises one or more oligomeric or polymeric precursor molecules. For example, precursor molecules can include, but are not limited to, polyether derivatives, such as poly(alkylene oxide)s or derivatives thereof, polysaccharides, peptides, and polypeptides, poly(vinyl pyrrolidinone) (“PVP”), poly(amino acids), and copolymers thereof.

The precursor molecules can further comprise one or more reactive groups. Reactive groups are chemical moieties in a precursor molecule which are reactive with a moiety (such as a reactive group) present in another precursor molecule to form one or more covalent and/or non-covalent bonds. Examples of suitable reactive groups include, but are not limited to, active esters, active carbonates, aldehydes, isocyanates, isothiocyanates, epoxides, alcohols, amines, thiols, maleimides, groups containing one or more unsaturaturated C—C bonds (e.g., alkynes, vinyl groups, vinylsulfones, acryl groups, methacryl groups, etc.), azides, hydrazides, dithiopyridines, N-succinimidyl, and iodoacetamides. Suitable reactive groups can be incorporated in precursor molecules to provide for crosslinking of the precursor molecules.

In some embodiments, one or more of the precursor molecules comprises a poly(alkylene oxide)-based oligomer or polymer. Poly(alkylene oxide)-based oligomer and polymers are known in the art, and include polyethylene glycol (“PEG”), polypropylene oxide (“PPO”), polyethylene oxide-co-polypropylene oxide (“PEO-PPO”), co-polyethylene oxide block or random copolymers, poloxamers, meroxapols, poloxamines, and polyvinyl alcohol (“PVA”). Block copolymers or homopolymers (when A=B) may be linear (AB, ABA, ABABA or ABCBA type), star (AₙB or BAₙC, where B is at least n-valent, and n is an integer of from 3 to 6) or branched (multiple A's depending from one B). In certain embodiments, the poly(alkylene oxide)-based oligomer or polymer comprises PEG, a PEO-PPO block copolymer, or combinations thereof.

In some embodiments, one or more of the precursor molecules is defined by Formula I or Formula II

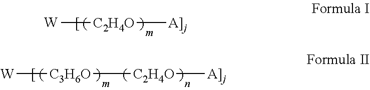

wherein
W is a branch point;
A is a reactive group (e.g., a nucleophilic group or a conjugated unsaturated group);
m and n are integers of from 1 to 500 (e.g., an integers of from 1 to 200); and
j is an integer greater than 2 (e.g., an integer of from 2 to 8).

In some embodiments, one or more of the precursor molecules comprises a biomacromolecule. The biomacromolecule can be, for example, a protein (e.g., collagen) or a polysaccharide. Examples of suitable polysaccharides include cellulose and derivatives thereof, dextran and derivatives thereof, hyaluronic acid and derivatives thereof, chitosan and derivatives thereof, alginates and derivatives thereof, and starch or derivatives thereof. Polysaccharides can derivatized by methods known in art. For example, the polysaccharide backbone can be modified to influence polysaccharide solubility, hydrophobicity/hydrophilicity, and the properties of the resultant biocompatible polymeric matrix formed from the polysaccharide (e.g., matrix degradation time). In certain embodiments, one or more of the precursor molecules comprises a biomacromolecule (e.g., a polysaccharide) which is substituted by two or more (e.g., from about 2 to about 100, from about 2 to about 25, or from about 2 to about 15) reactive groups (e.g., a nucleophilic group or a conjugated unsaturated group).

In some cases, the crosslinkable biomaterial can comprise a first precursor molecule which comprises an oligomer or polymer having one or more first reactive groups, each first reactive group comprising one or more pi bonds, and a second precursor molecule comprises an oligomer or polymer having one or more second reactive groups, each second reactive group comprising one or more pi bonds. The first reactive group can be reactive (e.g., via a Click chemistry reaction) with the second reactive group, so as to form a covalent bond between the first precursor molecule and the second precursor molecule. For example, the first reactive group and the second reactive group undergo a cycloaddition reaction, such as a [3+2]cycloaddition (e.g., a Huisgen-type 1,3-dipolar cycloaddition between an alkyne and an azide) or a Diels-Alder reaction.

In some cases, the crosslinkable biomaterial can comprise a first precursor molecule which comprises an oligomer or polymer having one or more nucleophilic groups (e.g. amino groups, thiol groups hydroxy groups, or combinations thereof), and a second precursor molecule which comprises an oligomer or polymer having one or more conjugated unsaturated groups (e.g., vinyl sulfone groups, acryl groups, or combinations thereof). In such cases, the first precursor molecule and the second precursor molecule can react via a Michael-type addition reaction. Suitable conjugated unsaturated groups are known in the art, and include those moieties described in, for example, U.S. Patent Application Publication No. US 2008/0253987 to Rehor, et al., which is incorporated herein by reference in its entirety.

In certain embodiments, the crosslinkable biomaterial can comprise a first precursor molecule and a second precursor molecule. The first precursor molecule comprises a poly(alkylene oxide)-based oligomer or polymer having x nucleophilic groups, wherein x is an integer greater than or equal to 2 (e.g., an integer of from 2 to 8, or an integer of from 2 to 6). The poly(alkylene oxide)-based polymer can comprise, for example, poly(ethylene glycol). The nucleophilic groups can be selected from the group consisting of sulfhydryl groups and amino groups. The first precursor molecule can have a molecular weight of from about 1 kDa to about 10 kDa (e.g., from about 1 kDa to about 5 kDa). In some embodiments, the first precursor molecule comprises pentaerythritol poly(ethylene glycol)ether tetrasulfhydryl.

The second precursor molecule can comprises a biomacromolecule having y conjugated unsaturated groups, wherein y is an integer greater than or equal to 2 (e.g., an integer of from 2 to 100, or an integer of from 2 to 25). The biomacromolecule can comprise a polysaccharide, such as dextran, hyaluronic acid, chitosan, alginate, or derivatives thereof. The conjugated unsaturated groups can be selected from the group consisting of vinyl sulfone groups and acryl groups. The second precursor molecule can have a molecular weight of from about 2 kDa to about 250 kDa (e.g., from about 5 kDa to about 50 kDa). In some embodiments, the second precursor molecule comprises dextran vinyl sulfone.

In some embodiments, the in situ crosslinking of the precursor molecules takes place under basic conditions. In these embodiments, the crosslinkable biomaterial can further include a base to activate the crosslinking of the precursor molecules. A variety of bases comply with the requirements of catalyzing, for example, Michael addition reactions under physiological conditions without being detrimental to the patient's body. Suitable bases include, but are not limited to, tertiary alkyl-amines, such as tributylamine, triethylamine, ethyldiisopropylamine, or N,N-dimethylbutylamine. For a given composition (and mainly dependent on the type of precursor molecules), the gelation time can be dependant on the type of base and of the pH of the solution. Thus, the gelation time of the composition can be controlled and adjusted to the desired application by varying the pH of the basic solution.

In some embodiments, the base, as the activator of the covalent crosslinking reaction, is selected from aqueous buffer solutions which have their pH and pK value in the same range. The pK range can be between 9 and 13. Suitable buffers include, but are not limited to, sodium carbonate, sodium borate and glycine. In one embodiment, the base is sodium carbonate.

### Interpenetrating Polymer Networks (IPNs)

IPNs can combine aspects of the characteristics of component chain polymer networks in ways that are distinct from those obtained by copolymerization or polymer blending. Unlike polymer blends, in which highly immiscible polymers can undergo extensive phase separation, the incompatible polymers in the IPN cannot phase separate. IPN formation permits polymers with very different properties (for example, a hydrophilic polymer and a hydrophobic polymer), to be combined to form mechanically robust hydrogels and elastomers. Examples of IPNs which can possess suitable biocompatibility and biomaterials properties for use in conjunction with the methods described herein include porous cross-linked polymer structures can be generated within a semi-IPN by selective solvent extraction of the linear component. The hydrophilicity and associated biocompatibility of a cross-linked network of silicone rubber can be improved (without adversely affecting the mechanical properties of the rubber) by swelling the rubber with 2-hydoxy ethyl methacrylate and polymerizing it to form a sequential IPN on the rubber surface. Materials formed from a porous poly(caprolactone) framework and a 3D-printed hydrogel cross-linked with UV light have also been prepared in which the porous network mechanically reinforces the reinforces the hydrogel and the hydrogel provides the hydrophilicity for biocompatibility.

In some embodiments, the fluid biomaterial can comprise a composition that forms an IPN in situ by simultaneous polymerization or cross-linking. For example, the fluid biomaterial can comprise a one pot, injectable based on peptides and chitosan, covalently cross-linked with a N-hydroxy succinimide terminated poly(ethylene glycol). Similarly, one-pot injectable mixtures of aliphatic epoxy and hydroxy methacrylate monomers can be polymerized (e.g., by light-initiated polymerization) to form parallel simultaneous polymerization routes (cationic for the epoxy and free radical for the methacrylate) with possibly some chain transfer between the epoxy and hydroxy moieties.

### Other Components

The fluid biomaterials described above can further contain organic and/or inorganic additives, such as thixotropic agents, stabilizers for stabilization of the precursor molecules in order to avoid premature crosslinking, and/or fillers which can result in an increase or improvement in the mechanical properties (e.g., cohesive strength and/or elastic modulus) of the resultant biocompatible matrix. Examples of stabilizing agents include radical scavengers, such as butylated hydroxytoluene or dithiothreitol.

In some embodiments, a bioactive agent can be incorporated into the fluid biomaterial (and thus into the resultant biocompatible polymer matrix). The bioactive agent can be a therapeutic agent, prophylactic agent, diagnostic agent, or combinations thereof. In some cases, the fluid biomaterial (and thus the resultant biocompatible polymer matrix) comprises an agent that promotes infiltration of cells onto or into the biocompatible polymeric matrix. For example, the agent can be an agent that promotes endothelialization. Promoting endothelialization refers to promoting, enhancing, facilitating, or otherwise increasing the attachment of, and growth of, endothelial cells on a surface of the biocompatible polymeric matrix. Examples of suitable agents that promote endothelialization are known in the art, and include growth factors (e.g., VEGF, PDGF, FGF, PIGF and combinations thereof), extracellular matrix proteins (e.g., collagen), and fibrin. In some cases, the fluid biomaterial (and thus the resultant biocompatible polymer matrix) comprises an anticoagulant, such as warfarin or heparin. In these cases, the anticoagulant can be locally delivered by elution from the resultant biocompatible polymer matrix. In some cases, the fluid biomaterial (and thus the resultant biocompatible polymer matrix) comprises a contrast agent, such as gold, platinum, tantalum, bismuth, or combinations thereof to facilitate imaging of the fluid biomaterial (e.g., during injection) or the resultant biocompatible polymer matrix (e.g., to confirm complete occlusion of the LAA or monitor degradation of the biocompatible polymer matrix).

In certain embodiments, the fluid biomaterial (and thus the resultant biocompatible polymer matrix) comprises a silencing agent. The silencing agent can comprise any suitable agent (small molecule or biological) that can be locally released from the fluid biomaterial (and/or the biocompatible polymer matrix) and eliminates contractility of cardiac tissue in the walls of the LAA (e.g., by interrupting electrical signals, inducing apoptosis, etc.). The biocompatible polymer matrix can provide for localized, controlled release of the silencing agent to cardiac tissue in the walls of the LAA. For example, the biocompatible polymer matrix can provide for localized release of an effective amount of the silencing agent to eliminate contractility of cardiac tissue in the walls of the LAA over a period of at least 24 hours, at least 48 hours, at least 1 week, at least 2 weeks, at least 1 month, at least 2 months, at least 3 months, or longer.

In some embodiments, the silencing agent can comprise an apoptotic agent. The term “apoptotic agent” as used herein is defined as a drug, toxin, compound, composition, or biological entity which bestows and/or activates apoptosis, or programmed cell death, onto a cell. Examples of apoptotic agents include aclarubicin, apoptosis gene modulators, apoptosis regulators, arginine deaminase, clotrimazole, curacin A, etoposide, gemcitabine, ras inhibitors, ras-GAP inhibitor, and topotecan. Other known apoptotic agents include taxanes including docetaxel and paclitaxel, anthracyclines, cyclophosphamide, vinca alkaloids, cisplatin, carboplatin, 5-fluoro-uracil, gemcitabine, capecitabin, navelbine, zoledronate, venetoclax, and ABT-737.

### Occlusion Devices

The occlusion device can be any device sized to be positioned within the ostium of the LAA, and which includes at least an occluder portion.

In some embodiments, the occlusion device can further include an anchor portion operably coupled to the occluder portion. In these embodiments, the occlusion device is further structured such that when the occlusion device is positioned within the ostium of the LAA, the anchor portion extends into the internal volume of the LAA.

In some embodiments, the occlusion device further comprises a hub. In such embodiments, the occluder portion can comprise a proximal end and a distal end, the proximal end coupled to the hub. Optionally, the anchor portion can be coupled to the occluder portion by way of the hub.

The occluder portion can be configured to move between an occluder-deployed state (e.g., where the occluder portion has a cross-sectional dimension effective to occlude the ostium of the LAA) and an occluder-nondeployed state (e.g., where the occluder portion has a cross-sectional dimension effective to allow the occlusion device to be advanced through a suitable delivery catheter). Likewise, the anchor portion can be configured to be moved between an anchor-deployed state (e.g., where the anchor portion extends into the internal volume of the LAA) and an anchor-nondeployed state (e.g., where the anchor portion has a cross-sectional dimension effective to allow the occlusion device to be advanced through a suitable delivery catheter)

In some embodiments, the anchor portion can comprise a plurality of anchor segments, wherein each of the plurality of anchor segments extend distally beyond the occluder portion when the occluder portion is in the occluder-deployed state and the anchor portion is in the anchor-deployed state. Each of the anchor segments can comprise a structure configured to enhance purchase of the anchor portion within the biocompatible polymeric matrix, such as a loop portion, a helical portion, a fin portion, a barb portion, or any combination thereof.

In some embodiments, the occluder portion can comprise a tissue growth member extending between the proximal end and the distal end of the occluder portion. In some cases, the tissue growth member can comprise one or more layers formed from an expanded polytetrafluoroethylene (ePTFE). These layers can form a proximal surface of the tissue growth member (i.e., facing the left atrium when the occlusion device is positioned within the ostium of the LAA).

Examples of suitable occlusion devices include those described in International Application Publication Nos. WO 2020/254907, WO 2013/067188, WO 2010/081041, WO 2010/148246, and WO 2009/052432, each of which is hereby incorporated by reference in its entirety.

By way of example, referring now to FIGS. 2A-2B, example occlusion device 20 and a distal end portion of a delivery system 22 are shown. The occlusion device 20 may include frame components of an occluder portion 24 and an anchor portion 26, the occluder portion 24 also including a tissue growth member 28 attached thereto. Further, the anchor portion 26 may be hingably coupled to the occluder portion 24 such that the anchor portion 26 may be actuated, upon deployment of the occluder portion 24, between a deployed position and a non-deployed position (not shown) via an actuation mechanism at a handle (not shown) of the delivery system 22. With this arrangement, the occlusion device 20 and delivery system 22 may provide functionality of separating the steps of deploying the occluder portion 24 and the anchor portion 26, thereby, providing additional and enhanced functionality to the physician to properly position and implant the occlusion device 20 in the LAA.

As set forth, the occluder portion 24 may include an occluder material or a tissue growth member 28 attached thereto. The tissue growth member 28 may be a porous material, or other cell attaching material or substrate, configured to promote endothelization and tissue growth thereover. The tissue growth member 28 may extend over a proximal side of the medical device 20 and, particularly, over the occluder portion 24 and may extend over a portion of the anchor portion 26 and hinges coupling the anchor portion 26 to the occluder portion 24. As such, due to the shape of the frame components of the occluder portion 24, the tissue growth member 28 may include a proximal face that is generally convex to form an outer surface 40. The tissue growth member 28 may also include an inner surface 42 on its distal side that is generally concave shaped. In one embodiment, the tissue growth member 28 may extend primarily over an outside surface of frame components of the occluder portion 24 with a portion of the tissue growth member 28 extending on both the outside surface and the inside surface of the frame components of the occluder portion 24. In another embodiment, the tissue growth member 28 may extend primarily over both the outside surface and the inside surface of the frame components of the occluder portion 24 of the medical device 20. In another embodiment, the tissue growth member 28 may extend solely over the outside surface of the frame components of the occluder portion 24.

With respect to FIGS. 2B and 2C, the tissue growth member 28 may include one or more types of materials and/or layers. In one embodiment, the tissue growth member 28 may include a first material layer 30 and a second material layer 32. The first material layer 30 may primarily be an underside layer or base layer of the tissue growth member 28. The first material layer 30 may include porous and conformable structural characteristics. For example, the first material layer 30 may include a foam type material, such as, a polyurethane foam or any other suitable polymeric material, such as a polymer fabric, woven or knitted. The second material layer 32 may include one or more layers of, for example, an expanded polytetrafluoroethylene (ePTFE) material. The second material layer 32 may be attached to an outer surface of the first material layer 30 with, for example, an adhesive. In one embodiment, the second material layer 32 may include a first layer 32A, a second layer 32B, and a third layer 32C such that the first layer 32A may be directly attached to the first material layer 30 and the third layer 32C may be an outer-most layer covering the proximal side of the medial device 20 with the second layer 32B extending therebetween. The various layers of the second material layer 32 may be bonded together by adhesives and/or by a thermal bonding heat process or other appropriate processes known in the art. In one particular example, the outer-most layers, such as the second and third layers 32B, 32C, may be formed of an ePTFE material having an internodal distance (sometimes referred to as pore size) of approximately 70μιη to approximately 90μιη. The first layer 32A of the second material layer 32, adjacent the first material layer 30, may be formed of an ePTFE material having a reduced internodal distance relative to the second and third layers 32B, 32C. For example, the intemodal distance of the first layer 32A may be approximately IOμιη. This first layer 32A may be bonded or adhered to the first material layer 30 using an adhesive material. Any other suitable sized layers of ePTFE may be employed, such as ePTFE having an internodal distance up to about 250μιη. Further, there may be one or more additional layers, similarly sized to the first layer 32A, extending over a hub end 34 with flaps 36 (outlined with an “X” configuration) where the delivery system 22 interconnects with the medical device 20 (see FIG. 2A).

The second material layer 32 made of ePTFE effectively prevents the passage of blood, due to the small internodal distance and pore size of the first layer 32A, while the larger internodal distance of other layers (e.g., 32B and 32C) enable tissue in-growth and endothelization to occur. Additionally, the first material layer 30, being formed of a polyurethane foam, enables aggressive growth of tissue from the LAA wall into the tissue growth member 28 at the inside or concave side of the medical device 20. Further, the first material layer 30 provides an exposed shelf 38 on the outer surface 40 around the periphery and distal end portion of the tissue growth member 28, which promotes aggressive fibroblast and tissue growth to further initiate endothelization over the outer surface 40 of the second material layer 32. It is noted that the use of appropriate adhesive materials between the first material layer 30 and the next adjacent layer 32A may also serve to fill in the pores of the next adjacent layer 32A and further inhibit possible flow of blood through the tissue growth member 28. Additional layers of ePTFE may also be included to the second material layer 32 of the tissue growth member 28.

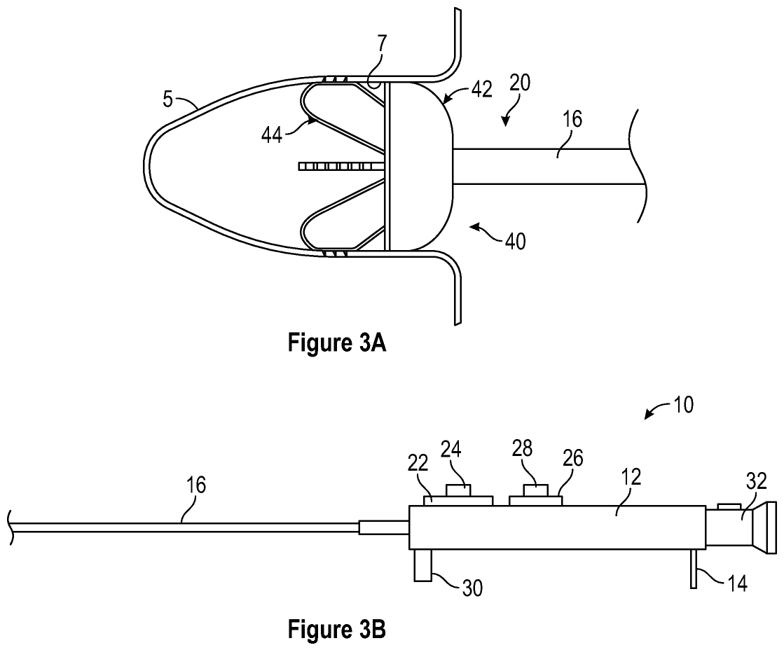

FIGS. 3A-3B illustrate another example occlusion device (40) that may be used in conjunction with the methods described herein. The occlusion device may be delivered by way of a delivery system 10 that includes a handle 12 with one or more actuators and a fluid port 14. In addition, the system 10 may include a catheter 16 with a catheter lumen extending longitudinally therethrough and attached to a distal end of the handle 12. Such a catheter lumen may coincide and communicate with a handle lumen as well as communicate with the fluid port 14.

The actuators associated with the handle may be configured to actuate or move an occlusion device 40 disposed within a distal portion 20 of the catheter 16 to deploy the occlusion device 40 from or within the distal portion 20 of the catheter 16, to capture (or recapture) the occlusion device 40 within the distal portion 20 of the catheter, or to do both. Such an occlusion device 40 can be interconnected to the handle 12 via tethers coils or other structures or elements (generally referred to as tethers herein for convenience) extending through the catheter 16 (tethers not shown). For example, the tethers can have a proximal end connected to the handle 12 and a distal end thereof connected to the occlusion device 40. The occlusion device 40 can be manipulated to be deployed and recaptured at different stages by controlling movement of the tether/coils (via the actuators) and controlling movement of the catheter 16.

The occlusion device 40, shown in deployed position in FIG. 3A (wherein the device is fully or at least substantially expanded), may include an occluder portion 42 and an anchor portion 44. As briefly noted above, the occlusion device 40 can be controlled to deploy in discrete stages with one stage being the deployment of the occluder portion 42 and another, discrete stage being deployment of the anchor portion 44. In this manner, a physician can first deploy the occluder portion 42, locate a preferable position and orientation for the occluder portion 42 in the LAA 5 and, once positioned and oriented satisfactorily, the physician can maintain such position while independently deploying the anchor portion 44. As such, the occluder portion 42 and the anchor portion 44 are configured to be deployed independent of one another as discrete, affirmative acts by a physician or operator of the system 10.

As previously noted, the handle 12 may include multiple actuators including a release mechanism 32. The release mechanism 32 is configured to release the occlusion device 40 from the tethers once the occlusion device 40 is anchored in the LAA 5 as will be described in further detail below. Other actuators may include a first actuator 22, a second actuator 24, a third actuator 26, a fourth actuator 28 and a fifth actuator 30 as shown in FIG. 3A. For example, the first actuator 22 and the second actuator 24 may be configured to control movement of the occluder portion 42 while the third actuator 26 and the fourth actuator 28 may be configured to control movement of the anchor portion 44. The fifth actuator 30 may be configured to control maneuverability of the distal portion 20 of the catheter 16 to negotiate tight corners and facilitate orientation when placing the medical device 40 in the LAA 5. It should be noted that, for example, the first actuator 22 and the second actuator 24 can be configured as, or to act as, a single actuator for the occluder portion 42. Likewise, the third actuator 26 and the fourth actuator 28 can be configured as, or to act as, a single actuator for the anchor portion 44.

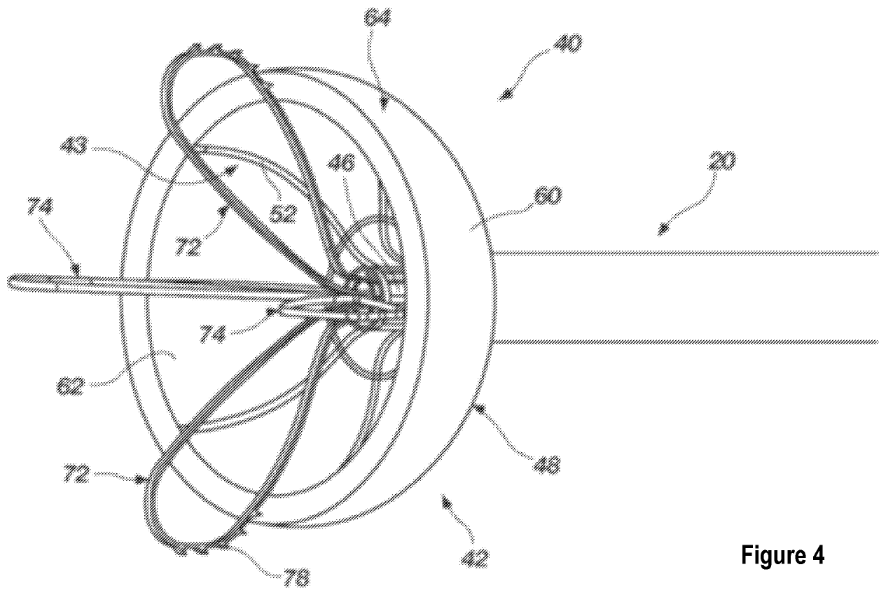

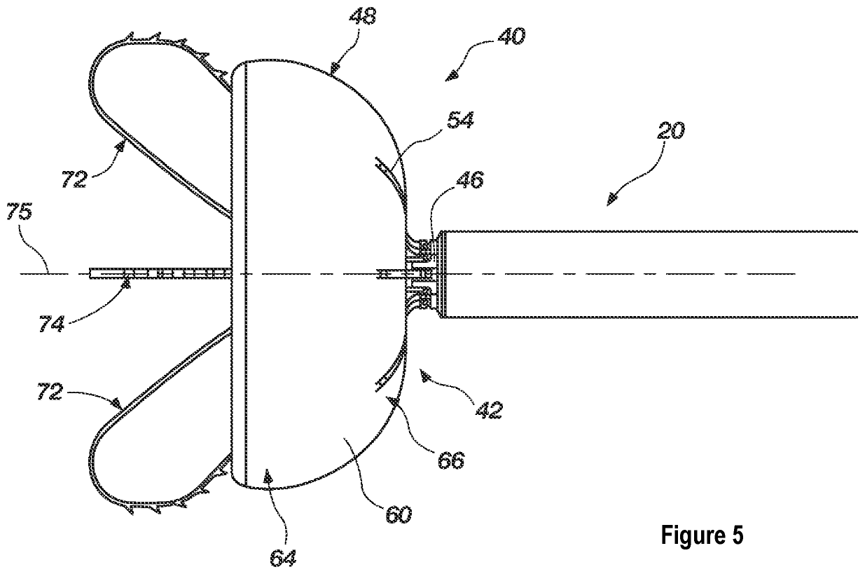

With reference to FIGS. 4 and 5, the occluder portion 42 may include an occluder frame 43 coupled to a hub 46 and a tissue growth member 48. The occluder frame 43 can include multiple occluder frame segments 50 extending radially and distally from the hub 46 generally in a spoke-like configuration. Such an occluder frame 43 is configured to assist in both expanding the tissue growth member 48 and in collapsing the tissue growth member 48. As such, each frame segment 50 may include an expander portion 52 and a collapser portion 54, wherein the expander portion 52 can include an overall length greater than that of the collapser portion 54. For example, each expander portion 52 may extend further radially, further distally, or both, as compared to a collapser portion 54.

Further, each frame segment 50 may include a clip 56 on each of the expander portion 52 and collapser portion 54. The clips 56 may be utilized to attach the tissue growth member 48 between the expander portion 52 and the collapser portion 54.

The tissue growth member 48 may include a porous structure configured to induce or promote tissue in-growth, or any other suitable structure configured to promote tissue in-growth. The tissue growth member 48 can include, for example, a body or a structure exhibiting a cup-like shape having an outer surface 60 and an inner surface 62. The outer surface 60 may include a distal surface portion 64 and a proximal surface portion 66. The outer surface distal surface portion 64 of the tissue growth member 48 can be sized and configured to be in direct contact with a tissue wall 7 within the LAA 5. In one embodiment, the tissue growth member 48 may be configured to self expand from a confined or constricted configuration to an expanded or deployed configuration. In one embodiment, the tissue growth member 48 may include a polymeric material, such as polyurethane foam. Other materials with desired porosity can also be used, such as felt, fabric, Dacron®, Nitinol braded wire, or polymeric or Nitinol felt. In the case of foam, such foam may be a reticulated foam, typically undergoing a chemical or heating process to open the pores within the foam as known by those of ordinary skill in the art. The foam may also be a non-reticulated foam. In one embodiment, the foam may include graded density or a graded porosity, as desired, and manipulated to expand in a desired shape when the frame member is moved to the expanded configuration.

In another embodiment, the tissue growth member 48 may include polyurethane foam with a skin structure on the inner surface 62, on the outer surface 60, or on both surfaces. For example, a skin structure may be formed on the inner surface 62 and be configured to inhibit blood from flowing through the tissue growth member 48, while the outer surface 60 of the tissue growth member may be configured to receive blood cells within its pores and induce tissue in-growth. In one embodiment, such a skin structure can include a layer of material, such as tantalum, sputtered to a surface of the tissue growth member 48. In another embodiment, the skin structure can include a polyurethane foam skin. Another example includes attaching expanded polytetrafluoroethylene (ePTFE) to the outer surface 60 or inner surface 62 of the tissue growth member 48, the ePTFE having minimal porosity to substantially inhibit blood flow while still allowing endothealization thereto.

In one embodiment, the anchor portion 44 may include a plurality of anchor segments and an anchor hub system 70. The anchor hub system 70 may be configured to be positioned and disposed within or adjacent to the hub 46. The plurality of anchor segments can include, for example, a first anchor segment 72 and a second anchor segment 74. Each of the first anchor segment 72 and the second anchor segment 74 may include a pedal or loop configuration (shown here in an expanded configuration), with, for example, two loop configurations for each of the first and second anchor segments 72 and 74, that are interconnected together via the anchor hub system 70. Each loop may be substantially oriented orthogonally with respect to an adjacent loop (i.e., in the embodiment shown in FIGS. 4 and 5, each loop of anchor component 72 being orthogonal to adjacent loops of anchor component 74). It is noted that, as used herein, the term “loop” does not require that a closed curve be formed of the component, but rather that a substantially closed curved or an open curve having a portion of the curve return on itself may also be considered as a “loop.”

While in the expanded configuration, each loop may extend distally of the occluder portion 42 and radially outward to a larger configuration than the anchor hub system 70. In other words, at least a portion of the anchor segments 72 and 74 extend distally beyond the distal-most portion of the occluder portion 42 and radially beyond the radial-most portion of the occluder portion as taken from a longitudinal axis 75 extending through the hub system 70. Each loop of an anchor segment 72 and 74 may also include engagement members or traction nubs 78 on an outer periphery of a loop configuration, the traction nubs 78 being sized and configured to engage and grab a tissue wall 7 and/or the biocompatible polymeric matrix within the LAA 5.

Each of the loop configurations of the first anchor segment 72, while in an expanded configuration, are substantially co-planar with each other and in a substantially flat configuration. Likewise, each of the loop configurations of the second anchor segment 74, while in an expanded configuration, are substantially co-planar with each other and in a substantially flat configuration. In one embodiment, the first anchor segment 72 may be attached to the second anchor segment 74 such that the loop configuration between the first and second anchor segments 72 and 74 are oriented substantially orthogonal with respect to each other. In other words, the plane in which the first anchor segment 72 is positioned or oriented is substantially orthogonal with respect to the plane of the second anchor segment 74. In other embodiments, there may be more than two anchor segments, in which case such anchor segments may or may not be oriented in a substantially orthogonal manner relative to each other.

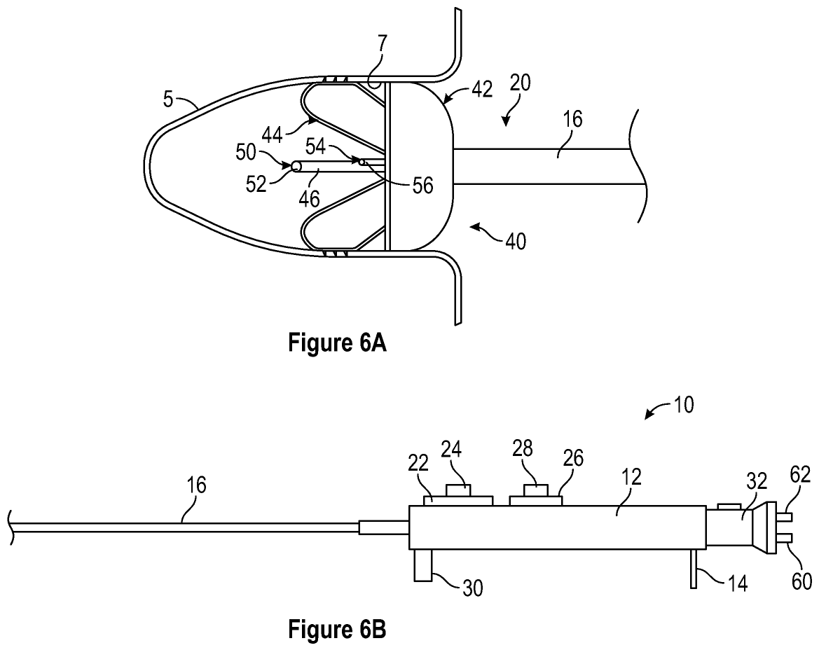

FIGS. 6A-6B illustrate another example occlusion device (40) that may be used in conjunction with the methods described herein. The occlusion device may be delivered by way of a delivery system 10 that includes a handle 12 with one or more actuators and a fluid port 14. In addition, the system 10 may include a catheter 16 with a catheter lumen extending longitudinally therethrough and attached to a distal end of the handle 12. Such a catheter lumen may coincide and communicate with a handle lumen as well as communicate with the fluid port 14.

The actuators associated with the handle may be configured to actuate or move an occlusion device 40 disposed within a distal portion 20 of the catheter 16 to deploy the occlusion device 40 from or within the distal portion 20 of the catheter 16, to capture (or recapture) the occlusion device 40 within the distal portion 20 of the catheter, or to do both. Such an occlusion device 40 can be interconnected to the handle 12 via tethers coils or other structures or elements (generally referred to as tethers herein for convenience) extending through the catheter 16 (tethers not shown). For example, the tethers can have a proximal end connected to the handle 12 and a distal end thereof connected to the occlusion device 40. The occlusion device 40 can be manipulated to be deployed and recaptured at different stages by controlling movement of the tether/coils (via the actuators) and controlling movement of the catheter 16.

The occlusion device 40, shown in deployed position in FIG. 3A (wherein the device is fully or at least substantially expanded), may include an occluder portion 42 and an anchor portion 44. As briefly noted above, the occlusion device 40 can be controlled to deploy in discrete stages with one stage being the deployment of the occluder portion 42 and another, discrete stage being deployment of the anchor portion 44. In this manner, a physician can first deploy the occluder portion 42, locate a preferable position and orientation for the occluder portion 42 in the LAA 5 and, once positioned and oriented satisfactorily, the physician can maintain such position while independently deploying the anchor portion 44. As such, the occluder portion 42 and the anchor portion 44 are configured to be deployed independent of one another as discrete, affirmative acts by a physician or operator of the system 10.

The occluder portion 42 comprises a proximal end and a distal end, the proximal end being coupled to a hub 46 having an injection lumen 50 passing axially therethrough. Injection lumen 50 terminates distally at an injection outlet 52. The delivery catheter can further include an injection channel 60 extending from the proximal end to the distal end and terminating in an outlet opening (not shown) which is fluidly connected to injection lumen 50 of the occlusion device. The injection channel and injection lumen provide a fluid flow path, allowing for the physician to inject a fluid biomaterial into the LAA after the occlusion device as been deployed within the ostium of the LAA.

Optionally, hub 46 can further comprise a second lumen 54 passing axially therethrough which is fluidly isolated from injection lumen 50. The second lumen 54 can terminate distally at a fluid inlet 56. In some cases, the injection outlet 52 can be separated from and distal to the fluid inlet 56. In these embodiments, the delivery catheter can further comprise an auxiliary lumen 62 fluidly isolated from the at least one injection channel and extending from the proximal end to the distal end and terminating in an auxiliary opening (not shown) which is fluidly connected to the second lumen of the occlusion device. The auxiliary lumen and second lumen provide a second fluid flow path, allowing for the physician to, for example, withdraw blood from the LAA after the occlusion device has been deployed within the ostium of the LAA. The auxiliary lumen and second lumen provide a second fluid flow path, allowing blood present in the LAA to flow from the LAA into the second lumen when a fluid biomaterial is injected into the LAA through the injection channel of the delivery catheter body and the injection lumen.

As previously noted, the handle 12 may include multiple actuators including a release mechanism 32. The release mechanism 32 is configured to release the occlusion device 40 from the tethers once the occlusion device 40 is anchored in the LAA 5 as will be described in further detail below. Other actuators may include a first actuator 22, a second actuator 24, a third actuator 26, a fourth actuator 28 and a fifth actuator 30 as shown in FIG. 3A. For example, the first actuator 22 and the second actuator 24 may be configured to control movement of the occluder portion 42 while the third actuator 26 and the fourth actuator 28 may be configured to control movement of the anchor portion 44. The fifth actuator 30 may be configured to control maneuverability of the distal portion 20 of the catheter 16 to negotiate tight corners and facilitate orientation when placing the medical device 40 in the LAA 5. It should be noted that, for example, the first actuator 22 and the second actuator 24 can be configured as, or to act as, a single actuator for the occluder portion 42. Likewise, the third actuator 26 and the fourth actuator 28 can be configured as, or to act as, a single actuator for the anchor portion 44.

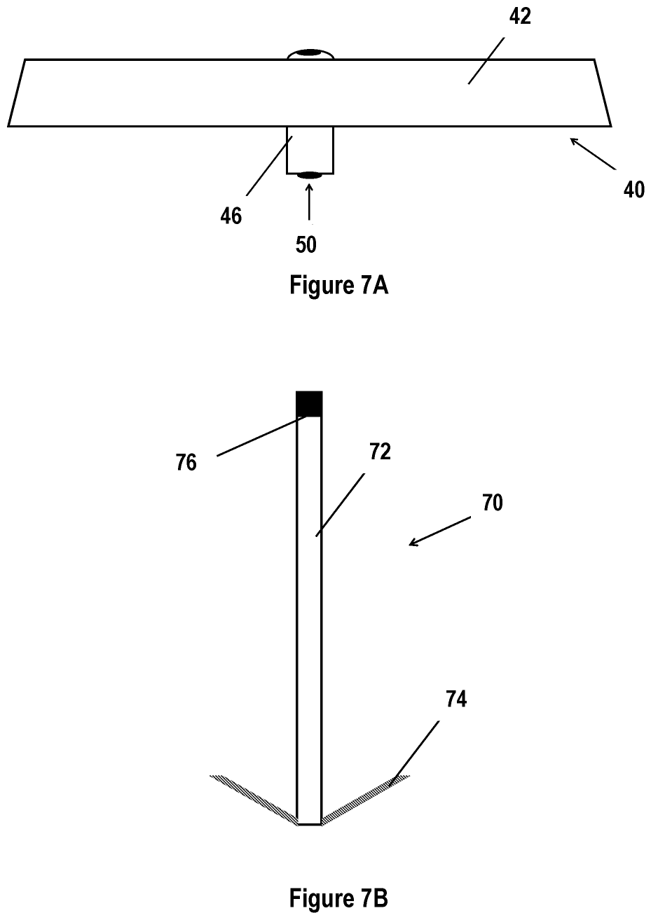

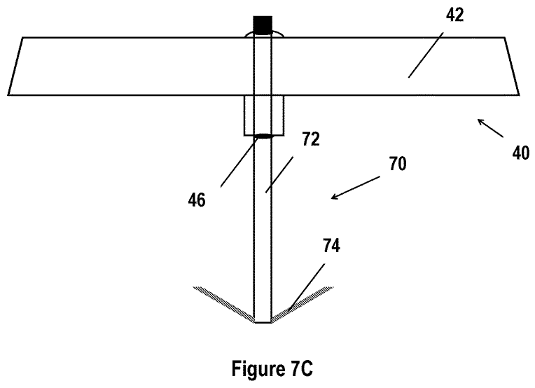

FIGS. 7A-7C illustrate another example occlusion device 40. As shown in FIG. 7A, the occlusion device can include an occluder portion 42 comprising a proximal end and a distal end, the proximal end coupled to a hub 46 having an injection lumen 50 passing axially therethrough. In these embodiments, the occlusion device does not include an integrated anchor portion, though one may optionally be present. As shown in FIG. 7B, the occlusion device can include a separate attachable anchor portion 70. The anchor portion can include one or more anchor segments 72 coupled to a fastener (76) that allows the anchor portion to be coupled (e.g., reversably) to the occlusion portion. As shown in FIG. 7C, in some embodiments, the anchor portion can be coupled to the occluder portion 42 by way of the hub 46 by means of fastener 76, such as by screwing the anchor portion to the hub. As with other embodiments described above, the anchor portion can be configured to move between an anchor-deployed state and an anchor-nondeployed state. For example, the anchor portion can include one or more barbs, fins, etc. (74) which can be deployed once the anchor portion has been advanced through the injection lumen and coupled to the occluder portion. The anchor portion (including the fastener) can be structured such that coupling of the anchor portion to the occlude portion seals any lumens which pass axially through the hub.

In other embodiments, the occlusion device can comprise an existing approved implant for occluding the LAA (e.g., the WATCHMAN™ LAAC Implant, the PLAATO (percutaneous left atrial appendage transcatheter occlusion) implant, or the Amplatzer device), optionally modified to include an anchor portion to allow them to more strongly interact and be secured by a biocompatible polymeric matrix formed in the LAA.

These example occlusion devices are discussed to exemplify some of the characteristics of suitable occlusion devices. One of ordinary skill in the art will appreciate that other occlusion devices having the characteristics above can also be used.

### Methods of Occluding the LAA

The fluid biomaterial and the occlusion device can be delivered to the LAA percutaneously (e.g., using a delivery catheter assembly and delivery system described below). The particular components and features of the delivery catheter assembly and delivery system can vary based on a number of factors, including the nature of the fluid biomaterial to be delivered. For example, the number of lumens in the delivery catheter assembly and/or the presence or absence of a mixing channel can be selected in view of the identity of the fluid biomaterial and/or the mechanism by which the fluid biomaterial cures.

Examples of suitable delivery systems include those described in International Application Publication Nos. WO 2020/254907, WO 2013/067188, WO 2010/081041, WO 2010/148246, and WO 2009/052432, each of which is hereby incorporated by reference in its entirety. Suitable delivery systems are also described above and exemplified, for example, in FIGS. 3A-3B and FIGS. 6A-6B.

In some embodiments, the delivery system can comprise a delivery catheter that includes a catheter body extending between a proximal end and a distal end, the catheter body comprising a wall structure that defines at least one injection channel extending from the proximal end to the distal end and terminating in an outlet opening; a handle coupled to the proximal end of the delivery catheter body, and the occlusion device operatively coupled to the handle and coupled to the distal end of the delivery catheter body. In embodiments where the occlusion device includes an injection lumen and the delivery catheter includes an injection channel, the injection channel can be fluidly connected to the injection lumen of the occlusion device. In embodiments where the occlusion device includes a second lumen and the delivery catheter includes an auxiliary lumen, the auxiliary lumen can be fluidly connected to the second lumen of the occlusion device.

Optionally, the delivery system can further comprise a sheath having a proximal end portion, a distal end portion having a distal tip, and a wall circumferentially enclosing a sheath lumen extending along an entire length of the sheath. The delivery catheter can be sized to be received and selectively advanceable within the sheath lumen such that the occlusion device can be passed through the sheath lumen to a position distal of the distal tip. Optionally, the sheath can further comprise at least one inflation channel within the wall of the sheath; and a balloon coupled to the distal end portion of the sheath and positioned in fluid communication with the at least one inflation channel of the sheath, the balloon enclosing an interior space. The wall of the delivery catheter body can define at least one outlet opening to provide fluid communication between the at least one inflation channel and the interior space of the balloon (allowing for inflation of the balloon using a suitable fluid).

Another example delivery catheter assembly can be based on the delivery catheter assembly described in International Application Publication No. WO 2019/099686, which is hereby incorporated by reference in its entirety. Briefly, the catheter assembly can comprise a first catheter body having a proximal end portion, a distal end portion having a distal tip, and a wall that circumferentially encloses a primary opening. The first catheter body can further comprise at least one inflation channel within the wall of the first catheter body. The primary opening of the first catheter body can extend along an entire length of the first catheter body. The catheter assembly can also comprise a first balloon coupled to the distal end portion of the first catheter body and positioned in fluid communication with the at least one inflation channel of the first catheter body. The first balloon can enclose an interior space, and the first catheter body can extend through the interior space of the first balloon in a proximal-to-distal direction such that at least the distal tip of the first catheter body is positioned distal of the first balloon.

The catheter assembly can also comprise a second catheter body partially received within the primary opening of, and selectively moveable relative to, the first catheter body. The second catheter body can include a proximal end portion, a distal end portion having a tip, and a wall that circumferentially encloses a primary opening. The second catheter body can further comprise at least one inflation channel within the wall of the second catheter body. The primary opening of the second catheter body can extend along an entire length of the second catheter body. The catheter assembly can further include a second balloon coupled to the distal end portion of the second catheter body and positioned in fluid communication with the at least one inflation channel of the second catheter body. The second balloon can enclose an interior space, and the second catheter body can extend through the interior space of the second balloon in the proximal-to-distal direction such that at least the distal tip of the second catheter body is positioned distal of the second balloon.

Additionally, the catheter assembly can comprise a third catheter body partially received within the primary opening of, and selectively moveable relative to, the second catheter body. The third catheter body can include a proximal end portion, a distal end portion, and a wall structure that defines at least one injection channel extending from the proximal end portion toward the distal end portion. The distal end portion of the third catheter body can further comprise at least one outlet opening positioned in fluid communication with the at least one injection channel. The third catheter body can be removable from the primary opening of the second catheter.

In these embodiments, the occlusion device can be positioned within the ostium of the LAA using a delivery system, wherein the delivery system comprises: a delivery catheter body extending between a proximal end and a distal end, a handle coupled to the proximal end of the delivery catheter body, and the occlusion device operatively coupled to the handle and coupled to the distal end of the delivery catheter body. The delivery system can be sized to be received within the primary opening of, and selectively moveable relative to, the second catheter body such that the occlusion device can be passed through the primary opening of the second catheter body to a position distal of the second balloon. The delivery system can be similar to those described in International Application Publication Nos. WO 2020/254907, WO 2013/067188, WO 2010/081041, WO 2010/148246, and WO 2009/052432, each of which is hereby incorporated by reference in its entirety. Suitable delivery systems are also described above and exemplified, for example, in FIGS. 3A-3B and FIGS. 6A-6B.

Such delivery catheters can be utilized in procedures where the occlusion device is positioned within the ostium of the LAA after injection of the fluid biomaterial into the LAA.

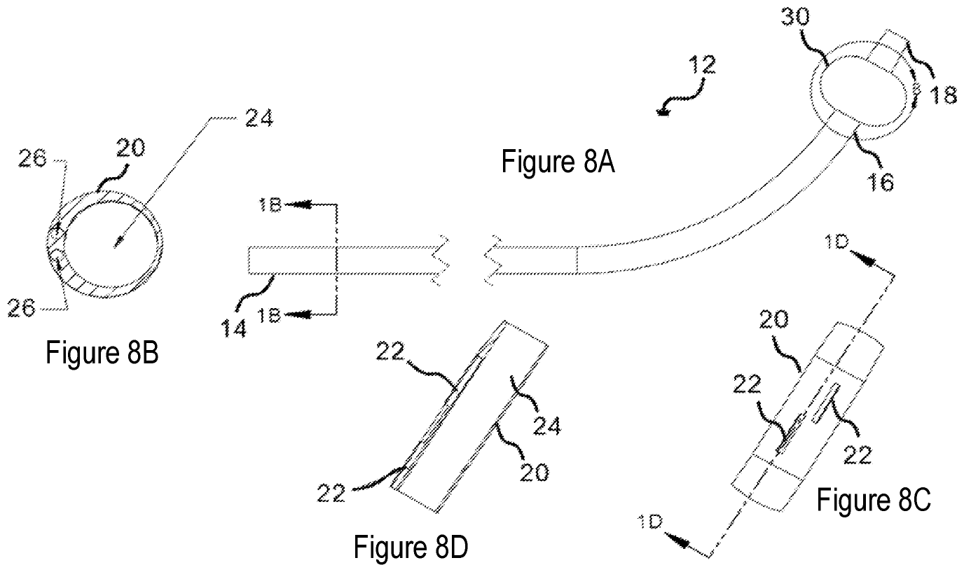

Referring now to FIGS. 8A-10D, the catheter assembly 10 can comprise three catheter bodies 12, 40, 70 and two balloons 30, 60 which cooperate with each other to deliver at least one fluid biomaterial and an occlusion device to the LAA of a subject's heart in a manner such that complete closure of the LAA is achieved.

In exemplary aspects, as shown in FIG. 8A, the catheter assembly 10 can comprise a first catheter body 12 that includes a proximal end portion 14, a distal end portion 16 having a distal tip 18, and a wall 20 that circumferentially encloses a primary opening 22. In these aspects, the primary opening 22 of the first catheter body 12 can extend along an entire length of the first catheter body. As shown in FIG. 8B, the first catheter body 12 can comprise at least one inflation channel 26 (optionally, a plurality of inflation channels, such as, for example, two inflation channels) within the wall 20 of the first catheter body. In further aspects, the catheter assembly 10 can comprise a first balloon 30 that can be coupled to the distal end portion 16 of the first catheter body 12 and positioned in fluid communication with the at least one inflation channel 26 of the first catheter body. In these aspects, the first balloon 30 can enclose an interior space 32. As shown in FIGS. 8C and 8D, the wall 20 of the first catheter body 12 can define at least one outlet opening 22 (optionally, a plurality of outlet openings) to provide fluid communication between the at least one inflation channel 26 and the interior space 32 of the first balloon 30. In some aspects, the each outlet opening can be in fluid communication with a respective inflation channel. In additional aspects, the plurality of outlet openings can be circumferentially spaced, axially spaced (in a distal or proximal direction), or both circumferentially and axially spaced in a staggered configuration In further aspects, the first catheter body 12 can extend through the interior space 32 of the first balloon 30 in a proximal-to-distal direction such that at least the distal tip 18 of the first catheter body 12 is positioned distal of the first balloon 30.

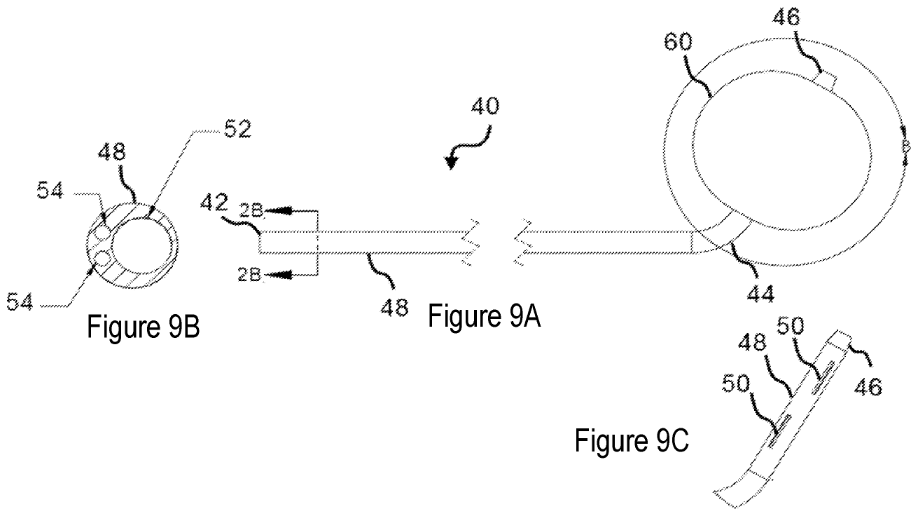

Referring to FIGS. 9A-9C, the catheter assembly 10 can comprise a second catheter body 40 that can be partially received within the primary opening 24 of, and selectively moveable relative to, the first catheter body 12. Optionally, in some aspects, it is contemplated that the second catheter body 40 can be selectively retractable relative to the first catheter body 12. In further optional aspects, it is contemplated that the first and second catheter bodies 12, 40 can be selectively lockable to maintain a desired position and orientation of the second catheter body 40 relative to the first catheter body 12. Optionally, in exemplary aspects, it is contemplated that the proximal ends of the first and second catheter bodies 12, 40 can be provided with Tuohy-type locking mechanisms as are known in the art (e.g., Tuohy-Borst adapters) to use friction to lock the first catheter body to the second catheter body. However, it is contemplated that any suitable locking mechanism as is known in the art can be used for this purpose. In additional aspects and as shown in FIG. 9A, the second catheter body 40 can include a proximal end portion 42, a distal end portion 44 having a distal tip 46, and a wall 48 that circumferentially encloses a primary opening 52 of the second catheter body 40. The primary opening 52 of the second catheter body 40 can extend along an entire length of the second catheter body. As shown in FIG. 9B, the second catheter body 40 can further comprises at least one inflation channel 54 (optionally, a plurality of inflation channels, such as for example, two inflation channels) within the wall 48 of the second catheter body 40.

In further aspects, the catheter assembly 10 can comprise a second balloon 60 that can be coupled to the distal end portion 44 of the second catheter body 40 and positioned in fluid communication with the at least one inflation channel 54 of the second catheter body. In these aspects, the second balloon 60 can enclose an interior space 62. As shown in FIG. 9C, the wall 48 of the second catheter body 40 can define at least one outlet opening 50 (optionally, a plurality of outlet openings) to provide fluid communication between the at least one inflation channel 54 and the interior space 62 of the second balloon. In some aspects, the each outlet opening 50 can be in fluid communication with a respective inflation channel 54. In additional aspects, the plurality of outlet openings 50 can be circumferentially spaced, axially spaced (in a distal or proximal direction), or both circumferentially and axially spaced in a staggered configuration. In further aspects, the second catheter body 40 can extend through the interior space 62 of the second balloon 60 in the proximal-to-distal direction such that at least the distal tip 46 of the second catheter body 40 is positioned distal of the second balloon 60.

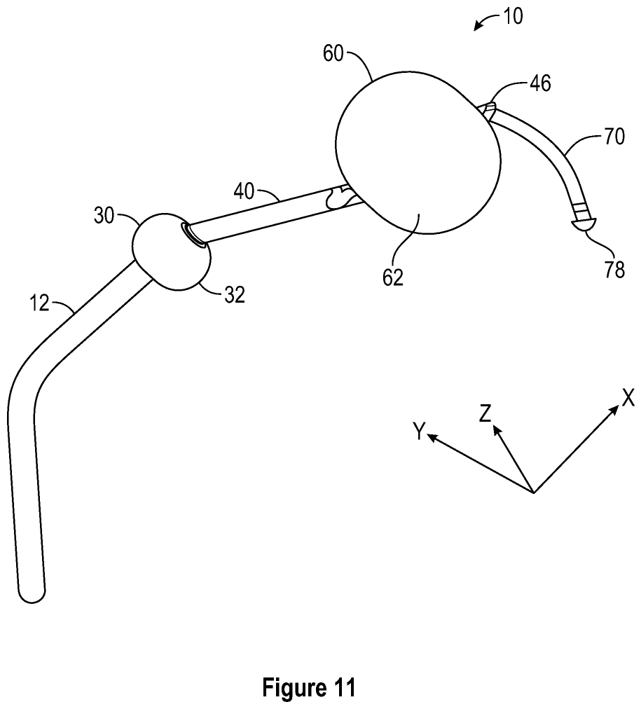

As depicted in FIG. 11, it is contemplated that when in an inflated position (e.g., a fully inflated position), the second balloon 60 can be larger (e.g., have a larger diameter) than the first balloon 30 (when the first balloon is also in an inflated or fully inflated position). Optionally, in exemplary aspects, it is contemplated that the maximum inflated diameter of the first balloon 30 can range from about 10 mm to about 20 mm or, more preferably, be about 15 mm. Optionally, in additional aspects, it is contemplated that the maximum inflated diameter of the second balloon 60 can range from about 30 mm to about 50 mm or from about 35 mm to about 45 mm or, more preferably, be about 40 mm. Optionally, in further exemplary aspects, it is contemplated that the first and second balloons are not spherical. For example, it is contemplated that each balloon can have an axial length (relative to the length of the catheter bodies) that is less than its maximum inflated diameter.

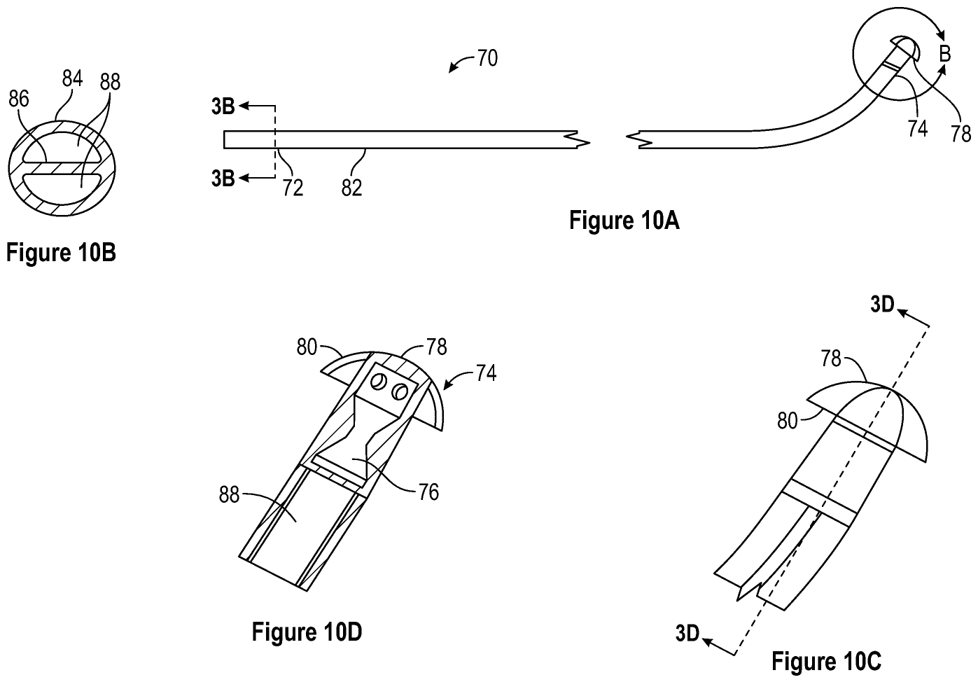

Referring to FIGS. 10A-10D, the catheter assembly 10 can comprise a third catheter body 70 that can be partially received within the primary opening 52 of, and selectively moveable relative to, the second catheter body 40. Optionally, in some aspects, the third catheter body 70 can be selectively retractable relative to the second catheter body 40. It is contemplated that, when the third catheter body is fully retracted, the third catheter body 70 can be fully received within the primary opening 52 of the second catheter body 40. In these aspects, as shown in FIG. 10A, the third catheter body 70 can have a proximal end portion 72, a distal end portion 74, and a wall structure 82 that defines at least one injection channel 88 extending from the proximal end portion 72 toward the distal end portion 74. Optionally, in some aspects, the at least one injection channel 88 can comprise a single injection channel. Alternatively, in some aspects, the at least one injection channel 88 of the third catheter body 70 can comprise a plurality of injection channels. For example, in some aspects, the at least one injection channel 88 of the third catheter body 70 can comprise first and second injection channels 88. In these aspects, as shown in FIG. 10B, the wall structure 82 of the third catheter body 70 can comprise an outer wall 84 and an inner wall 86 that extends between opposing portions of the outer wall to define the first and second injection channels 88. As shown in FIG. 10D, the distal end portion 74 of the third catheter body 70 can further comprise at least one outlet opening 90 positioned in fluid communication with the at least one injection channel 88. Optionally, in some aspects, the at least one outlet opening 90 of the distal end portion 74 of the third catheter body 70 can comprise a plurality of outlet openings. In further optional aspects, the distal end portion 74 of the third catheter body 70 can comprises a static mixing component 76 positioned between the at least one injection channel 88 and the at least one outlet opening 90, as shown in FIG. 10D. As used herein, the term “static mixing component” does not require any particular structural arrangement. Rather, the “static mixing component” includes any in-line structure that promotes mixing of the materials delivered through the respective injection channels 88 as further disclosed herein. Optionally, the static mixing component 76 can be a housed-elements type static mixer, a plate-type static mixer, or combinations thereof. More generally, it is contemplated that the static mixing component 76 can have a central receiving channel that provides for a variable flow pathway between the at least one injection channel 88 and the at least one outlet opening 90. Such a variable flow pathway can be created by projections and recesses (changes in diameter) of the interior surfaces of the static mixing component, as well as the presence of obstructions that prevent portions of the injected materials from following a consistent axial path in a proximal-to-distal direction. It is understood that when only a single injection channel 88 is provided, or in other situations where mixing of injectable components is unnecessary prior to delivery, it is possible to omit the static mixing component from the third catheter body 70.

In further aspects, the distal end portion 74 of the third catheter body 70 can have a distal tip 78 and a diaphragm 80 that is secured to the distal tip. In these aspects, the diaphragm 80 can extend outwardly from the distal tip 78. It is contemplated that the diaphragm 80 of the third catheter body 70 can occlude the primary opening 52 of the second catheter body 40 to prevent entry of material into the primary opening of the second catheter body in a distal-to-proximal direction. For example, it is contemplated that the diaphragm can be biased and/or deformable to a blocking position in which the outer diameter of the diaphragm is greater than the diameter of the primary opening 52 of the second catheter body. It is further contemplated that the diaphragm 80 can comprise a flexible material that is deformable as the third catheter body 70 exits the second catheter body (upon initial deployment) or is received within the second catheter body (upon retraction of the third catheter body), with the diaphragm blocking the entry of material into the second catheter body. In exemplary aspects, and as shown in FIGS. 10A-10C, the diaphragm can be secured to the distal tip 78 and have a convex outer surface extending circumferentially around the distal tip, with a proximal portion of the diaphragm at least partially overlapping with the outlet openings 90 (moving in a distal-to-proximal direction). As the third catheter body continues to move in a proximal direction within the primary opening of the second catheter body, the proximal surface of the diaphragm can contact the portions of the second catheter body to thereby movement of the diaphragm to a fully blocking position. Prior to complete receipt of the third catheter body 70 within the primary opening 52 of the second catheter body 40, it is contemplated that the injection of material through the outlet openings of the third catheter body can displace other fluid within the delivery site (e.g., LAA), with the displaced fluid flowing into the primary opening of the second catheter body.

It is contemplated that the first, second, and third catheter bodies can be formed from a variety of materials. The materials can be selected such that the first, second, and third catheter bodies have structural integrity sufficient to permit advancement of each catheter body as described herein and permit maneuvering and operation of each catheter body, while also permitting yielding and bending in response to encountered anatomical barriers and obstacles within the subject's body (e.g., within the vasculature). In exemplary aspects, the first, second, and/or third catheter bodies can be formed front a material or combination of materials, such as polymers, metals, and polymer-metal composites. In some aspects, soft durometer materials can be used to form all or part of the respective catheter body to reduce discomfort and minimize the risk of damage to the subject's vasculature (e.g., perforation). In some embodiments, the first, second, and/or third catheter bodies can be formed, in whole or in part, from a polymeric material. Examples of suitable plastics and polymeric materials include, but are not limited to, silastic materials and silicon-based polymers, polyether block amides (e.g., PEBAX®, commercially available from Arkema, Colombes, France), polynnides, polyurethanes, polyaniides (e.g., Nylon 6,6), polyvinylchlorides, polyesters e.g., HYTREL®, commercially available from DuPont, Wilmington, Del.), polyethylenes (PE), polyether ether ketone (PEEK), fluoropolymers such as polytetrafluoroethylene (PTFE), perfluoroalkoxy, fluorinated ethylene propylene, or blends and copolymers thereof. Examples of suitable metals which can form some or all of the first, second, and/or third catheter bodies include stainless steel (e.g., 304 stainless steel), nickel and nickel alloys (e.g., nitinol or MP-35N), titanium, titanium alloys, and cobalt alloys. In certain embodiments, each catheter body can comprise two different materials. Radiopaque alloys, such as platinum and titanium alloys, may also be used to fabricate, in whole or in part, the delivery catheter to facilitate real-time imaging during procedures performed using the delivery catheter. Optionally, the first, second, and/or third catheter bodies can be coated or treated with various polymers or other compounds in order to provide desired handling or performance characteristics, such as to increase lubricity. In certain embodiments, the first, second, and/or third catheter bodies can be coated with polytetrafluoroethylene (PTFE) or a hydrophilic polymer coating, such as poly(caprolactone), to enhance lubricity and impart desirable handling characteristics.

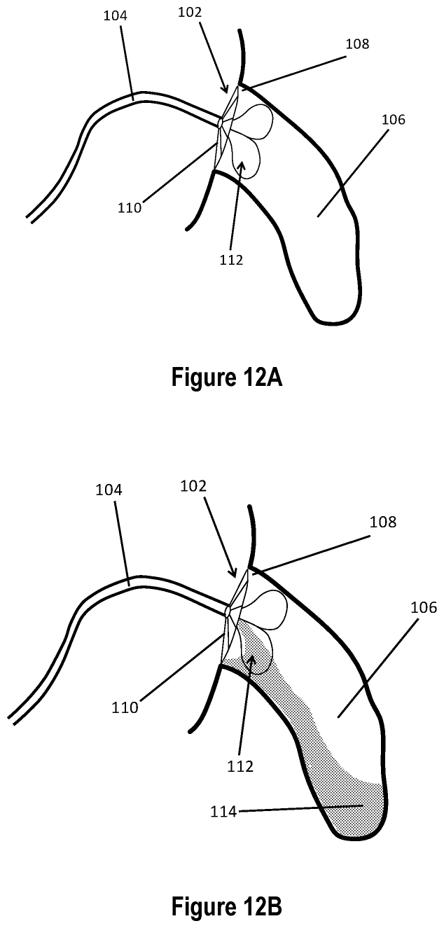

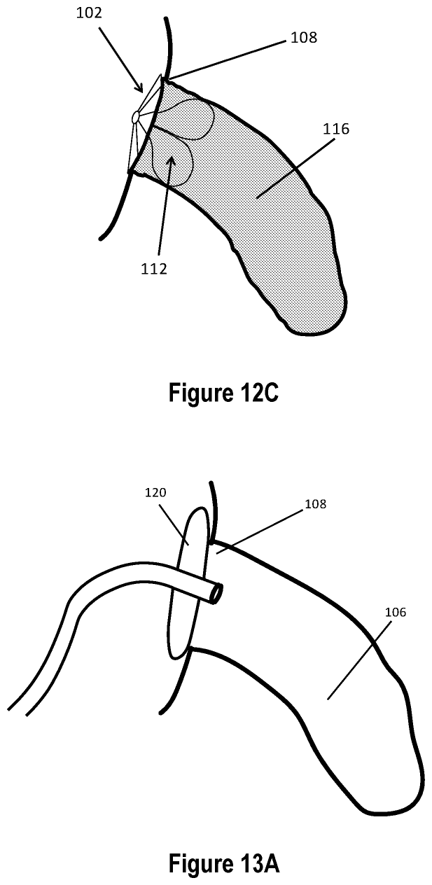

An example method for occluding the LAA of a patient is illustrated in FIGS. 12A-12C. These methods can comprise advancing a delivery system percutaneously through the patient's vasculature to reach the patient's right atrium. The delivery system can comprise a delivery catheter, wherein the delivery catheter comprises: a delivery catheter body extending between a proximal end and a distal end, the catheter body comprising a wall structure that defines at least one injection channel extending from the proximal end to the distal end and terminating in an outlet opening; a handle coupled to the proximal end of the delivery catheter body, and an occlusion device operatively coupled to the handle and coupled to the distal end of the delivery catheter body, wherein the occlusion device comprises an occluder portion comprising a proximal end and a distal end, the proximal end coupled to a hub having an injection lumen passing axially therethrough; and an anchor portion operably coupled to the occluder portion, wherein the outlet opening of the at least one injection channel is fluidly connected to the injection lumen of the occlusion device. Next, the delivery system can be advanced through an opening in the interatrial septum to reach the patient's left atrium. As shown in FIG. 12A, the occlusion device 102 can then be deployed within the ostium of the LAA 108 using the delivery catheter 104, such that the anchor portion 112 extends into the internal volume of the LAA (106). When deployed, the occluder portion 110 of the occlusion device 102 can substantially isolate the internal volume of the LAA (106) from the left atrium. Next, as shown in FIG. 12B, a fluid biomaterial 114 can be injected into the LAA through the injection channel of the delivery catheter body and the injection lumen of the occlusion device 102. The fluid biomaterial can exhibit a cure time following injection during which it remains flowable but after which it solidifies to form a biocompatible polymeric matrix that fills and occupies the internal volume of the LAA. The occlusion device can be retained within the ostium of the LAA until the cure time has elapsed and the fluid biomaterial has solidified to form the biocompatible polymeric matrix that fills and occupies the internal volume of the LAA. At this point, delivery catheter 104 can be withdrawn, leaving the occlusion device in place. As shown in FIG. 12C, because of the compliant nature of the fluid biomaterial, the resulting biocompatible polymeric matrix (116) can be interpenetrated by both the anchor portion (112) of the occlusion device and trabeculae present in the LAA (not shown for simplicity). In this way, the biocompatible polymeric matrix ensures that the occlusion device is retained within the ostium of the LAA 108. Further, the biocompatible matrix can function as an occlusive body, filling and occupying the internal volume of the LAA without adversely impacting cardiac function. In addition, the occlusion device can isolate the biocompatible polymeric matrix from blood present in the left atrium, provide a scaffold for endothelialization, of a combination thereof.

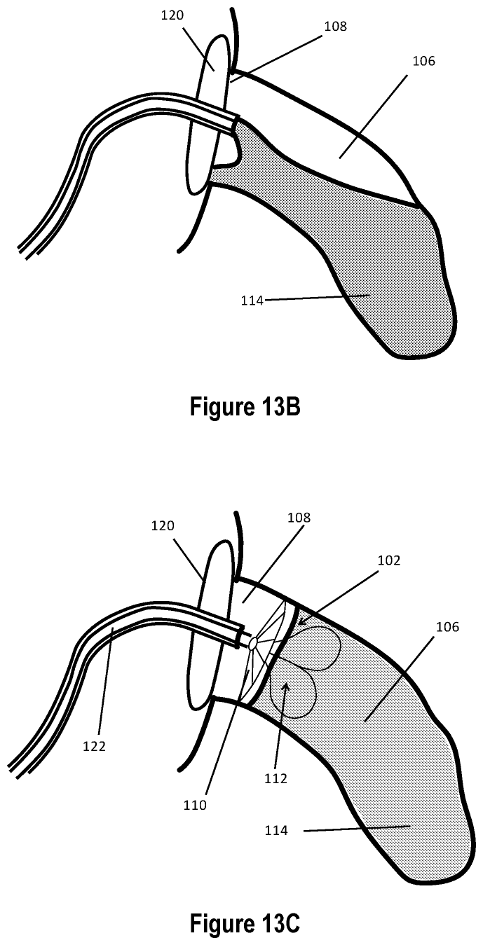

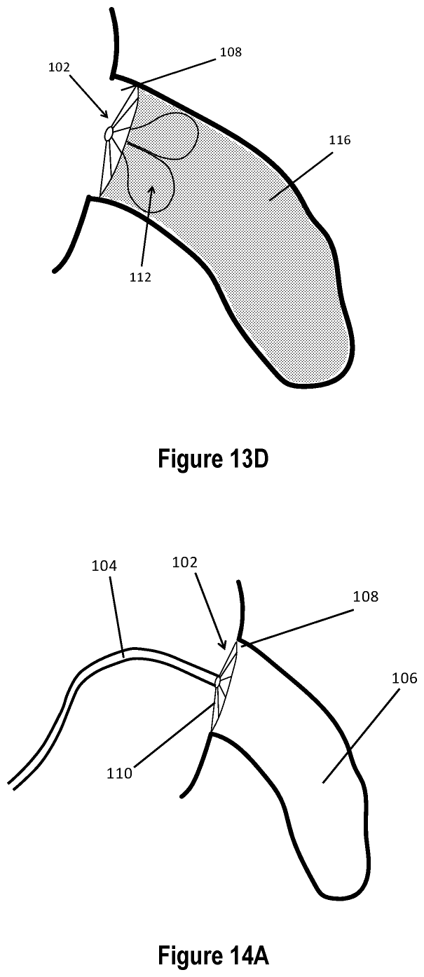

An alternative method for occluding the LAA of a patient is illustrated in FIGS. 13A-13D. These methods can comprise advancing a delivery catheter assembly percutaneously through the patient's vasculature to reach the patient's right atrium. The delivery catheter assembly can comprise: a first catheter body having a proximal end portion, a distal end portion having a distal tip, and a wall that circumferentially encloses a primary opening, wherein the first catheter body further comprises at least one inflation channel within the wall of the first catheter body, wherein the primary opening of the first catheter body extends along an entire length of the first catheter body; a first balloon coupled to the distal end portion of the first catheter body and positioned in fluid communication with the at least one inflation channel of the first catheter body, the first balloon enclosing an interior space, wherein the first catheter body extends through the interior space of the first balloon in a proximal-to-distal direction such that at least the distal tip of the first catheter body is positioned distal of the first balloon; a second catheter body partially received within the primary opening of, and selectively moveable relative to, the first catheter body, wherein the second catheter body has a proximal end portion, a distal end portion having a tip, and a wall that circumferentially encloses a primary opening, wherein the second catheter body further comprises at least one inflation channel within the wall of the second catheter body, wherein the primary opening of the second catheter body extends along an entire length of the second catheter body; a second balloon coupled to the distal end portion of the second catheter body and positioned in fluid communication with the at least one inflation channel of the second catheter body, the second balloon enclosing an interior space, wherein the second catheter body extends through the interior space of the second balloon in the proximal-to-distal direction such that at least the distal tip of the second catheter body is positioned distal of the second balloon; and a third catheter body partially received within the primary opening of, and selectively moveable relative to, the second catheter body, wherein the third catheter body has a proximal end portion, a distal end portion, and a wall structure that defines at least one injection channel extending from the proximal end portion toward the distal end portion, wherein the distal end portion of the third catheter body further comprises at least one outlet opening positioned in fluid communication with the at least one injection channel. Next, the delivery catheter assembly can be advanced through an opening in the interatrial septum to reach the patient's left atrium. The first balloon can then be inflated to anchor and secure the delivery catheter assembly within the left atrium. Next, the second catheter body can be advanced relative to the first catheter body to reach the LAA. As shown in FIG. 13A, the second balloon 120 can then be inflated to occlude the ostium of the LAA (108) of the patient. The third catheter body can then be advanced relative to the second catheter body. Next as shown in FIG. 13B, a fluid biomaterial 104 can be injected into the LAA through the injection channel of the third catheter body. The fluid biomaterial can exhibit a cure time following injection during which it remains flowable but after which it solidifies to form a biocompatible polymeric matrix that fills and occupies the internal volume of the LAA 106. Subsequently, the third catheter body can be removed from the primary opening of the second catheter. A delivery system sized to be received within the primary opening of, and selectively moveable relative to, the second catheter body can then be inserted into the primary opening of the second catheter body. The delivery system can comprise: a delivery catheter body extending between a proximal end and a distal end, a handle coupled to the proximal end of the delivery catheter body, and an occlusion device operatively coupled to the handle and coupled to the distal end of the delivery catheter body, wherein the occlusion device comprises an occluder portion and an anchor portion operably coupled to the occluder portion. As shown in FIG. 13C, the delivery system 122 can then be advanced within the primary opening of the second catheter body such that the occlusion device 102 is passed through the primary opening of the second catheter body to a position distal of the second balloon 120. The occlusion device 102 can then be deployed within the ostium of the LAA 108 such that the anchor portion 112 extends into the internal volume of the LAA 106. The occlusion device can be retained within the ostium of the LAA until the cure time has elapsed and the fluid biomaterial has solidified to form the biocompatible polymeric matrix. At this point, delivery catheter assembly can be withdrawn, leaving the occlusion device in place. As shown in FIG. 13D, because of the compliant nature of the fluid biomaterial, the resulting biocompatible polymeric matrix (116) can be interpenetrated by both the anchor portion (112) of the occlusion device and trabeculae present in the LAA (not shown for simplicity). In this way, the biocompatible polymeric matrix ensures that the occlusion device 102 is retained within the ostium of the LAA 108. Further, the biocompatible matrix can function as an occlusive body, filling and occupying the internal volume of the LAA without adversely impacting cardiac function. In addition, the occlusion device can isolate the biocompatible polymeric matrix from blood present in the left atrium, provide a scaffold for endothelialization, of a combination thereof.

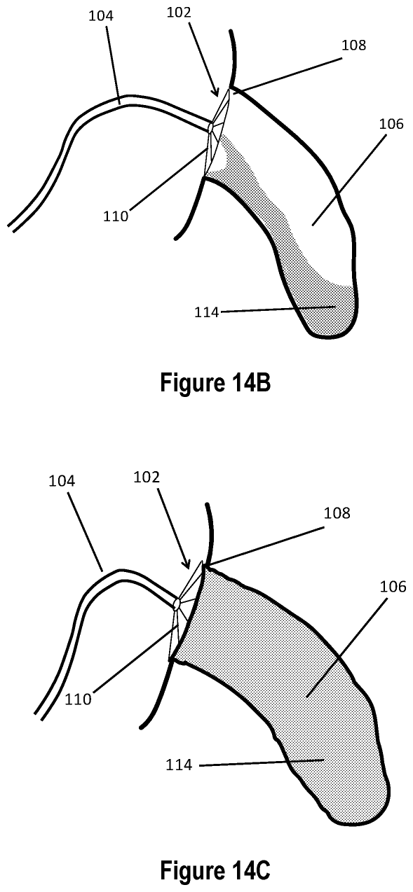

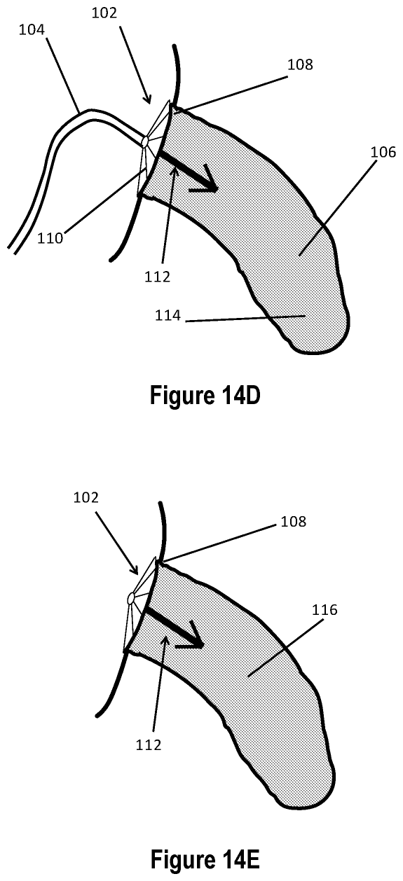

An alternative method for occluding the LAA of a patient is illustrated in FIGS. 14A-14E. These methods can comprise advancing a delivery system percutaneously through the patient's vasculature to reach the patient's right atrium. The delivery system can comprise a delivery catheter, wherein the delivery catheter comprises: a delivery catheter body extending between a proximal end and a distal end, the catheter body comprising a wall structure that defines at least one injection channel extending from the proximal end to the distal end and terminating in an outlet opening; a handle coupled to the proximal end of the delivery catheter body, and an occlusion device operatively coupled to the handle and coupled to the distal end of the delivery catheter body, wherein the occlusion device comprises an occluder portion comprising a proximal end and a distal end, the proximal end coupled to a hub having an injection lumen passing axially therethrough, wherein the outlet opening of the at least one injection channel is fluidly connected to the injection lumen of the occlusion device. Next, the delivery system can be advanced through an opening in the interatrial septum to reach the patient's left atrium. As shown in FIG. 14A, the occlusion device 102 can then be deployed within the ostium of the LAA 108 using the delivery catheter 104. When deployed, the occluder portion 110 of the occlusion device 102 can substantially isolate the internal volume of the LAA (106) from the left atrium. Next, as shown in FIGS. 14B and 14C, a fluid biomaterial 114 can be injected into the LAA through the injection channel of the delivery catheter body and the injection lumen of the occlusion device 102. The fluid biomaterial can exhibit a cure time following injection during which it remains flowable but after which it solidifies to form a biocompatible polymeric matrix that fills and occupies the internal volume of the LAA. As shown in FIG. 14D, an anchoring portion can then be advanced through the injection channel of the delivery catheter body and the injection lumen and coupling the anchoring portion 114 to the occlusion device 102. When the anchoring portion 112 is coupled to the occlusion device 102, the anchor portion 102 extends into the internal volume of the LAA 106. The occlusion device can be retained within the ostium of the LAA until the cure time has elapsed and the fluid biomaterial has solidified to form the biocompatible polymeric matrix that fills and occupies the internal volume of the LAA. At this point, delivery catheter 104 can be withdrawn, leaving the occlusion device in place. As shown in FIG. 14E, because of the compliant nature of the fluid biomaterial, the resulting biocompatible polymeric matrix (116) can be interpenetrated by both the anchor portion (112) of the occlusion device and trabeculae present in the LAA (not shown for simplicity). In this way, the biocompatible polymeric matrix ensures that the occlusion device is retained within the ostium of the LAA 108. Further, the biocompatible matrix can function as an occlusive body, filling and occupying the internal volume of the LAA without adversely impacting cardiac function. In addition, the occlusion device can isolate the biocompatible polymeric matrix from blood present in the left atrium, provide a scaffold for endothelialization, of a combination thereof.

In practicing the methods described herein, the delivery catheters/systems can be inserted into the vasculature of the patient (e.g., into the femoral vein), and advanced through the patient's vasculature, such that they reach the patient's left atrium. The LAA may be accessed through any of a variety of pathways as will be apparent to those of skill in the art. Trans-septal access can be achieved by introducing the delivery catheter/system into, for example, the femoral or jugular vein, and transluminally advancing the catheter into the right atrium. Radiographic imaging (e.g., single or biplanar flouroscopy, sonographic imaging, or combinations thereof) can be used to image the delivery catheter during the procedure and guide the distal end of the catheter to the desired site. As a result, in some cases, at least a portion of the delivery catheter can be formed to be at least partially radiopaque.

Once in the right atrium, a long hollow needle with a preformed curve and a sharpened distal tip can be advanced through the delivery catheter/sheath, and forcibly inserted through the fossa ovalis. A radiopaque contrast media can be injected through the needle to allow visualization and ensure placement of the needle in the left atrium, as opposed to being in the pericardial space, aorta, or other undesired location. Once the position of the needle in the left atrium is confirmed, the delivery catheter/sheath can be advanced over the needle through the septum and into the left atrium. Alternative approaches to the LAA are known in the art, and can include venous transatrial approaches such as transvascular advancement through the aorta and the mitral valve.

If desired, fluid (e.g., blood) present in the LAA can be removed following sealing of the LAA (e.g., with the occlusion device) but prior to injection of the fluid biomaterial. Optionally, the volume of blood removed from the LAA of the patient can be measured, and used to determine an appropriate amount of fluid biomaterial to be injected into the LAA of the patient. In another embodiment, diagnostic imaging and image analysis can be utilized to determine an appropriate amount of fluid biomaterial to be injected into the LAA of the patient. In some embodiments, the total volume of fluid biomaterial injected ranges from about 2 mL to about 15 mL (e.g., from 2 mL to about 10 mL, or from about 5 mL to about 15 mL). The fluid biomaterial can be injected via one or more lumens, for example, using a syringe, inflator, or other device fluidly connected to the one or more lumens. In certain embodiments, the fluid biomaterial can comprise a first precursor molecule present in a first solution and a second precursor molecule present in a second solution, wherein the first precursor molecule is reactive with the second precursor molecule to form a biocompatible polymeric matrix. In these embodiments, the first solution can be injected into a first lumen in the catheter and the second solution can be injected into a second lumen in the catheter. In one embodiment, the two solutions are combined during the course of injection via the delivery catheter (e.g., by mixing within a mixing channel within the delivery catheter). In another embodiment, the two solutions are individually (simultaneously or sequentially) injected into the LAA, and combine in situ to form a biocompatible polymeric matrix. In cases when the two solutions are simultaneously injected, the two solutions can be injected using, for example, a dual-barrel syringe or indeflator, wherein the first barrel contains the first solution and is fluidly connected to the first lumen, and the second barrel contains the second solution and is fluidly connected to the second lumen. In another embodiment, the two solutions can be mixed prior to injection, and injected as a single composition into the LAA.

Upon injection into the LAA, the fluid biomaterial increases in viscosity to form a biocompatible polymeric matrix. If desired, an accelerator (e.g., a catalyst or UV light) can be supplied to increase cure rate, initiate curing, and/or ensure thorough curing of the fluid polymeric matrix.

In some embodiments, during the procedure described above, the patient can be positioned in a posture which is effective to facilitate occlusion of the LAA. For example, the patient can be positioned at an angle relative to the ground which is effective to facilitate injection of the fluid biomaterial into the LAA of the patient. By positioning the patient at an angle (e.g., approximately a 300 to 400 angle relative to horizontal), gravity can assist the flow of the fluid biomaterial into the LAA, facilitating complete occlusion of the LAA.

In general, the methods described herein are performed percutaneously, for example using a delivery catheter assembly as discussed above. Alternatively, the fluid biomaterial and occlusion device can be introduced intraoperatively during an invasive procedure, or ancillary to another procedure which gives access to the LAA.

The methods described herein can be used to occlude the LAA, thus decreasing the risk of thromboembolic events associated with AF.

In some cases, the patient treated using the methods described herein exhibits AF. In patients with non-rheumatic AF, the risk of stroke can be estimated by calculating the patient's CHA₂DS₂-VASc score. A high CHA₂DS₂-VASc score corresponds to a greater risk of stroke, while a low CHA-DS₂-VASc score corresponds to a lower risk of stroke. In some embodiments, the patient treated using the methods described has a CHA₂DS₂-VASc score of 2 or more.

In certain embodiments, the patient is contraindicated for anticoagulation therapy. For example, the patient can have an allergy to one or more common anticoagulants (e.g. warfarin), can express a preference to not be treated with anticoagulants, can be taking another medication that interacts unfavorably with an anticoagulant, or can be at risk for hemorrhage.

### Examples

### Example 1: Synthesis and Evaluation of an Example Fluid Biomaterial

### Preparation of Hydrogel Precursor Molecules

Tetra-functional PEG-thiol (PEG4SH) (82.7% activity) was purchased from Sunbio (Anyang City, South Korea), and used for hydrogel formation without further purification or modification.

Dextran from Leuconosloc mesenteroides (average MW=15,000-20,000 Da), divinyl sulfone (DVS; 97%, MW=118.15 Da), and 3-mercaptopropionic acid (MW=106.14 Da) were purchased from Sigma-Aldrich (St. Louis, MO). The synthesis of dextran vinyl sulfone (DextranVS) containing an ethyl spacer was performed using N,N0-dicyclohexyl-carbodiimide (DCC, Fisher Scientific) and 4-(dimethylamino)pyridinium 4-toluenesulfonate (DPTS) as catalysts. DPTS was prepared using methods known in the art. Briefly, 5.0 g of p-TSA monohydrate was dissolved in 100 ml THF. 4-(Dimethylamino)-pyridine (DMAP, 99%) (Sigma-Aldrich, St. Louis, MO) at one molar equivalent top-TSA was added to this mixture. The mixture was subsequently filtered to isolate a precipitate which was further dissolved in dichloromethane (DCM, Fisher Scientific) and recrystallized using rotary vacuum evaporator.

Dextran vinyl sulfone ester synthesis was performed by adding 2.5 or 5.0 g DVS in 90 mL of inert nitrogen saturated DMSO, followed by dropwise addition of 3-MPA to it under continuous stirring. The reaction was continued for 4 hours in the dark. Dextran was dissolved in 30 mL DMSO, and a solution of DCC and p-TSA in 30 ml DMSO was added dropwise. The reaction mixture was stirred until a clear solution was obtained. Finally, the mixture was added to DVS/MPA solution in the dark, and reaction was allowed to proceed for 24 hours at room temperature.

After the completion of reaction, N,N-dicyclohexylurea (DCU) salt was filtered using a vacuum filter and the product was recovered by precipitation in 1000 mL of ice cold 100% ethanol. The precipitate was separated from residual ethanol through centrifugation at 3000 rpm for 15 min., followed by vacuum drying. The precipitate was re-dissolved in at least 100 mL of de-ionized water (pH adjusted to 7.8) and vortexed to obtain a clear solution. Finally, un-reacted polymer was removed via ultra-filtration using an Amicon filter (MWCO=10,000 Da, Millipore). The resulting viscous product was lyophilized to remove water. Vinyl sulfone substitution was confirmed and degree of substitution (DS) was determined via NMR spectroscopy.

### Formation of PEG-Dextran Hydrogels

Controlled masses of PEG and dextran vinyl sulfone were mixed with a controlled volume of TEA buffer. Two different types of dextran vinyl sulfone (DS 5 and DS10) were examined. Samples were made with varying concentrations of hydrogel in the buffer, measured in terms of wt. %/vol. Samples ranging from 10%-40% wt./vol. were evaluated. The PEG and dextran components were mixed in a 1:1 stoichiometric ratio.

### Characterization of PEG-Dextran Hydrogels

The materials properties of the PEG-Dextran hydrogels, as well as solutions of the hydrogel precursor molecules were evaluated.

### Measurement of Density and Viscosity

A controlled volume (500 μL) of each sample was collected, and weighed. Knowing the mass and volume, density was calculated. The densities measured for solutions of hydrogel precursor molecules are included in Table 1.

TABLE 1
Density measurements for solutions of hydrogel precursor molecules.
Table 1: Density (g/cm³) of Materials

Concentration

| | | 10% | 20% | 30% | 40% |
|---|---|---|---|---|---|
| Material | PEG | 1.080 | 1.202 | 1.295 | 1.393 |
| | Dextran DS 5 | 1.120 | 1.191 | 1.305 | 1.395 |
| | Dextran DS 10 | 1.107 | 1.193 | 1.306 | 1.399 |

Samples of hydrogel components were prepared as described previously, and heated to 37° C. in a water bath. Solutions of the hydrogel precursor molecules were also measured. Kinematic viscosities were measured using size 75 and size 150 Canon Manning Semi-Micro glass capillary viscometers. Once kinematic viscosity was measured, dynamic viscosity was calculated using the following relation:

$$υ = \frac{μ}{ρ}$$

Where ν is kinematic viscosity, ρ is dynamic viscosity, and ρ is density of the measured material. For each sample, viscosity was measured 12 times to ensure accuracy. The dynamic and kinematic viscosities of solutions of hydrogel precursor molecules at different concentrations are included in Table 2. The standard deviation of all measurements was found to be relatively small (<1.4% for all hydrogels measured).

TABLE 2
Dynamic and kinematic viscosities of solutions
of hydrogel precursor molecules.

| Concentration | | | |
|---|---|---|---|
| 10% | 20% | 30% | 40% |

Dynamic Viscosity (cP) of Materials

| | | | | | |
|---|---|---|---|---|---|
| Material | PEG | 2.646 | 7.773 | 24.729 | 32.810 |
| | Dextran DS 5 | 1.789 | 3.859 | 7.371 | 11.871 |
| | Dextran DS 10 | 1.607 | 4.075 | 6.478 | 16.335 |

Kinematic Viscosity (cStr) of Materials

| | | | | | |
|---|---|---|---|---|---|
| Material | PEG | 2.452 | 6.467 | 19.100 | 23.556 |
| | Dextran DS 5 | 1.598 | 3.239 | 5.649 | 8.510 |
| | Dextran DS 10 | 1.451 | 3.416 | 4.960 | 11.673 |

### Measurement of Degradation Rate

Samples of PEG and Dextran were mixed in a 1:1 ratio and allowed to solidify. In this study, 150 uL of each component was used. Samples were allowed to sit for two hours to allow complete solidification. To simulate human body conditions, samples were then submerged in a 0.01% PBS buffer (PH 7.4), and rotated in a 37° C. incubator. Samples were weighed at specified time intervals to gauge what percentage of material remained.

The results of the degradation trials are included in Table 3. After 90 days anywhere between 40-63% of hydrogel (by mass) had degraded. At 120 days, anywhere between 44-95% of hydrogel by mass had degraded.

TABLE 3
Degradation results for PEG-dextran hydrogels.

| | DS 5 | DS 10 | DS 5 | DS 10 | DS 5 | DS 10 |
|---|---|---|---|---|---|---|
| Day | 20% | 20% | 30% | 30% | 40% | 40% |

Mass/Original Mass (Swelling Ratio)

| | | | | | | |
|---|---|---|---|---|---|---|
| 0 | 1.00 | 1.00 | 1.00 | 1.00 | 1.00 | 1.00 |
| 1 | 1.52 | 1.74 | 3.13 | 3.30 | 3.32 | 3.73 |
| 2 | 2.55 | 2.49 | 3.03 | 3.37 | 3.46 | 5.89 |
| 8 | 2.63 | 2.68 | 3.43 | 3.09 | 3.70 | 4.16 |
| 15 | 2.28 | 2.52 | 2.62 | 2.85 | 2.86 | 3.41 |
| 23 | 2.02 | 2.31 | 2.15 | 2.78 | 2.90 | 3.04 |
| 29 | 2.44 | 2.07 | 2.15 | 2.66 | 2.85 | 3.44 |
| 42 | 2.04 | 1.73 | 1.88 | 2.28 | 2.32 | 2.98 |
| 54 | 2.02 | 1.71 | 1.61 | 2.17 | 2.31 | 2.61 |
| 64 | 2.01 | 2.11 | 1.87 | 2.33 | 2.62 | 2.82 |
| 79 | 1.95 | 1.95 | 1.73 | 1.95 | 2.20 | 2.44 |
| 86 | 1.66 | 1.76 | 1.57 | 2.08 | 1.99 | 2.40 |
| 92 | 1.59 | 1.74 | 1.28 | 1.79 | 1.94 | 2.29 |
| 99 | 1.52 | 1.60 | 1.28 | 1.93 | 1.88 | 2.51 |
| 106 | 1.62 | 1.59 | 1.30 | 1.96 | 2.10 | 2.48 |
| 113 | 1.51 | 1.59 | 1.26 | 1.90 | 1.95 | 2.40 |
| 120 | 1.47 | 1.71 | 1.29 | 1.79 | 1.94 | 2.43 |
| 127 | 1.46 | 1.59 | | 1.71 | 1.92 | 2.35 |

% Remaining

| | | | | | | |
|---|---|---|---|---|---|---|
| 0 | | | | | | |
| 1 | | | | | | |
| 2 | | | | 1.00 | | 1.00 |
| 8 | 1.00 | 1.00 | 1.00 | 0.92 | 1.00 | 0.71 |
| 15 | 0.87 | 0.94 | 0.76 | 0.85 | 0.77 | 0.58 |
| 23 | 0.77 | 0.86 | 0.63 | 0.83 | 0.78 | 0.52 |
| 29 | 0.93 | 0.77 | 0.63 | 0.79 | 0.77 | 0.58 |
| 42 | 0.78 | 0.64 | 0.55 | 0.68 | 0.63 | 0.51 |
| 54 | 0.77 | 0.64 | 0.47 | 0.64 | 0.62 | 0.44 |
| 64 | 0.77 | 0.79 | 0.55 | 0.69 | 0.71 | 0.48 |
| 79 | 0.74 | 0.73 | 0.50 | 0.58 | 0.59 | 0.41 |
| 86 | 0.63 | 0.66 | 0.46 | 0.62 | 0.54 | 0.41 |
| 92 | 0.61 | 0.65 | 0.37 | 0.53 | 0.52 | 0.39 |
| 99 | 0.58 | 0.60 | 0.37 | 0.57 | 0.51 | 0.43 |
| 106 | 0.62 | 0.59 | 0.38 | 0.58 | 0.57 | 0.42 |
| 113 | 0.57 | 0.59 | 0.37 | 0.56 | 0.53 | 0.41 |
| 120 | 0.56 | 0.64 | 0.38 | 0.53 | 0.52 | 0.41 |
| 127 | 0.56 | 0.59 | 0.00 | 0.51 | 0.52 | 0.40 |

% Degraded

| | | | | | | |
|---|---|---|---|---|---|---|
| 0 | | | | | | |
| 1 | | | | | | |
| 2 | | | | 0.00 | | 0.00 |
| 8 | 0.00 | 0.00 | 0.00 | 0.08 | 0.00 | 0.29 |
| 15 | 0.13 | 0.06 | 0.24 | 0.15 | 0.23 | 0.42 |
| 23 | 0.23 | 0.14 | 0.37 | 0.17 | 0.22 | 0.48 |
| 29 | 0.07 | 0.23 | 0.37 | 0.21 | 0.23 | 0.42 |
| 42 | 0.22 | 0.36 | 0.45 | 0.32 | 0.37 | 0.49 |
| 54 | 0.23 | 0.36 | 0.53 | 0.36 | 0.38 | 0.56 |
| 64 | 0.23 | 0.21 | 0.45 | 0.31 | 0.29 | 0.52 |
| 79 | 0.26 | 0.27 | 0.50 | 0.42 | 0.41 | 0.59 |
| 86 | 0.37 | 0.34 | 0.54 | 0.38 | 0.46 | 0.59 |
| 92 | 0.39 | 0.35 | 0.63 | 0.47 | 0.48 | 0.61 |
| 99 | 0.42 | 0.40 | 0.63 | 0.43 | 0.49 | 0.57 |
| 106 | 0.38 | 0.41 | 0.62 | 0.42 | 0.43 | 0.58 |
| 113 | 0.43 | 0.41 | 0.63 | 0.44 | 0.47 | 0.59 |
| 120 | 0.44 | 0.36 | 0.62 | 0.47 | 0.48 | 0.59 |
| 127 | 0.44 | 0.41 | 1.00 | 0.49 | 0.48 | 0.60 |

### Measurement of Equilibrium Swelling Ratio

Hydrogel samples were obtained and massed. The hydrogel samples were then incubated in PBS buffer (10 mM phosphate buffered saline, e.g., P3813-powder from Sigma yields a buffer of 0.01 M phosphate, 0.0027 M potassium chloride and 0.138 M sodium chloride, pH 7.4). Upon incubation, the hydrogel samples swelled, and increased mass. Every −168 hours (7 days), the sample was removed from buffer, and massed. The equilibrium swelling ratio, defined as:

$$equilibrium swelling ratio = \frac{{w}_{t}}{{w}_{o}}$$

where wₜ is the maximum swollen weight, and wo is the unswollen weight of hydrogel was determined for each hydrogel sample. Equilibrium swelling ratio was typically observe 48-72 hours after submerging samples in PBS buffer. The equilibrium swelling ratios of each hydrogel are included in Table 4 below.

TABLE 4
Equilibrium swelling ratios of PEG-Dextran hydrogels.

| Dextran | Concentration of Hydrogel Components | | |
|---|---|---|---|
| Component | 20% | 30% | 40% |
| Dextran DS 5 | 2.6 | 3.4 | 3.7 |
| Dextran DS 10 | 2.6 | 3.3 | 5.8 |

### Measurement of Cure Time

The cure time of PEG-dextran hydrogels was evaluated using a tipping vial methods. PEG and Dextran DS 5 suspensions were prepared, as described above, at different concentrations by mixing either PEG or dextran material with TEA buffer. Suspensions were mixed to a specific concentration. PEG and dextran suspensions of equal concentrations were then mixed in a 1:1 ratio in a sealed vial. The vial was shaken with an ultrasonic shaker to ensure complete mixing. Once mixing was complete, a timer was started. The vial was continually tipped or flipped. When mixed components stop moving upon actuation of the vial, the hydrogel is considered gelled, and the timer was stopped.
For each concentration, 5 solidification trials were performed. The average cure time (in seconds) for each PEG-Dextran DS 5 hydrogel measured is included in the Table 5 below, along with the standard deviation for each cure time.

TABLE 5
Cure time of of PEG-Dextran DS 5 hydrogels.

| Hydrogel | Mean Solidification Time | Standard Deviation |
|---|---|---|
| Concentration | (seconds) | (seconds) |
| 20% | 99 | 2.94 |
| 30% | 84 | 5.48 |
| 40% | 58.25 | 2.22 |

Concentration of the hydrogel sample was found to be inversely proportional to the cure time. In addition, a statistically significant correlation between concentration and cure time was observed. This was believed to be due to higher concentrations exhibiting a faster rate of crosslinking. The standard deviations for cure time were also relatively small (<6.6% of mean cure time for all samples), indicating solidification times are relatively consistent from sample to sample.

### Measurement of Volumetric Swelling Ratio

During the studies described above, maximum swelling was typically observed after 1-2 days submerged in buffer. Generally, higher concentrations and DS numbers resulted in hydrogels that exhibited greater swelling in terms of mass. To better understand the relationship between material configurations and volumetric swelling, the volumetric swelling of various hydrogel samples was evaluated.

Samples from the cure time trials described above were allowed to sit for 2 hours to solidify completely. For each aqueous solution concentration, 5 separate hydrogel samples were used. Samples were then submerged in PBS buffer, and allowed to swell. Every 24 hours, samples were removed from the buffer and dried, and the sample's volume was measured using a graduated cylinder. Volumetric swelling ratio is defined as:

$$volumetric swelling ratio = \frac{{V}_{t}}{{V}_{o}}$$

where Vₜ is the swollen volume of the hydrogel sample and Vₒ is the unswollen volume of the hydrogel sample. The mean volumetric swelling ratios of the PEG and Dextran DS 5 samples at 24 hours, 48 hours, and 72 hours are plotted in FIG. 5.

In general, aqueous solutions with higher concentrations resulted in hydrogels that exhibited higher volumetric swelling. Most of this swelling was found to occur within the first 24 hours of incubation in buffer.

### Hydrogel Anchoring Analysis

To be suitable for occlusion of the LAA, the hydrogel should remain anchored in the LAA following transport and solidification in the LAA during occlusion. The swelling of hydrogel samples over time (determined above) was used to estimate the interface pressures exerted by various hydrogel samples. The anchoring force resulting from these estimated interface pressures could then be estimated.

The degradation results described above evaluated the hydrogel composition over time. From these trials, it was possible to extrapolate a projected equilibrium swelling ratio (based on mass measurements) for hydrogel samples at 180 days (the estimated time period needed for complete endothelial tissue overgrowth over the opening of the LAA). While volumetric swelling ratios would provide a more accurate projection, long-term volumetric degradation data was not available for analysis. As a result, it was assumed that volumetric degradation was approximately proportional to mass-based degradation.

For purposes of this estimate, it was assumed that the hydrogel undergoes isotropic swelling, and that the swelling in terms of mass corresponded to a dimensionally similar volumetric swelling. Change in hydrogel plug radius can be predicted based on these swelling ratios with the following relationship:

$$Radius Multiple , {R}_{m} = \frac{{R}_{swollen}}{{R}_{initial}} = {Q}_{m}^{1 / 3}$$

Tables 6 includes the projected Qₘ and Rₘ at 180 days.

TABLE 6
Projected swelling ratio and material radius at 180 days.

| Hydrogel Sample | w/wₒ at 180 Days | R/Rₘ at 180 Days |
|---|---|---|
| PEG-Dextran DS 5 20% | 1.449 | 1.131 |
| PEG-Dextran DS 5 30% | 1.117 | 1.037 |
| PEG-Dextran DS 5 40% | 1.767 | 1.208 |
| PEG-Dextran DS 10 20% | 1.509 | 1.146 |
| PEG-Dextran DS 10 30% | 1.686 | 1.190 |
| PEG-Dextran DS 10 40% | 2.165 | 1.293 |

To analyze anchoring force, the interface pressure was then estimated. The interface pressure between the hydrogel plug and LAA tissue was defined with the following equation:

$$Interface Pressure , {p}_{i} = \frac{{δ}_{r}}{\frac{R}{{E}_{O}} ( \frac{{r}_{O}^{2} + {R}^{2}}{{r}_{O}^{2} - {R}^{2}} + {v}_{O} ) + \frac{R}{{E}_{i}} ( 1 - {v}_{i} )}$$

where δᵣ is the change in hydrogel radius due to swelling; R is the initial radius of the hydrogel plug; rₒ is the outer diameter of LAA tissue; vₒ and vᵢ are Poisson's ratio for tissue and the hydrogel, respectively, and Eₒ and Eₗ are the elastic modulus of the tissue and hydrogel, respectively. Once estimated, the interface pressure was used to calculate the anchoring force of the hydrogel using the equation below:

$$F = π {dL}_{h} {p}_{i} {μ}_{f}$$

where pᵢ is the interface pressure described previously; d is the diameter of the hydrogel (equal to 2R); Lₕ is the length of contact surface between tissue and hydrogel; and μ_{f} is the coefficient of friction between LAA tissue and the hydrogel. The values used for the variables in the anchoring force analysis are included in Table 7 below.

TABLE 7
Values used for the variables in the anchoring force analysis.

| Variable | Description | Value | |
|---|---|---|---|
| δᵣ | Change in hydrogel radius | Varies by sample | |
| R | Initial radius of hydrogel | 10 | mm |
| r | Initial radius of hydrogel and tissue | 11 | mm |
| Eₒ | Elastic modulus of tissue | 40.8 | kPa |
| E₁ | Elastic modulus of hydrogel | 14 | kPa |
| νₒ | Poisson Ratio of tissue | 0.45 | |
| ν₁ | Poisson Ratio of hydrogel | 0.5 | |
| d | Diameter of hydrogel | 20 | mm |
| Lₕ | Length of hydrogel | 30 | mm |

| | | |
|---|---|---|
| μ_{f} | Coefficient of friction | 0.332 |

The anchoring force for each hydrogel sample was compared to the estimated weight of the hydrogel plug. Samples were determined to exhibit adequate anchoring force for use in occlusion of the LAA if the hydrogel plug exhibited an estimated anchoring force at least as large as the weight of the hydrogel plug. Experimentally measured values for density were used to estimate the weight of each hydrogel plug. Table 8 below shows the estimated weight and anchoring force of each hydrogel plug, based upon projected swelling ratios at day 180 and the equations above.

TABLE 8
Estimated weight and anchoring force for plugs
formed from various PEG-Dextran hydrogels.

| | Hydrogel | Plug | Anchoring | Anchoring |
|---|---|---|---|---|
| | Density | Weight | Force | Force/Plug |
| Hydrogel Sample | (g/cm³) | (N) | (N) | Weight |
| PEG-Dextran DS 5 20% | 1.195 | 0.221 | 0.277 | 1.253 |
| PEG-Dextran DS 5 30% | 1.300 | 0.240 | 0.078 | 0.325 |
| PEG-Dextran DS 5 40% | 1.395 | 0.258 | 0.444 | 1.721 |
| PEG-Dextran DS 10 20% | 1.195 | 0.221 | 0.310 | 1.403 |
| PEG-Dextran DS 10 30% | 1.300 | 0.240 | 0.403 | 1.679 |
| PEG-Dextran DS 10 40% | 1.395 | 0.258 | 0.631 | 2.445 |

Based on the results above, all samples other than the DS 5 30%, exhibited estimated anchoring forces at least as large as the weight of the hydrogel plug with a safety factor of 1.25 or higher. The low value for the DS 5 30% sample was likely due to anomalous degradation data. A more reasonable range for anchoring force would be 0.277-0.444N (in between DS 5 20% and DS 5 40% samples), which would suggest that the DS 5 30% material would also exhibit adequate anchoring force for use in occlusion of the LAA.

It is worth noting that this analysis represents the most conservative anchoring scenario. Additional anchoring forces due to blood pressure and geometric anomalies (e.g., irregular trabeculae or multiple lobes in the LAA) were not considered. Blood pressure would likely only act on the outer surface of the hydrogel (the surface facing the LA), providing additional anchoring force. Irregular trabeculae and geometric asymmetries in the LAA would also cause additional anchoring, but these were not quantified due to unpredictable geometry in the LAA. Both of these would add additional anchoring, and provide further evidence that this hydrogel is suitable for application in occluding the LAA.

The devices and methods of the appended claims are not limited in scope by the specific devices, systems, kits, and methods described herein, which are intended as illustrations of a few aspects of the claims. Any devices, systems, and methods that are functionally equivalent are intended to fall within the scope of the claims. Various modifications of the devices, systems, kits, and methods in addition to those shown and described herein are intended to fall within the scope of the appended claims. Further, while only certain representative devices, systems, kits, and method steps disclosed herein are specifically described, other combinations of the devices, systems, kits, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A method of occluding the left atrial appendage (LAA) of a patient, wherein the LAA extends from a left atrium of the patient's heart and has an internal volume and an ostium at its juncture with the left atrium, the method comprising:
positioning an occlusion device within the ostium of the LAA,
wherein the occlusion device comprises an occluder portion comprising a proximal end and a distal end, the proximal end coupled to a hub having an injection lumen passing axially therethrough; and an anchor portion operably coupled to the occluder portion; and
wherein when the occlusion device is positioned within the ostium of the LAA, the anchor portion extends into the internal volume of the LAA;
injecting a fluid biomaterial into the LAA of the patient through the injection lumen, wherein the fluid biomaterial exhibits a cure time following injection during which it remains flowable but after which it solidifies to form a biocompatible polymeric matrix that fills and occupies the internal volume of the LAA; and
retaining the occlusion device within the ostium of the LAA until the cure time has elapsed and the fluid biomaterial has solidified to form the biocompatible polymeric matrix.

2. The method of claim 1, wherein the occluder portion is configured to move between an occluder-deployed state and an occluder-nondeployed state, and
wherein the anchor portion is configured to move between an anchor-deployed state and an anchor-nondeployed state.

3. The method of any of claims 1-2, wherein the anchor portion comprises a plurality of anchor segments, wherein each of the plurality of anchor segments extend distally beyond the occluder portion when the occlude portion is in the occlude-deployed state and the anchor portion is in the anchor-deployed state.

4. The method of claim 3, wherein each of the anchor segments comprises a loop portion, a helical portion, a fin portion, a barb portion, or any combination thereof.

5. The method of any of claims 1-4, wherein the anchor portion is coupled to the occluder portion by way of the hub.

6. The method of any of claim 1-5, wherein the occluder portion comprises a tissue growth member extending between the proximal end and the distal end of the occluder portion.

7. The method of claim 6, wherein the tissue growth member comprises a layer formed from an expanded polytetrafluoroethylene (ePTFE).

8. The method of any of claims 1-7, wherein the hub further comprises a second lumen passing axially therethrough, wherein the second lumen is fluidly isolated from the injection lumen.

9. The method of claim 8, wherein the injection channel terminates distally at an injection outlet and the second lumen terminates distally at a fluid inlet.

10. The method of claim 9, wherein the injection outlet is separated from and distal to the fluid inlet.

11. The method of any of claims 1-10, wherein the occlusion device is positioned within the ostium of the LAA using a delivery system, wherein the delivery system comprises:
a delivery catheter, wherein the delivery catheter comprises:
a delivery catheter body extending between a proximal end and a distal end, the catheter body comprising a wall structure that defines at least one injection channel extending from the proximal end to the distal end and terminating in an outlet opening;
a handle coupled to the proximal end of the delivery catheter body, and
the occlusion device operatively coupled to the handle and coupled to the distal end of the delivery catheter body,
wherein the outlet opening of the at least one injection channel is fluidly connected to the injection lumen of the occlusion device.

12. The method of claim 11, wherein the delivery system further comprises a sheath having a proximal end portion, a distal end portion having a distal tip, and a wall circumferentially enclosing a sheath lumen extending along an entire length of the sheath;
wherein the delivery catheter is sized to be received and selectively advanceable within the sheath lumen such that the occlusion device can be passed through the sheath lumen to a position distal of the distal tip.

13. The method of claim 12, wherein the sheath further comprises:
at least one inflation channel within the wall of the sheath; and
a balloon coupled to the distal end portion of the sheath and positioned in fluid communication with the at least one inflation channel of the sheath, the balloon enclosing an interior space.

14. The method of claim 13, wherein the wall of the delivery catheter body defines at least one outlet opening to provide fluid communication between the at least one inflation channel and the interior space of the balloon.

15. The method of any of claims 11-14, wherein the at least one injection channel of the delivery catheter body comprises a plurality of injection channels.

16. The method of any of claims 11-15, wherein the at least one injection channel of the delivery catheter body comprises first and second injection channels, and wherein the wall structure of the delivery catheter body comprises an outer wall and an inner wall that extends between opposing portions of the outer wall to define the first and second injection channels.

17. The method of claim 16, further comprising a mixing component positioned between first and second injection channels and the outlet opening.

18. The method of any of claims 11-17, wherein the delivery catheter further comprises an auxiliary lumen fluidly isolated from the at least one injection channel and extending from the proximal end to the distal end and terminating in an auxiliary opening, and
wherein the auxiliary opening of the auxiliary lumen is fluidly connected to the second lumen of the occlusion device.

19. A method of occluding the LAA of a patient comprising
(a) advancing a delivery system percutaneously through the patient's vasculature to reach the patient's right atrium, the delivery system comprising:
a delivery catheter, wherein the delivery catheter comprises:
a delivery catheter body extending between a proximal end and a distal end, the catheter body comprising a wall structure that defines at least one injection channel extending from the proximal end to the distal end and terminating in an outlet opening;
a handle coupled to the proximal end of the delivery catheter body, and
an occlusion device operatively coupled to the handle and coupled to the distal end of the delivery catheter body, wherein the occlusion device comprises an occluder portion comprising a proximal end and a distal end, the proximal end coupled to a hub having an injection lumen passing axially therethrough; and an anchor portion operably coupled to the occluder portion;
wherein the outlet opening of the at least one injection channel is fluidly connected to the injection lumen of the occlusion device
(b) advancing the delivery system through an opening in the interatrial septum to reach the patient's left atrium;
(c) deploying the occlusion device within the ostium of the LAA such that the anchor portion extends into the internal volume of the LAA;
(d) injecting a fluid biomaterial into the LAA through the injection channel of the delivery catheter body and the injection lumen of the occlusion device, wherein the fluid biomaterial exhibits a cure time following injection during which it remains flowable but after which it solidifies to form a biocompatible polymeric matrix that fills and occupies the internal volume of the LAA;
(e) retaining the occlusion device within the ostium of the LAA until the cure time has elapsed and the fluid biomaterial has solidified to form the biocompatible polymeric matrix.

20. The method of claim 19, further comprising forming an opening in the interatrial septum of the patient's heart.

21. The method of any of claims 19-20, wherein the occluder portion is configured to move between an occluder-deployed state and an occluder-nondeployed state, and
wherein the anchor portion is configured to move between an anchor-deployed state and an anchor-nondeployed state.

22. The method of any of claims 19-21, wherein the anchor portion comprises a plurality of anchor segments, wherein each of the plurality of anchor segments extend distally beyond the occluder portion when the occlude portion is in the occlude-deployed state and the anchor portion is in the anchor-deployed state.

23. The method of claim 22, wherein each of the anchor segments comprises a loop portion, a helical portion, a fin portion, a barb portion, or any combination thereof.

24. The method of any of claims 19-23, wherein the anchor portion is coupled to the occluder portion by way of the hub.

25. The method of any of claim 19-24, wherein the occluder portion comprises a tissue growth member extending between the proximal end and the distal end of the occluder portion.

26. The method of claim 25, wherein the tissue growth member comprises a layer formed from an expanded polytetrafluoroethylene (ePTFE).

27. The method of any of claims 19-26, wherein the hub further comprises a second lumen passing axially therethrough, wherein the second lumen is fluidly isolated from the injection lumen.

28. The method of claim 27, wherein the injection channel terminates distally at an injection outlet and the second lumen terminates distally at a fluid inlet.

29. The method of claim 28, wherein the injection outlet is separated from and distal to the fluid inlet.

30. The method of any of claims 27-29, wherein the delivery catheter further comprises an auxiliary lumen fluidly isolated from the at least one injection channel and extending from the proximal end to the distal end and terminating in an auxiliary opening, and
wherein the auxiliary opening of the auxiliary lumen is fluidly connected to the second lumen of the occlusion device.

31. The method of any of claims 27-30, wherein when the fluid biomaterial is injected into the LAA through the injection channel of the delivery catheter body and the injection lumen of the occlusion device, blood present in the LAA flows from the LAA into the second lumen.

32. The method of any of claims 19-31, wherein the delivery system further comprises a sheath having a proximal end portion, a distal end portion having a distal tip, and a wall circumferentially enclosing a sheath lumen extending along an entire length of the sheath;
wherein the delivery catheter is sized to be received and selectively advanceable within the sheath lumen such that the occlusion device can be passed through the sheath lumen to a position distal of the distal tip.

33. The method of claim 32, wherein the sheath further comprises:
at least one inflation channel within the wall of the sheath; and
a balloon coupled to the distal end portion of the sheath and positioned in fluid communication with the at least one inflation channel of the sheath, the balloon enclosing an interior space.

34. The method of claim 33, wherein the method further comprises inflating the balloon to anchor and secure the delivery system within the left atrium.

35. The method of any of claims 19-34, further comprising estimating the internal volume of the LAA to determine the amount of fluid biomaterial injected to fill and occupy the internal volume of the patient's LAA.

36. The method of claim 35, wherein estimating the internal volume of the LAA comprised removing the blood present in the LAA prior to injecting the fluid biomaterial into the LAA of the patient and measuring the volume of blood removed from the LAA.

37. The method of claim 35, wherein estimating the internal volume of the LAA comprises imaging the LAA.

38. The method of any of claims 19-37, further comprising withdrawing blood from the LAA prior to injection of the fluid biomaterial.

39. The method of any of claims 19-38, wherein the advancement of the delivery catheter assembly is monitored by radiographic imaging, sonographic imaging, or combinations thereof.

40. The method of any of claims 19-39, wherein step (a) comprises advancing the delivery catheter through the femoral vein.

41. A method of occluding the left atrial appendage (LAA) of a patient, wherein the LAA extends from a left atrium of the patient's heart and has an internal volume and an ostium at its juncture with the left atrium, the method comprising:
injecting a fluid biomaterial into the LAA of the patient, wherein the fluid biomaterial exhibits a cure time following injection during which it remains flowable but after which it solidifies to form a biocompatible polymeric matrix that fills and occupies the internal volume of the LAA;
positioning an occlusion device within the ostium of the LAA during the cure time,
wherein the occlusion device comprises an occluder portion and an anchor portion operably coupled to the occluder portion; and
wherein when the occlusion device is positioned within the ostium of the LAA, the anchor portion extends into the internal volume of the LAA; and
retaining the occlusion device within the ostium of the LAA until the cure time has elapsed and the fluid biomaterial has solidified to form the biocompatible polymeric matrix.

42. The method of claim 41, wherein the occlusion device further comprises a hub,
wherein the occluder portion comprises a proximal end and a distal end, the proximal end coupled to the hub,
wherein the occluder portion is configured to move between an occluder-deployed state and an occluder-nondeployed state, and
wherein the anchor portion is configured to be moved between an anchor-deployed state and an anchor-nondeployed state.

43. The method of any of claims 41-42, wherein the anchor portion comprises a plurality of anchor segments, wherein each of the plurality of anchor segments extend distally beyond the occluder portion when the occlude portion is in the occlude-deployed state and the anchor portion is in the anchor-deployed state.

44. The method of claim 43, wherein each of the anchor segments comprises a loop portion, a helical portion, a fin portion, a barb portion, or any combination thereof.

45. The method of any of claims 41-44, wherein the anchor portion is coupled to the occluder portion by way of the hub.

46. The method of any of claim 41-45, wherein the occluder portion comprises a tissue growth member extending between the proximal end and the distal end of the occluder portion.

47. The method of claim 46, wherein the tissue growth member comprises a layer formed from an expanded polytetrafluoroethylene (ePTFE).

48. The method of any of claims 41-47, wherein the fluid biomaterial is injected into the LAA of the patient using a delivery catheter assembly,
wherein the delivery catheter assembly comprises:
a first catheter body having a proximal end portion, a distal end portion having a distal tip, and a wall that circumferentially encloses a primary opening, wherein the first catheter body further comprises at least one inflation channel within the wall of the first catheter body, wherein the primary opening of the first catheter body extends along an entire length of the first catheter body;
a first balloon coupled to the distal end portion of the first catheter body and positioned in fluid communication with the at least one inflation channel of the first catheter body, the first balloon enclosing an interior space, wherein the first catheter body extends through the interior space of the first balloon in a proximal-to-distal direction such that at least the distal tip of the first catheter body is positioned distal of the first balloon;
a second catheter body partially received within the primary opening of, and selectively moveable relative to, the first catheter body, wherein the second catheter body has a proximal end portion, a distal end portion having a tip, and a wall that circumferentially encloses a primary opening, wherein the second catheter body further comprises at least one inflation channel within the wall of the second catheter body, wherein the primary opening of the second catheter body extends along an entire length of the second catheter body;
a second balloon coupled to the distal end portion of the second catheter body and positioned in fluid communication with the at least one inflation channel of the second catheter body, the second balloon enclosing an interior space, wherein the second catheter body extends through the interior space of the second balloon in the proximal-to-distal direction such that at least the distal tip of the second catheter body is positioned distal of the second balloon; and
a third catheter body partially received within the primary opening of, and selectively moveable relative to, the second catheter body, wherein the third catheter body has a proximal end portion, a distal end portion, and a wall structure that defines at least one injection channel extending from the proximal end portion toward the distal end portion, wherein the distal end portion of the third catheter body further comprises at least one outlet opening positioned in fluid communication with the at least one injection channel.

49. The method of claim 48, wherein the at least one injection channel of the third catheter body comprises a plurality of injection channels.

50. The method of any of claims 48-49, wherein the at least one injection channel of the third catheter body comprises first and second injection channels, and wherein the wall structure of the third catheter body comprises an outer wall and an inner wall that extends between opposing portions of the outer wall to define the first and second injection channels.

51. The method of any of claims 48-50, wherein the at least one outlet opening of the distal end portion of the third catheter body comprises a plurality of outlet openings.

52. The method of any of claims 48-51, wherein the distal end portion of the third catheter body further comprises a static mixing component positioned between the at least one injection channel and the at least one outlet opening.

53. The method of any of claims 48-52, wherein, in an inflated position, the second balloon is larger than the first balloon.

54. The method of any of claims 48-53, wherein the wall of the first catheter body defines at least one outlet opening to provide fluid communication between the at least one inflation channel and the interior space of the first balloon.

55. The method of any of claims 48-54, wherein the wall of the second catheter body defines at least one outlet opening to provide fluid communication between the at least one inflation channel and the interior space of the second balloon.

56. The method of any of claims 48-55, wherein the distal end portion of the third catheter body has a distal tip and a diaphragm that is secured to the distal tip, the diaphragm extending outwardly from the distal tip.

57. The method of any of claims 48-56, wherein the third catheter body is selectively retractable relative to the second catheter body, and wherein when the third catheter body is retracted to be fully received within the primary opening of the second catheter body, the diaphragm of the third catheter body occludes the primary opening of the second catheter body to prevent entry of material into the primary opening of the second catheter body in a distal-to-proximal direction.

58. The method of any of claims 48-57, wherein the second catheter body is selectively retractable relative to the first catheter body.

59. The method of any of claims 48-58, wherein the first and second catheter bodies are selectively lockable to maintain a desired position and orientation of the second catheter body relative to the first catheter body.

60. The method of any of claims 48-58, wherein the third catheter body is removable from the primary opening of the second catheter.

61. The method of any of claims 41-60, wherein the occlusion device is positioned within the ostium of the LAA using a delivery system,
wherein the delivery system comprises:
a delivery catheter body extending between a proximal end and a distal end, a handle coupled to the proximal end of the delivery catheter body, and
the occlusion device operatively coupled to the handle and coupled to the distal end of the delivery catheter body,

62. The method of claims 48-61, wherein the delivery system is sized to be received within the primary opening of, and selectively moveable relative to, the second catheter body such that the occlusion device can be passed through the primary opening of the second catheter body to a position distal of the second balloon.

63. A method of occluding the LAA of a patient comprising
(a) advancing a delivery catheter assembly percutaneously through the patient's vasculature to reach the patient's right atrium, the delivery catheter assembly comprising:
a first catheter body having a proximal end portion, a distal end portion having a distal tip, and a wall that circumferentially encloses a primary opening, wherein the first catheter body further comprises at least one inflation channel within the wall of the first catheter body, wherein the primary opening of the first catheter body extends along an entire length of the first catheter body;
a first balloon coupled to the distal end portion of the first catheter body and positioned in fluid communication with the at least one inflation channel of the first catheter body, the first balloon enclosing an interior space, wherein the first catheter body extends through the interior space of the first balloon in a proximal-to-distal direction such that at least the distal tip of the first catheter body is positioned distal of the first balloon;
a second catheter body partially received within the primary opening of, and selectively moveable relative to, the first catheter body, wherein the second catheter body has a proximal end portion, a distal end portion having a tip, and a wall that circumferentially encloses a primary opening, wherein the second catheter body further comprises at least one inflation channel within the wall of the second catheter body, wherein the primary opening of the second catheter body extends along an entire length of the second catheter body;
a second balloon coupled to the distal end portion of the second catheter body and positioned in fluid communication with the at least one inflation channel of the second catheter body, the second balloon enclosing an interior space, wherein the second catheter body extends through the interior space of the second balloon in the proximal-to-distal direction such that at least the distal tip of the second catheter body is positioned distal of the second balloon; and
a third catheter body partially received within the primary opening of, and selectively moveable relative to, the second catheter body, wherein the third catheter body has a proximal end portion, a distal end portion, and a wall structure that defines at least one injection channel extending from the proximal end portion toward the distal end portion, wherein the distal end portion of the third catheter body further comprises at least one outlet opening positioned in fluid communication with the at least one injection channel;
(b) advancing the delivery catheter assembly through an opening in the interatrial septum to reach the patient's left atrium;
(c) selectively inflating the first balloon to anchor and secure the delivery catheter assembly within the left atrium;
(d) selectively advancing the second catheter body relative to the first catheter body;
(e) selectively inflating the second balloon to occlude an ostium of the LAA of the patient;
(f) selectively advancing the third catheter body relative to the second catheter body;
(g) injecting a fluid biomaterial into the LAA through the injection channel of the third catheter body, wherein the fluid biomaterial exhibits a cure time following injection during which it remains flowable but after which it solidifies to form a biocompatible polymeric matrix that fills and occupies the internal volume of the LAA;
(h) removing the third catheter body from the primary opening of the second catheter;
(i) inserting a delivery system sized to be received within the primary opening of, and selectively moveable relative to, the second catheter body into the primary opening of the second catheter body, wherein the delivery system comprises:
a delivery catheter body extending between a proximal end and a distal end, a handle coupled to the proximal end of the delivery catheter body, and
the occlusion device operatively coupled to the handle and coupled to the distal end of the delivery catheter body, wherein the occlusion device comprises an occluder portion and an anchor portion operably coupled to the occluder portion;
(j) advancing the delivery system within the primary opening of the second catheter body such that the occlusion device is passed through the primary opening of the second catheter body to a position distal of the second balloon,
(k) deploying the occlusion device within the ostium of the LAA such that the anchor portion extends into the internal volume of the LAA;
(l) retaining the occlusion device within the ostium of the LAA until the cure time has elapsed and the fluid biomaterial has solidified to form the biocompatible polymeric matrix.

64. The method of claim 63, further comprising forming an opening in the interatrial septum of the patient's heart.

65. The method of any of claims 63-64, wherein the occluder portion is configured to move between an occluder-deployed state and an occluder-nondeployed state, and
wherein the anchor portion is configured to move between an anchor-deployed state and an anchor-nondeployed state.

66. The method of any of claims 63-64, wherein the anchor portion comprises a plurality of anchor segments, wherein each of the plurality of anchor segments extend distally beyond the occluder portion when the occlude portion is in the occlude-deployed state and the anchor portion is in the anchor-deployed state.

67. The method of claim 66, wherein each of the anchor segments comprises a loop portion, a helical portion, a fin portion, a barb portion, or any combination thereof.

68. The method of any of claims 63-67, wherein the anchor portion is coupled to the occluder portion by way of the hub.

69. The method of any of claim 63-68, wherein the occluder portion comprises a tissue growth member extending between the proximal end and the distal end of the occluder portion.

70. The method of claim 69, wherein the tissue growth member comprises a layer formed from an expanded polytetrafluoroethylene (ePTFE).

71. The method of any of claims 63-70, further comprising estimating the internal volume of the LAA to determine the amount of fluid biomaterial injected to fill and occupy the internal volume of the patient's LAA.

72. The method of claim 71, wherein estimating the internal volume of the LAA comprised removing the blood present in the LAA prior to injecting the fluid biomaterial into the LAA of the patient and measuring the volume of blood removed from the LAA.

73. The method of claim 71, wherein estimating the internal volume of the LAA comprises imaging the LAA.

74. The method of any of claims 63-73, further comprising withdrawing blood from the LAA prior to injection of the fluid biomaterial.

75. The method of any of claims 63-74, wherein the advancement of the delivery catheter assembly is monitored by radiographic imaging, sonographic imaging, or combinations thereof.

76. The method of any of claims 63-75, wherein step (a) comprises advancing the delivery catheter through the femoral vein.

77. A method of occluding the left atrial appendage (LAA) of a patient, wherein the LAA extends from a left atrium of the patient's heart and has an internal volume and an ostium at its juncture with the left atrium, the method comprising:
positioning an occlusion device within the ostium of the LAA,
wherein the occlusion device comprises an occluder portion comprising a proximal end and a distal end, the proximal end coupled to a hub having an injection lumen passing axially therethrough; and
injecting a fluid biomaterial into the LAA of the patient through the injection lumen, wherein the fluid biomaterial exhibits a cure time following injection during which it remains flowable but after which it solidifies to form a biocompatible polymeric matrix that fills and occupies the internal volume of the LAA;
advancing an anchoring portion through the injection lumen and coupling the anchoring portion to the occlusion device, wherein when the anchoring portion is coupled to the occlusion device, the anchor portion extends into the internal volume of the LAA; and
retaining the occlusion device within the ostium of the LAA until the cure time has elapsed and the fluid biomaterial has solidified to form the biocompatible polymeric matrix.

78. The method of claim 77, wherein the occluder portion is configured to move between an occluder-deployed state and an occluder-nondeployed state, and
wherein the anchor portion is configured to move between an anchor-deployed state and an anchor-nondeployed state.

79. The method of any of claims 77-78, wherein the anchor portion comprises a plurality of anchor segments, wherein each of the plurality of anchor segments extend distally beyond the occluder portion when the occlude portion is in the occlude-deployed state and the anchor portion is in the anchor-deployed state and coupled to the occlusion device.

80. The method of claim 79, wherein each of the anchor segments comprises a loop portion, a helical portion, a fin portion, a barb portion, or any combination thereof.

81. The method of any of claims 77-80, wherein the anchor portion is coupled to the occluder portion by way of the hub, such as by screwing the anchor portion to the hub.

82. The method of any of claim 77-80, wherein the occluder portion comprises a tissue growth member extending between the proximal end and the distal end of the occluder portion.

83. The method of claim 82, wherein the tissue growth member comprises a layer formed from an expanded polytetrafluoroethylene (ePTFE).

84. The method of any of claims 77-83, wherein the hub further comprises a second lumen passing axially therethrough, wherein the second lumen is fluidly isolated from the injection lumen.

85. The method of claim 84, wherein the injection channel terminates distally at an injection outlet and the second lumen terminates distally at a fluid inlet.

86. The method of claim 85, wherein the injection outlet is separated from and distal to the fluid inlet.

87. The method of any of claims 77-86, wherein the occlusion device is positioned within the ostium of the LAA using a delivery system, wherein the delivery system comprises:
a delivery catheter, wherein the delivery catheter comprises:
a delivery catheter body extending between a proximal end and a distal end, the catheter body comprising a wall structure that defines at least one injection channel extending from the proximal end to the distal end and terminating in an outlet opening;
a handle coupled to the proximal end of the delivery catheter body, and
the occlusion device operatively coupled to the handle and coupled to the distal end of the delivery catheter body,
wherein the outlet opening of the at least one injection channel is fluidly connected to the injection lumen of the occlusion device.

88. The method of claim 87, wherein the delivery system further comprises a sheath having a proximal end portion, a distal end portion having a distal tip, and a wall circumferentially enclosing a sheath lumen extending along an entire length of the sheath,
wherein the delivery catheter is sized to be received and selectively advanceable within the sheath lumen such that the occlusion device can be passed through the sheath lumen to a position distal of the distal tip.

89. The method of claim 88, wherein the sheath further comprises:
at least one inflation channel within the wall of the sheath; and
a balloon coupled to the distal end portion of the sheath and positioned in fluid communication with the at least one inflation channel of the sheath, the balloon enclosing an interior space.

90. The method of claim 89, wherein the wall of the delivery catheter body defines at least one outlet opening to provide fluid communication between the at least one inflation channel and the interior space of the balloon.

91. The method of any of claims 87-90, wherein the delivery catheter further comprises an auxiliary lumen fluidly isolated from the at least one injection channel and extending from the proximal end to the distal end and terminating in an auxiliary opening, and
wherein the auxiliary opening of the auxiliary lumen is fluidly connected to the second lumen of the occlusion device.

92. The method of any of claims 87-91, wherein advancing an anchoring portion through the injection lumen and coupling the anchoring portion to the occlusion device comprises advancing the anchoring portion through the at least one injection channel and through the injection lumen and coupling the anchor portion to the occlude portion.

93. The method of claim 92, wherein the anchor portion is coupled to the occluder portion by way of the hub, and wherein coupling the anchor portion comprises screwing the anchor portion to the hub.

94. A method of occluding the LAA of a patient comprising
(a) advancing a delivery system percutaneously through the patient's vasculature to reach the patient's right atrium, the delivery system comprising:
a delivery catheter, wherein the delivery catheter comprises:
a delivery catheter body extending between a proximal end and a distal end, the catheter body comprising a wall structure that defines at least one injection channel extending from the proximal end to the distal end and terminating in an outlet opening;
a handle coupled to the proximal end of the delivery catheter body, and
an occlusion device operatively coupled to the handle and coupled to the distal end of the delivery catheter body, wherein the occlusion device comprises an occluder portion comprising a proximal end and a distal end, the proximal end coupled to a hub having an injection lumen passing axially therethrough;
wherein the outlet opening of the at least one injection channel is fluidly connected to the injection lumen of the occlusion device
(b) advancing the delivery system through an opening in the interatrial septum to reach the patient's left atrium;
(c) deploying the occlusion device within the ostium of the LAA;
(d) injecting a fluid biomaterial into the LAA through the injection channel of the delivery catheter body and the injection lumen of the occlusion device, wherein the fluid biomaterial exhibits a cure time following injection during which it remains flowable but after which it solidifies to form a biocompatible polymeric matrix that fills and occupies the internal volume of the LAA;
(e) advancing an anchoring portion through the injection channel of the delivery catheter body and the injection lumen and coupling the anchoring portion to the occlusion device, wherein when the anchoring portion is coupled to the occlusion device, the anchor portion extends into the internal volume of the LAA; and
(f) retaining the occlusion device within the ostium of the LAA until the cure time has elapsed and the fluid biomaterial has solidified to form the biocompatible polymeric matrix.

95. The method of claim 94, further comprising forming an opening in the interatrial septum of the patient's heart.

96. The method of any of claims 94-95, wherein the occluder portion is configured to move between an occluder-deployed state and an occluder-nondeployed state, and
wherein the anchor portion is configured to move between an anchor-deployed state and an anchor-nondeployed state.

97. The method of any of claims 94-96, wherein the anchor portion comprises a plurality of anchor segments, wherein each of the plurality of anchor segments extend distally beyond the occluder portion when the occlude portion is in the occlude-deployed state and the anchor portion is in the anchor-deployed state and coupled to the occlusion device.

98. The method of claim 97, wherein each of the anchor segments comprises a loop portion, a helical portion, a fin portion, a barb portion, or any combination thereof.

99. The method of any of claims 94-98, wherein the anchor portion is coupled to the occluder portion by way of the hub, and wherein coupling the anchor portion comprises screwing the anchor portion to the hub.

100. The method of any of claim 94-99, wherein the occluder portion comprises a tissue growth member extending between the proximal end and the distal end of the occluder portion.

101. The method of claim 100, wherein the tissue growth member comprises a layer formed from an expanded polytetrafluoroethylene (ePTFE).

102. The method of any of claims 94-101, wherein the hub further comprises a second lumen passing axially therethrough, wherein the second lumen is fluidly isolated from the injection lumen.

103. The method of claim 102, wherein the injection channel terminates distally at an injection outlet and the second lumen terminates distally at a fluid inlet.

104. The method of claim 103, wherein the injection outlet is separated from and distal to the fluid inlet.

105. The method of any of claims 102-104, wherein the delivery catheter further comprises an auxiliary lumen fluidly isolated from the at least one injection channel and extending from the proximal end to the distal end and terminating in an auxiliary opening, and
wherein the auxiliary opening of the auxiliary lumen is fluidly connected to the second lumen of the occlusion device.

106. The method of any of claims 102-105, wherein when the fluid biomaterial is injected into the LAA through the injection channel of the delivery catheter body and the injection lumen of the occlusion device, blood present in the LAA flows from the LAA into the second lumen.

107. The method of any of claims 94-106, wherein the delivery system further comprises a sheath having a proximal end portion, a distal end portion having a distal tip, and a wall circumferentially enclosing a sheath lumen extending along an entire length of the sheath;
wherein the delivery catheter is sized to be received and selectively advanceable within the sheath lumen such that the occlusion device can be passed through the sheath lumen to a position distal of the distal tip.

108. The method of claim 107, wherein the sheath further comprises:
at least one inflation channel within the wall of the sheath; and
a balloon coupled to the distal end portion of the sheath and positioned in fluid communication with the at least one inflation channel of the sheath, the balloon enclosing an interior space.

109. The method of claim 108, wherein the method further comprises inflating the balloon to anchor and secure the delivery system within the left atrium.

110. The method of any of claims 94-109, further comprising estimating the internal volume of the LAA to determine the amount of fluid biomaterial injected to fill and occupy the internal volume of the patient's LAA.

111. The method of claim 110, wherein estimating the internal volume of the LAA comprised removing the blood present in the LAA prior to injecting the fluid biomaterial into the LAA of the patient and measuring the volume of blood removed from the LAA.

112. The method of claim 110, wherein estimating the internal volume of the LAA comprises imaging the LAA.

113. The method of any of claims 94-112, further comprising withdrawing blood from the LAA prior to injection of the fluid biomaterial.

114. The method of any of claims 94-113, wherein the advancement of the delivery catheter assembly is monitored by radiographic imaging, sonographic imaging, or combinations thereof.

115. The method of any of claims 94-114, wherein step (a) comprises advancing the delivery catheter through the femoral vein.

116. The method of any of claims 1-115, wherein the fluid biomaterial comprises a stimuli-responsive biomaterial.

117. The method of claim 116, wherein the stimuli-responsive biocompatible polymer comprises an ionically responsive complex coacervate.

118. The method of claim 117, wherein the ionically responsive complex coacervate comprises at least one polycation, at least one polyanion, and a monovalent salt,
wherein the concentration of the monovalent salt in the ionically responsive complex coacervate is greater than the concentration of the monovalent salt in the LAA.

119. The method of claim 118, wherein the concentration of the monovalent salt in the complex coacervate is 1.5 to 10 times greater than the concentration of the monovalent salt in the LAA.

120. The method of any of claims 118-119, wherein the monovalent salt in the complex coacervate is NaCl, KCl, or a mixture thereof.

121. The method of any of claims 118-120, wherein the total positive/negative charge ratio of the polycation solution to the polyanion is from 4 to 0.25 and the concentration of the monovalent salt in the complex coacervate is from 0.5 M to 2.0 M.

122. The method of any of claims 118-121, wherein the complex coacervate has a pH of 6 to 9, such as a pH of 7 to 7.5.

123. The method of any of claims 118-122, wherein the polycation comprises a polyamine with two or more amine groups.

124. The method of any of claim 123, wherein the polyamine comprises a polysaccharide, a protein, a recombinant protein, or a synthetic polyamine.

125. The method of any of claims 123-124, wherein the polyamine comprises an amine-modified natural polymer.

126. The method of any of claims 123-125, wherein the polyamine comprises gelatin modified with an alkyldiamino compound.

127. The method of any of claims 123-126, wherein the polycation comprises a polyacrylate comprising two or more pendant amino groups.

128. The method of claim 127, wherein the amino groups comprise an alkylamino group, a heteroaryl group, a guanidinyl group, an imidazole, an aromatic group substituted with one or more amino groups, a primary amino group, a secondary amino group, tertiary amino group, a quaternary amine, or any combination thereof.

129. The method of any of claims 118-128, wherein the polycation comprises a polycationic micelle or liposome.

130. The method of any of claims 118-129, wherein the polycation comprises a dendrimer having 3 to 20 arms, wherein each arm comprises a terminal amino group.

131. The method of any of claims 118-130, wherein the polycation comprises a protamine.

132. The method of any of claims 118-131, wherein the polycation comprises salmine or clupein.

133. The method of any of claims 118-132, wherein the polycation comprises a natural polymer or a synthetic polymer containing two or more guanidinyl sidechains.

134. The method of any of claims 118-133, wherein the polycation comprises a synthetic polyguanidinyl polymer comprising an acrylate or methacrylate backbone and two or more guanidinyl sidechains.

135. The method of any of claims 118-134, wherein the polycation comprises a synthetic polyguanidinyl polymer comprising a copolymer derived from a monomer selected from the group consisting of an acrylate, a methacrylate, an acrylamide, a methacrylamide, or any combination thereof and a monomer defined by Formula I below 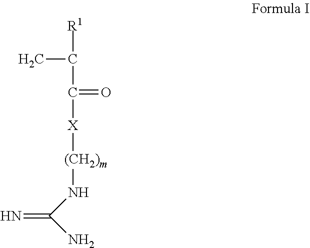
wherein
R¹ is hydrogen or an alkyl group;
X is oxygen or NR⁵; and
R⁵ is hydrogen or an alkyl group, and m is from 1 to 10, or the pharmaceutically-acceptable salt thereof.

136. The method of claim 135, wherein the polycation comprises a copolymer formed by polymerization of the monomer defined by Formula I and methacrylamide.

137. The method of any of claims 135-136, wherein R¹ is methyl, X is NH, and m is 3.

138. The method of any of claims 118-137, wherein the polyanion comprises two or more groups chosen from carboxylate groups, sulfate groups, sulfonate groups, borate groups, boronate groups, phosphonate groups, phosphate groups, or any combination thereof.

139. The method of any of claims 118-138, wherein the polyanion comprises a polyphosphate.

140. The method of claim 139, wherein the polyphosphate comprises a natural polymer or a synthetic polymer.

141. The method of any of claims 139-140, wherein the polyphosphate comprises a polyphosphoserine.

142. The method of any of claims 139-141, wherein the polyphosphate comprises a polyacrylate comprising two or more pendant phosphate groups.

143. The method of any of claims 139-142, wherein the polyphosphate comprises a copolymer derived from a phosphate acrylate and/or phosphate methacrylate and one or more additional polymerizable monomers.

144. The method of any of claims 118-143, wherein the polyanion comprises from 3-10 phosphate groups.

145. The method of any of claims 118-144, wherein the polyanion comprises an inorganic polyphosphate or a phosphorylated sugar.

146. The method of any of claims 118-145, wherein the polyanion comprises inositol hexaphosphate.

147. The method of any of claims 118-146, wherein the polycation, the polyanion, or any combination thereof further comprises at least one crosslinkable group.

148. The method of any of claims 1-115, wherein the fluid biomaterial comprises a composition that reacts in situ in the LAA to form an interpenetrating polymer network (IPN).

149. The method of claim 148, wherein the IPN comprises at least one hydrophilic polymer.

150. The method of any of claims 148-149, wherein the IPN comprises at least one polysiloxane.

151. The method of any of claims 148-150, wherein the composition reacts in situ in the LAA by simultaneous polymerization, crosslinking, or a combination thereof to form the IPN.

152. The method of any of claims 1-115, wherein the fluid biomaterial comprises a crosslinkable biomaterial.

153. The method of claim 152, wherein the crosslinkable biomaterial comprises a first precursor molecule and a second precursor molecule.

154. The method of claim 153, wherein the first precursor molecule comprises an oligomer or polymer having one or more first reactive groups, each first reactive group comprising one or more pᵢ bonds, and the second precursor molecule comprises an oligomer or polymer having one or more second reactive groups, each second reactive group comprising one or more pᵢ bonds, and
wherein the first reactive group is reactive with the second reactive group to form a covalent bond between the first precursor molecule and the second precursor molecule.

155. The method of claim 154, wherein the first reactive group and the second reactive group undergo a cycloaddition reaction.

156. The method of any of claims 153-155, wherein the first precursor molecule comprises an oligomer or polymer having one or more nucleophilic groups, and the second precursor molecule comprises an oligomer or polymer having one or more conjugated unsaturated groups.

157. The method of any of claims 153-156, wherein the first precursor molecule comprises a poly(alkylene oxide)-based oligomer or polymer having x nucleophilic groups, wherein x is an integer greater than or equal to 2.

158. The method of claim 157, wherein x is an integer of from 2 to 8.

159. The method of claim 157 or 158, wherein X is an integer of from 2 to 6.

160. The method of any of claims 157-159, wherein the poly(alkylene oxide)-based oligomer or polymer comprises poly(ethylene glycol).

161. The method of any of claims 157-160, wherein the nucleophilic groups are selected from the group consisting of sulfhydryl groups and amino groups.

162. The method of any of claims 153-161, wherein the first precursor molecule has a molecular weight of from about 1 kDa to about 10 kDa.

163. The method of any of claims 153-162, wherein the first precursor molecule comprises pentaerythritol poly(ethylene glycol)ether tetrasulfhydryl.

164. The method any of claims 153-163, wherein the second precursor molecule comprises a biomacromolecule having y conjugated unsaturated groups, wherein y is an integer greater than or equal to 2.

165. The method of claim 164, wherein y is an integer of from 2 to 100.

166. The method of claim 164 or 165, wherein y is an integer of from 2 to 25.

167. The method of any of claims 164-166, wherein the biomacromolecule comprises a polysaccharide.

168. The method of claim 167, wherein the polysaccharide comprises dextran or a derivative thereof.

169. The method of any of claims 167-168, wherein the conjugated unsaturated groups are selected from the group consisting of vinyl sulfone groups and acryl groups.

170. The method of any of claims 153-169, wherein the second precursor molecule has a molecular weight of from about 2 kDa to about 250 kDa.

171. The method of any of claims 153-170, wherein the second precursor molecule has a molecular weight of from about 5 kDa to about 50 kDa.

172. The method of any of claims 153-171, wherein the second precursor molecule comprises dextran vinyl sulfone.

173. The method of any of claims 153-172, wherein the crosslinkable biomaterial further comprises a base.

174. The method of any of claims 1-173, wherein the cure time is less than about 30 minutes, such as less than about 20 minutes or less than about 15 minutes.

175. The method of any of claims 1-174, wherein the cure time is from about 1 minute to about 20 minutes, such as from about 1 minute to about 15 minutes, from about 3 minutes to about 20 minutes, or from about 3 minutes to about 15 minutes.

176. The method of any of claims 1-175, wherein the biocompatible polymeric matrix has a degradation rate such that less than about 10% or less by weight of the biocompatible polymeric matrix degrades within 90 days of curing.

177. The method of any of claims 1-176, wherein the biocompatible polymeric matrix exhibits an equilibrium swelling ratio of from greater than 0 to about 8, such as from greater than 0 to about 6.

178. The method of any of claims 1-177, wherein the biocompatible polymeric matrix exhibits a volumetric swelling ratio of from greater than 0 to about 15, such as from greater than 0 to about 10, or from about 2 to about 8.

179. The method of any of claims 1-178, wherein the biocompatible polymeric matrix has an elastic modulus of from about 5 kPa to about 20 kPa, such as from about 8 kPa to about 12 kPa.

180. The method of any of claims 1-179, wherein the biocompatible polymeric matrix further comprises a bioactive agent.

181. The method of claim 180, wherein the bioactive agent comprises a silencing agent, such as an apoptotic agent.

182. The method of any of claims 179-181, wherein the bioactive agent comprises a contrast agent.

183. The method of any of claims 1-182, wherein the patient exhibits atrial fibrillation.

184. The method of any of claims 1-183, wherein the LAA is trabeculated.

185. The method of any of claims 1-184, wherein the patient has a CHA₂DS₂-VASc score of 2 or more.

186. The method of any of claims 1-185, wherein the patient is contraindicated for anticoagulation therapy.

187. A method of occluding the left atrial appendage (LAA) of a patient comprising injecting a fluid biomaterial comprising a silencing agent dissolved or dispersed therein into the LAA of the patient;
wherein the fluid biomaterial solidifies in situ in the LAA to form a biocompatible polymeric matrix that fills and occupies the LAA.

188. The method of claim 187, wherein the silencing agent comprises an apoptotic agent.
